(19) United States
(12) Patent Application Publication
(10) Pub. No.: US 2019/0032114 A1
(43) Pub. Date: Jan. 31, 2019

(54) POINT-OF-CARE NUCLEIC ACID AMPLIFICATION AND DETECTION

(71) Applicant: TRIV TECH, LLC, Dallas, TX (US)

(72) Inventor: Krutarth TRIVEDI, Plano, TX (US)

(73) Assignee: Triv Tech, LLC, Dallas, TX (US)

(21) Appl. No.: 16/071,455

(22) PCT Filed: Jan. 19, 2017
(86) PCT No.: PCT/US2017/014175

§ 371 Date: Jul. 19, 2018

Related U.S. Application Data
(60) Provisional application No. 62/290,646, filed on Feb. 3, 2016.
(60) Provisional application No. 62/281,124, filed on Jan. 20, 2016.

(51) Int. Cl. C12Q 1/686; B01L 7/00; B01L 3/00; G01N 21/64

(52) U.S. Cl. CPC C12Q 1/686; B01L 7/52; B01L 3/5085; B01L 3/5027; G01N 21/6454; B01L 2300/1861; B01L 2200/147; G01N 2201/0627; B01L 2300/1816; B01L 2300/1827; G01N 21/6486

(57) ABSTRACT

Devices are disclosed for amplifying and detecting analytes, including oligonucleotide targets. The devices may be used for point of care nucleic acid testing. Methods and assays of using the devices are also disclosed.

### CROSS-REFERENCE TO RELATED APPLICATIONS

This application claims the benefit of priority to U.S. Provisional Application Nos. 62/281,124 filed Jan. 20, 2016 and 62/290,646 filed Feb. 3, 2016, the disclosures of which are hereby expressly incorporated by reference in their entireties.

### FIELD

This application relates to methods, assays, and devices for amplifying and/or detecting analytes. In particular, disclosed herein are methods, assays, and devices for amplifying and/or detecting oligonucleotide targets. Also disclosed are methods, assays, and devices for detecting nanoparticles attached to a surface via captured analyte.

### BACKGROUND

Detection of molecular entities, such as small molecules, oligonucleotides or proteins, is typically accomplished by electrochemical or optical techniques such as redox reaction enabled methods, Enzyme Linked Immunoassay (ELISA), Lateral Flow Assay (LFA) or Polymerase Chain Reaction (PCR). Despite the simple structure and compact form factor of modern electrochemical sensors such as blood glucose measurement strips, sensitivities in the range of mM to M, and poor selectivity limits the detection of many biomarkers. On the other hand, ELISA assays, with sensitivities into the picomolar to nanomolar range, enable detection of most biomarkers. However, these techniques are typically complex and require multiple steps to be performed by highly trained personnel. Furthermore, equipment to perform these assays is typically designed for lab-use, making it impractical to adapt these detection technologies toward point-of-care (POC) applications. In addition, widespread use of nucleic acid tests is limited by the complexity, high cost, and long turnaround time associated with current mainstream nucleic acid testing technology. PCR thermal cycling equipment is typically bulky with large power requirements while the complex procedure requires multiple time consuming steps resulting in prolonged time-to-results. Therefore, nucleic acid tests using PCR are limited to centralized laboratory facilities where tests are performed by highly trained personnel. POC nucleic acid diagnostic testing, where tests may be performed in the field or a low resource setting, would benefit from a fast and miniaturized platform technology for PCR amplification.

LFA technology is well suited to certain POC applications due to its simplicity and low cost. However, because most LFAs lack sufficient sensitivity and specificity, early detection and other advanced diagnostics that require quantification or detection in the picomolar to nanomolar are generally unable to be performed using LFAs.

### SUMMARY

Novel schemes and devices for rapid amplification and/or detection of nucleic acids for use in POC nucleic acid tests are disclosed herein. Novel methods, assays, and devices for the detection of biomarkers or analytes using nanoparticle tags that improves sensitivity, specificity and/or performance, and/or that enable a wider range of applications are also disclosed herein. The methods, assays, and devices disclosed herein may be performed for both liquid-phase and solid-phase nucleic acid amplification and detection using PCR or other diagnostic tools and methods. The following presents a summary in order to provide a basic understanding of one or more aspects of the invention. This summary is not an extensive overview of the invention, and is neither intended to identify key or critical elements of the invention, nor to delineate the scope thereof. Rather, the primary purpose of the summary is to present some concepts of the invention as a prelude to a more detailed description that is presented later. The following presents a simplified summary in order to provide a basic understanding of one or more aspects of the invention.

In some embodiments, a nanoparticle assay system comprises an assay cartridge comprising a test region. The test region configured to analyze a sample potentially containing (for example, believed to contain or suspected to contain) an analyte. The test region comprises a capture region. The capture region optionally comprises analyte binding capture probe molecule. If the analyte is present, the analyte binds to the capture region (for example, to the analyte binding capture probe molecule if present). If the analyte is present, a nanoparticle conjugated with analyte binding capture probe molecules binds to the analyte, or optionally another molecule bound to the analyte, that is bound to the capture region. The analyte binding capture probe molecules of the nanoparticle optionally bind to other analytes in the sample, and may optionally be capped by analyte binding capture probe molecules binding to the other analytes. The capture region may optionally have a non-planar surface, for example, due to patterning. A source of radiation can excite the nanoparticles to produce a measurable response. A detector can detect the response of the nanoparticles to radiation from the source. The capture region may be free of or substantially free of unbound nanoparticles and/or sample fluid. The cartridge optionally include various layers and/or devices under the capture region, for example, a reflective layer, a partially reflective layer (for example, reflective to radiation from the source but transmissive to radiation from the nanoparticles), a temperature sensitive device, an infrared sensitive device, a bolometer, and/or the like as well as various optional architecture therefor (for example, thermally insulating layers, electrically insulating layers, reflective layers, isolation structures such as vias and wells, support structures and layers, etc.).

In some embodiments, a nanoparticle assay system comprises a sample potentially containing (for example, believed to contain or suspected to contain) one or more analytes of interest, an assay cartridge comprising a test region including a nonporous and/or non-membranous surface, a test region containing one or more capture regions, analyte binding capture probe molecules on the surface of the capture region(s) and within the test region, nanoparticles conjugated with analyte binding capture probe molecules, a source of radiation wherein radiation excites the nanoparticles to produce a measurable response, a ultraviolet, visible and/or thermal radiation detector, and any subcombinations of the above features.

The test region may comprise a nonporous and/or non-membranous surface comprising polymer, epoxy, plastic, semiconductor, oxide, metal and/or any combination thereof. The surface of the test region may be coated with a reflective material (for example, silver, aluminum) and/or a dielectric mirror stack designed to specifically reflect the incident energy, which may be coated by a thin layer of dielectric. The surface of the test region may comprise three dimensional patterned structures comprising polymer, epoxy, plastic, semiconductor, oxide, metal and/or any combination thereof. The analyte binding capture probe molecules may be coupled to the surface of the test region by linker molecules. The linker molecules may comprise one or more chemical molecules and/or functional silane wherein one end terminal end of the molecule, or chain of molecules, is bound to the surface of the test region and wherein another terminal end of the molecule, or chain of molecules, comprises a functional group capable of binding capture probe molecules. The assay cartridge may comprise multiple capture regions within the test region. The multiple capture regions may be coated with the same or different capture probes. The nanoparticles may comprise one or more layers of gold, silver, carbon, platinum, polymer, plastic, oxide, iron and/or any combination thereof. The geometry of the nanoparticles may comprise spheres, cylinders, rods, core-shell particles, urchins, stars, plates, cubes, porphysomes and/or any combination thereof. Analyte binding capture probe molecules may be coupled to the surface of nanoparticles by linker molecules. The linker molecules may comprise one or more chemical molecules and/or functional silane wherein one terminal end of the molecule, or chain of molecules, is bound to the surface of the nanoparticle and another terminal end of the molecule, or chain of molecules, comprise a functional group capable of binding analyte binding capture probe molecules. The capture probe molecules may comprise chemical molecules, antibody, enzyme, protein, oligonucleotide, single-stranded DNA, double-stranded DNA, aptamer, DNAzyme, aptazyme, a synthetic molecule capable of binding target analyte in sample and/or any combination thereof. The analyte or analytes of interest may comprise oligonucleotides, proteins, antibodies, chemical molecules and/or any combination thereof. The source of radiation may comprise a diode laser, DPSS laser, fiber-coupled laser, light-emitting diode and/or any combination thereof. The radiation detector may comprise a CMOS or CCD device, a photodiode, an infrared camera module, an infrared-sensitive semiconductor chip or circuit, and/or any combination thereof.

In some embodiments, a method of performing a nanoparticle assay using a nanoparticle assay system described herein and/or another nanoparticle assay system comprises dispensing buffer and/or similar solution to the test region, exposing the test region, before contact with any sample solution, to a source of radiation, measuring a baseline reading with the thermal radiation detector, dispensing the sample, containing analyte/analytes of interest to the test region, wherein the sample is propelled towards the test region by external pressure and/or is manipulated by pipetting, allowing the sample to react with the test region for a duration configured so that present analyte(s) of interest to bind to the test region, washing and/or rinsing the test region with buffer and/or similar solution one or multiple times, dispensing a solution containing nanoparticles conjugated with analyte binding capture probe molecules to the test region and allowing reaction for a pre-set period of time, washing and/or rinsing the test region with buffer and/or similar solution one time or multiple times, exposing the test region to said radiation for a pre-set period of time, detecting the infrared radiation emitted from the test region with the thermal radiation detector, calculating and reporting the concentration of analyte by analyzing the detected thermal radiation signal, and any subcombinations of the above features.

Measuring the baseline reading may be done without exposing the test region to a source of radiation. The test region may be not washed before exposing the test region to a solution containing nanoparticles conjugated with analyte binding capture probe molecules. The test region may be not washed before exposing the test region to radiation and detecting the thermal radiation signal. The method may further comprise removing the bulk of the fluid over the test region prior to exposing the test region to incident energy and measuring the thermal response.

In some embodiments, a method of performing a nanoparticle assay using a nanoparticle assay system described herein and/or another nanoparticle assay system comprises dispensing buffer and/or similar solution to the test region, exposing the test region, before contact with any sample solution, to a source of radiation, measuring a baseline reading with the thermal radiation detector, mixing the sample, containing analyte/analytes of interest, with solution containing nanoparticles conjugated with analyte binding capture probe molecules for a pre-set period of time, dispensing the solution containing nanoparticle/analyte complexes to the test region, wherein the sample is propelled towards the test region by external pressure and/or is manipulated by pipetting, allowing the solution to react with the test region for a duration configured so that nanoparticle/analyte complexes to bind to the surface of the capture region(s), washing and/or rinsing the test region with buffer or similar solution one or multiple times, exposing the test region to said radiation for a pre-set period of time, detecting the infrared radiation emitted from the test region with the thermal radiation detector, calculating and reporting the concentration of analyte by analyzing the detected thermal radiation signal; and any subcombinations of the above features.

Measuring the baseline reading may be without exposing the test region to a source of radiation. The test region may be not washed before exposing the test region to radiation and detecting the thermal radiation signal. The method may further comprise removing the bulk of the fluid over the test region prior to exposing the test region to incident energy and measuring the thermal response.

In some embodiments, a method of performing a nanoparticle assay using a nanoparticle assay system described herein and/or another nanoparticle assay system comprises dispensing buffer and/or similar solution to the test region, exposing the test region, before contact with any sample solution, to a source of radiation, measuring a baseline reading with the thermal radiation detector, mixing the sample with capture probe molecules, where the capture probe molecules may be a single type for capture of single target analyte or different types for capture of multiple analytes, dispensing the solution with capture probe molecules bound to target analyte(s) towards the test region, allowing the solution to react with the test region for a pre-set period of time, causing the capture probe/analyte complexes to bind to capture probe molecules on the surface of the capture region, washing and/or rinsing the test region with buffer and/or similar solution one or multiple times, providing nanoparticles modified with capture probe molecules which bind to all of the capture probes attached to the analytes at the surface of the test region, even different capture probes attached to different analytes, dispensing the solution with the nanoparticle/probe complexes toward the test region, allowing the solution to react with the capture probes exposed on the surface of the test region for duration configured so that the nanoparticles bind to the capture probes on the surface, washing and/or rinsing the test region with buffer and/or similar solution one time or multiple times, exposing the test region to incident radiation for a pre-set period of time, detecting the infrared radiation emitted from the test region with the thermal radiation detector, calculating and reporting the concentration of analyte by analyzing the detected thermal radiation signal, and any subcombinations of the above features.

Measuring the baseline reading may be without exposing the test region to a source of radiation. The test region may be not washed before dispensing solution with the nanoparticle/probe complexes toward the test region. The test region may be not washed before exposing the test region to radiation and detecting the thermal radiation signal. The method may further comprise removing the bulk of the fluid over the test region prior to exposing the test region to incident energy and measuring the thermal response.

In some embodiments, a method of performing a nanoparticle assay using a nanoparticle assay system described herein and/or another nanoparticle assay system comprises dispensing buffer and/or similar solution to the test region, exposing the test region, before contact with any sample solution, to a source of radiation, measuring a baseline reading with the thermal radiation detector, denaturing the double stranded amplified DNA, wherein the DNA is a product of an amplification process, to separate the DNA into two sets of single stranded DNA, denoted A1 and A2, allowing one set of strands, with sequence A1, to bind to a surface conjugated with single stranded DNA partially or fully complementary to sequence A1, providing a test region with a surface conjugated to single stranded DNA capture probes, with sequence partially complementary to A2, dispensing the remaining separated single stranded DNA, with sequence A2, to the test region and allowing it to hybridize to the capture probes on the surface of the test region, washing and/or rinsing the test region with buffer and/or similar solution one time or multiple times, providing nanoparticles conjugated to single stranded DNA capture probes, with sequence partially complementary to A2, dispensing the solution with the nanoparticle/capture probe complexes to the test region and allowing hybridization to the partially hybridized strands, with sequence A2, exposed on the surface of the test region, washing and/or rinsing the test region with buffer and/or similar solution one time or multiple times, exposing the test region to incident radiation for a pre-set period of time, detecting the infrared radiation emitted from the test region with the thermal radiation detector, calculating and reporting the concentration and/or the presence of any hybridized DNA by analyzing the detected thermal radiation signal, and any subcombinations of the above features.

Measuring the baseline reading may be without exposing the test region to a source of radiation. The test region may be not washed before dispensing solution with the nanoparticle/probe complexes toward the test region. The test region may be not washed before exposing the test region to radiation and detecting the thermal radiation signal. The method may further comprise removing the bulk of the fluid over the test region prior to exposing the test region to incident energy and measuring the thermal response.

In some embodiments, a method of performing a nanoparticle assay using a nanoparticle assay system described herein and/or another nanoparticle assay system comprises dispensing buffer and/or similar solution to the test region, exposing the test region, before contact with any sample solution, to a source of radiation, measuring a baseline reading with the thermal radiation detector, providing nanoparticles conjugated to single stranded DNA, with sequence B1, providing a test region with a surface conjugated to single stranded DNA capture probes, with sequence partially or fully complementary to sequence B1, dispensing the solution with the nanoparticle/DNA complexes to the test region and allowing hybridization to single stranded DNA exposed on the surface of the test region, washing and/or rinsing the test region with buffer and/or similar solution one time or multiple times, exposing the test region to incident radiation for a pre-set period of time, detecting the infrared radiation emitted from the test region with the thermal radiation detector, calculating and reporting the concentration and/or the presence of any hybridized DNA by analyzing the detected thermal radiation signal; and any subcombinations of the above features.

Measuring the baseline reading may be without exposing the test region to a source of radiation. The test region may be not washed before exposing the test region to radiation and detecting the thermal radiation signal. The bulk of the fluid over the test region may be removed prior to exposing the test region to incident energy and measuring the thermal response.

In some embodiments, a method of measuring a signal as described in the methods herein or other methods may comprise exposing the test region to incident radiation, periodically toggled on and off at a frequency, detecting infrared radiation emitted from the test region with a thermal detector, recording and measuring a derivative of the detected infrared radiation to determine a rate of change of emitted thermal radiation with respect to the incident radiation's toggle frequency, correlating the rate of change of emitted thermal radiation to concentration of bound nanoparticles and analyte, and any subcombinations of the above features.

In some embodiments, a method of calculating and reporting concentration of an analyte comprises subtracting a detected thermal radiation signal from a baseline reading to create a result, comparing the result with pre-determined calibrated values stored in memory, correlating an amplitude, maximum, and/or average value of the detected thermal radiation signal to concentration of bound nanoparticles and analyte, and any subcombinations of the above features.

In some embodiments, a method of calculating and reporting concentration of an analyte comprises subtracting a detected thermal radiation signal from a baseline reading to create a result, comparing the result with a similar measurement performed on a calibration region or regions, wherein the calibration region/regions have a known quantity of nanoparticles bound to the surface, correlating an amplitude, maximum, and/or average value of the detected thermal radiation signal to concentration of bound nanoparticles and analyte, and any subcombinations of the above features.

The test region may comprise a material which is transparent to radiation and does not absorb incident radiation or absorbs a known quantity of incident radiation. The test region may comprise a thin sheet of infrared transparent plastic, semiconductor, metal oxide, chalcogenide, semiconductor oxide and/or any combination thereof. The surface of the infrared transparent material may comprise one or more layers of three dimensional patterned structures made from polymer, epoxy, plastic, semiconductor, oxide, metal and/or any combination thereof.

In some embodiments, a method of fabricating a test region of an assay cartridge comprises providing a supporting substrate composed of one or more layers, forming an opening or hole in the supporting substrate at the location of the test region, with the hole cutting through the entire thickness of the supporting substrate, adhering a layer of material of the test region of a nanoparticle assay system as described herein or another nanoparticle assay system to a surface of the supporting substrate via an adhesive liner including a matching opening or hole, depositing a thin layer of infrared transparent material for attachment of conjugation chemistry, cleaning and surface treatment by plasma and/or chemical means to activate the surface for chemical/bio conjugation, attaching conjugation chemistry to a surface of the test region, selectively placing capture probes only to the surface of the test region which lies directly over the hole or opening in the supporting substrate, and any subcombinations of the above features.

In some embodiments, a method of measuring a radiation signal of a nanoparticle assay using the test region of the test region of a nanoparticle assay system as described herein or another nanoparticle assay system comprises placing a detector behind the assay cartridge such that a camera images the test region from the backside, or the side opposite to the surface where the assay takes place, measuring radiation emitted through the surface of the test region at the location of the hole or opening in the test region, and any subcombinations of the above features.

In some embodiments, a method of performing a nanoparticle assay using a nanoparticle assay system described herein and/or another nanoparticle assay system comprises providing an assay cartridge having one or more test regions as described herein and/or another assay cartridge, flowing buffer and/or similar solution to the test region, exposing the test region, before contact with any sample solution, to a source of radiation and measuring a baseline reading, of light emitted through the transparent surface of the test region, with the radiation detector using a method described herein and/or another method, dispensing the sample, containing analyte/analytes of interest to the test region, wherein the sample is propelled towards the test region by external pressure and/or is manipulated by pipetting, allowing the sample to react with the test region for a duration configured so that present analyte(s) of interest to bind to the test region, washing and/or rinsing the test region with buffer and/or similar solution one or multiple times, exposing the test region to a solution containing nanoparticles conjugated with analyte binding capture probe molecules for a pre-set period of time, washing and/or rinsing the test region with buffer and/or similar solution one time or multiple times, exposing the test region to incident radiation, detecting the scattered radiation emitted through the transparent surface of the test region with the radiation detector using a method described herein and/or another method, calculating and reporting the concentration of analyte by analyzing the detected thermal radiation signal, and any subcombinations of the above features.

The test region may be not washed before exposing the test region to radiation and detecting the radiation signal. The test region may be not washed before dispensing solution with the nanoparticle complexes toward the test region. The test region may be not washed before exposing the test region to radiation and detecting the thermal radiation signal. The method may further comprise removing the bulk of the fluid over the test region prior to exposing the test region to incident energy and measuring the thermal response.

In some embodiments, a method of performing a nanoparticle assay using a nanoparticle assay system described herein and/or another nanoparticle assay system, where the method comprises a method described herein and/or another method, comprises prior to exposing a test region to incident energy and measuring a signal providing a second set of nanoparticles modified with capture probe molecules to attach to the analyte or the capture probe molecules on the first set of nanoparticles already attached to the surface of the capture region, dispensing a solution containing the second set of modified nanoparticles towards the test region, allowing the solution to react with the first set of nanoparticles already attached the surface of the test region for duration configured so that the second set of nanoparticles bind to the first set of nanoparticles already attached to the surface, washing and/or rinsing the test region with buffer and/or similar solution one or multiple times, and any subcombinations of the above features.

In some embodiments, a nanoparticle assay system comprises a sample potentially containing one or more analytes of interest, an assay cartridge comprising a detection region including at least one electronic sensor chip, an electronic sensor chip comprising a test region containing one or more capture regions and including at least one sensing device, analyte binding capture probe molecules on the surface of the capture region(s) on the sensing device, nanoparticles conjugated with analyte binding capture probe molecules, a source of radiation wherein the radiation excites the nanoparticles to produce a measurable response, and any subcombinations of the above features.

The detection region may comprise an array of electronic sensor chips. The test region may comprise an array of sensing devices. Individual sensing devices in the array may be isolated from each other at a substrate level by trenches between the individual sensing devices. Each of the sensing devices in the array may be functionalized with same capture probe molecules. Each of the sensing devices in the array may be functionalized with different capture probe molecules. Groups of sensing devices in the array may be functionalized with capture probe molecules being different between the groups. The sensing device may comprise one or more semiconductor devices, diodes, transistors, resistors, thermistors, resistance thermometer devices, thermocouples, thermopiles, thermostats, bolometers, microbolometers or any combination thereof. The analyte binding capture probe molecules may be coupled to the surface of the at least one sensing device by linker molecules. The linker molecules may comprise one or more chemical molecules or functional silane. One end terminal end of the linker molecule, or chain of molecules, may be bound to the surface of the sensing device. The other end of the linker molecule, or chain of molecules, may comprise a functional group capable of binding capture probe molecules. The nanoparticles may comprise one or more layers of gold, silver, carbon, platinum, polymer, plastic, oxide, iron or any combination thereof. The geometry of the nanoparticles may comprise spheres, cylinders, rods, core-shell particles, urchins, stars, plates, cubes, porphysomes or any combination thereof. Analyte binding capture probe molecules may be coupled to the surface of nanoparticles by linker molecules. The linker molecules may comprise one or more chemical molecules or functional silane. One terminal end of the linker molecule, chain of molecules, may be bound to the surface of the nanoparticle. The other end of the linker molecule, or chain of molecules, may comprise a functional group capable of binding analyte binding capture probe molecules. The capture probe molecules may comprise chemical molecules, antibody, enzyme, protein, oligonucleotide, single-stranded DNA, double-stranded DNA, aptamer, DNAzyme, aptazyme, a synthetic molecule capable of binding the analyte or analytes of interest or any combinations thereof. The analyte or analytes of interest may comprise oligonucleotides, proteins, antibodies, chemical molecules or any combinations thereof. The source of radiation may comprise a diode laser, DPSS laser, fiber-coupled laser, light-emitting diode or any combination thereof.

In some embodiments, a sensor device for detecting nanoparticles in a nanoparticle assay system described herein and/or another nanoparticle assay system comprises an active element which is sensitive to changes in temperature, a layer of thermally insulating material over the active element, the layer of thermally insulating material including one or more patterned openings, a layer of reflective material over the active element, the layer of reflective material including one or more patterned openings, a layer of capping material over the active element, the layer of thermally insulating material, and the layer of reflective material, the layer of capping material comprising the surface of the capture region, a material comprising a thermal mass for heat transfer from the capture region to the active element, and any subcombination of the above features.

The openings in the layers over the active element may be aligned such that a continuous opening through the layers exposes the active element or a layer over the active element. A thermally conducting material may fill the openings. The capping layer may include openings aligned with the openings in the layers beneath the capping layer. A thermally conducting material may fill the openings in the capping layer. The capping layer may cover the thermal mass. The thermal mass may comprise an oxide, a metal, carbon nanotubes, graphene, graphite or any combination thereof.

In some embodiments, a sensor device for detecting nanoparticles in a nanoparticle assay system described herein and/or another nanoparticle assay system comprises an active element which is sensitive to infrared radiation, a layer of thermally insulating material over the active element, a layer of reflective material over the active element, a layer of capping over the active element, the layer of thermally insulating material, and the layer of reflective material, the layer of capping material comprising the surface of the capture region, and any subcombination of the above features.

The active element may comprise one or more semiconductor devices, diodes, transistors, resistors, thermistors, resistance thermometer devices, thermocouples, thermopiles, thermostats, bolometers, microbolometers or any combination thereof. The thermally insulating material may comprise an oxide, polymer, parylene, aerogel, an air gap or any combination thereof. The reflective layer may comprise a metal, an oxide, a stack of oxides, a dielectric mirror or any combination thereof. The capping material may comprise an oxide, polymer, parylene or any combination thereof.

In some embodiments, a method of fabricating a bolometer or microbolometer device, the method comprises forming a reflective layer on a substrate, forming a thermally insulating layer over the substrate, forming a thermistor layer over the thermally insulating layer, where forming the thermistor layer comprises forming at least two electrical contacts, forming openings in the thermally insulating layer, forming electrically conductive vias in the openings, electrically connecting the vias to the electrical contacts of the thermistor layer, forming a layer of thermally insulating material over the thermistor layer, forming one or more materials comprising a reflective layer over the thermistor layer, forming at least one via through the thermally insulating and reflective layers, filling the via with an infrared reflecting or absorbing material, forming a layer of capping material over the layer of thermally insulating material, the one or more materials comprising a reflective layer, and the vias in the thermally insulating and reflective layers, the capping layer comprising the surface of the capture region, and any subcombination of the above features.

Accordingly, some aspects of the present invention relate to the following embodiments:

1. A cartridge for performing one or more nucleic acid amplification reactions comprising: one or more reaction zones configured to receive reagents for performing said one or more nucleic acid amplification reactions involving a heating process; and a heat generation layer in thermal communication with said one or more reaction zones, wherein said heat generation layer is configured to generate heat for at least one heating cycle via light provided by a light source.

2. The cartridge of embodiment 1, wherein the heat generation layer comprises pigments, dyes, pigmented or dyed plastic film or sheet, semiconductors, compound semiconductors, carbon nanotubes, fullerenes, graphene, oxides, graphene-oxide, metal-oxide, semiconductor-oxide, polymer, plastic, metal, metal-alloy, germanium, polyimide, glass, nanoparticles and/or microparticles, or a combination thereof.

3. The cartridge of any one of embodiments 1-2, wherein the heat generation layer comprises particles or beads.

4. The cartridge of any one of embodiments 1-3, wherein the one or more reaction zones are configured as one or more array of wells, holes, grooves, channels, or trench structures.

5. The cartridge of any one of embodiments 1-4, wherein the cartridge comprises a substrate comprising a material selected from the group consisting of semiconductor, metal, FR-4, polymer, plastic, epoxy, resin, glass, silicone, rubber, a track-etched membrane, and a combination thereof.

6. The cartridge of embodiment 5, wherein the substrate is transparent to light having a wavelength of about 400 nanometers to about 1 micrometer or any range therebetween.

7. The cartridge of embodiment 5, wherein the substrate is transparent to light having a wavelength of about 5 micrometers to about 13 micrometers or any range therebetween.

8. The cartridge of any one of embodiments 5-7, wherein the heat generation layer and the substrate combined have an emissivity of about 0.1 to about 1 in mid to far infrared range, or any range therebetween.

9. The cartridge of any one of embodiments 5-8, wherein the heat generation layer and the substrate combined have an emissivity of about 0.5 to about 1 in mid to far infrared range, or any range therebetween.

10. The cartridge of any one of embodiments 5-9, wherein the heat generation layer and the substrate combined have an emissivity of about 0.8 to about 1 in mid to far infrared range, or any range therebetween.

11. The cartridge of any one of embodiments 1-10, further comprising a thermal conduction layer in thermal communication with said heat generation layer.

12. A reader configured to receive the cartridge of any one of embodiments 1-11, said reader comprising: a light source configured to provide light to said heat generation layer to generate heat for said heating process; a detector configured to detect amplification products produced by said one or more nucleic acid amplification reactions; and a thermal sensor which detects infrared light emitted from said heat generation layer or a circuit which communicates a signal indicative of temperature, said signal generated by a contact based temperature sensor in thermal communication with the heat generation layer in said cartridge.

13. The reader of embodiment 12, wherein the reader is configured to perform one or more heating cycles.

14. The reader of any one of embodiments 12-13, wherein the light source comprises a light emitting diode, an array of light emitting diodes, a laser diode, an array of laser diodes, a DPSS laser, an array of DPSS lasers, at least one focusing lens, at least one collimating lens, or a combination thereof.

15. The reader of any one of embodiments 12-14, wherein the detector is configured to detect fluorescence emitted by said amplification products.

16. The reader of any one of embodiments 12-15, wherein the thermal sensor comprises an infrared sensor.

17. The reader of embodiment 16, wherein the infrared sensor is a charge coupled device (CCD), complementary metal-oxide semiconductor device (CMOS), photovoltaic device, photodiode device, photoconductor device, thermopile device, bolometer device or a combination thereof.

18. The reader of any one of embodiments 12-17, wherein the infrared light is mid- to far-infrared.

19. The reader of any one of embodiments 12-18, wherein the infrared light has a wavelength that is from about 4 to about 16 micrometers, or any range there between.

20. The reader of any one of embodiments 12-19, wherein the infrared light has a wavelength that is from about 8 to about 14 micrometers, or any range there between.

21. The reader of any one of embodiments 12-15, wherein the thermal sensor comprises a contact temperature sensor.

22. The reader of embodiment 21, wherein the contact temperature sensor is a thermocouple, a resistance temperature detector, a thermistor, or a combination thereof.

23. The reader of any one of embodiments 21-22, wherein the contact temperature sensor is not in contact with a liquid in a sample in which an amplification reaction is being performed.

24. The reader of any one of embodiments 12-23, further comprising a cooling system configured to cool the one or more nucleic acid amplification reactions.

25. The reader of any one of embodiments 12-24, further comprising a detector configured to detect amplification products generated by said one or more nucleic acid amplification reactions.

26. The reader of embodiment 25 wherein said detector is configured to detect fluorescence emitted by said amplification products.

27. The reader of any one of embodiments 12-26, wherein said reader is a point of care reader.

28. A system comprising a cartridge of any one of embodiments 1-11 and a reader of any one of embodiments 12-27.

29. A method of performing one or more nucleic acid amplification reactions comprising at least one heating cycle, said method comprising receiving one or more samples at one or more reaction zones, generating heat at said one or more reaction zones by illuminating a heat generation layer in thermal communication with said one or more reaction zones, and performing nucleic acid amplification reactions on said one or more samples.

30. The method of embodiment 29, further comprising detecting a temperature of the heat generation layer.

31. The method of any one of embodiments 29-30, further comprising detecting amplification products.

32. The method of any one of embodiments 29-31, wherein the heat generation layer is illuminated by a light source comprising a light emitting diode, an array of light emitting diodes, a laser diode, an array of laser diodes, a DPSS laser, an array of DPSS lasers, at least one focusing lens, at least one collimating lens, or a combination thereof.

33. The method of embodiment 30, wherein detecting the temperature of the heat generation layer comprises detecting infrared light emitted from the heat generation layer using an infrared sensor comprising a charge coupled device (CCD), complementary metal-oxide semiconductor device (CMOS), photovoltaic device, photodiode device, photoconductor device, thermopile device, bolometer device or a combination thereof.

34. The method of any one of embodiments 30 or 33, wherein detecting the temperature of the heat generation layer comprises detecting the temperature using a contact temperature sensor comprising a thermocouple, a resistance temperature detector, a thermistor, or a combination thereof.

35. The method of any one of embodiments 29-34, wherein method comprises one or more heating cycles.

36. The method of any one of embodiments 29-35, wherein the method comprises one or more cooling cycles.

37. A point-of-care system for amplification and detection of nucleic acid molecules, comprising: a test cartridge configured to perform nucleic acid amplification; a reader device configured to detect nucleic acid amplification products; and an energy source configured to heat a liquid sample in which a nucleic acid amplification process is performed.

38. The system of embodiment 37, wherein said energy source comprises a light source.

39. The system of any one of embodiments 37-38, wherein the test cartridge is configured to receive the liquid sample.

40. The system of any one of embodiments 37-39, wherein the test cartridge comprises one or more reaction zones, a substrate, or a heat generation layer.

41. The system of embodiment 40, wherein the one or more reaction zones is configured as one or more array of wells, holes, grooves, channels, or trench structures.

42. The system of embodiment 40, wherein the substrate is configured as a base for coatings, depositions, and/or fabrications of one or more 3D pattern layers, heat generation layers, thermal conduction layers, passivation layers, sample confinement layers, capping or encapsulation layers, or a combination thereof.

43. The system of any one of embodiments 40-42, wherein the substrate comprises a material selected from the group consisting of semiconductor, metal, FR-4, polymer, plastic, epoxy, resin, glass, silicone, rubber, a track-etched membrane, and a combination thereof.

44. The system of embodiment 43, wherein the substrate material is at least partially transparent in the wavelength range between 400 nanometers to 1 micrometer.

45. The system of embodiment 43, wherein the substrate material is at least partially transparent in the mid to long infrared spectrum wavelength in a range between 5 micrometers to 13 micrometers.

46. The system of embodiment 42, wherein the one or more 3D pattern layers are positioned under the heat generation layer and are configured to increase the surface area of the heat generation layer and/or increase the height of the heat generation layer so as to reduce the length that the reactants must diffuse to reach the heat generation layer.

47. The system of any one of embodiments 42 or 46, wherein the one or more 3D pattern layers comprises a material selected from the group consisting of polymer, plastic, silicone, rubber, glass, metal-oxide, semiconductor-oxide, and a combination thereof.

48. The system of any one of embodiments 42 or 46-47, wherein the one or more 3D pattern layers is a planar layer.

49. The system of any one of embodiments 42 or 46-48, wherein the one or more 3D pattern layers comprise patterned and/or deposited features and/or structures.

50. The system of embodiment 49, wherein the features and/or structures comprise one or more arrays of pillars, lines, line and space gratings, pyramids, triangles, trenches, spheres, or a combination thereof.

51. The system of any one of embodiments 49-50, wherein the features and/or structures are deposited and/or fabricated by photolithography, fused deposition modeling 3D printing, stereolithography 3D printing, selective laser sintering 3D printing, inkjet printing, molding, microarray printing/blotting/spotting, or a combination thereof.

52. The system of embodiment 47, wherein the material in the one or more 3D pattern layers is at least partially transparent in the wavelength range between 400 nanometers to 1 micrometer.

53. The system of embodiment 47, wherein the material in the one or more 3D pattern layers is at least partially transparent in the mid to long infrared spectrum wavelength in a range between 5 micrometers to 13 micrometers.

54. The system of embodiment 40, wherein the heat generation layer is positioned on top of a 3D pattern layer.

55. The system of any one of embodiments 40 or 54, wherein the heat generation layer is a light absorbing layer.

56. The system of embodiment 55, wherein the light absorbing layer is configured to absorb light energy input from the energy source and transform it into thermal energy.

57. The system of embodiment 56, wherein the thermal energy produced in the light absorbing layer is proportional to an amount of energy output from the energy source.

58. The system of any one of embodiments 55-57, wherein the light absorbing layer comprises a material selected from the group consisting of pigment, dye, semiconductor, compound semiconductor, carbon nanotubes, fullerenes, graphene, graphene-oxide, metal-oxide, semiconductor-oxide, polymer, plastic, metal, metal-alloy, and a combination thereof.

59. The system of any one of embodiments 55-58, wherein the light absorbing layer comprises germanium, polyimide, pigment or dye or a pigmented or dyed plastic film or sheet, nanoparticles and/or microparticles composed of metal, semiconductor, compound semiconductor, polymer, plastic, oxide, glass, or a combination thereof.

60. The system of embodiment 59, wherein the nanoparticles and/or microparticles are infused with and/or capped with light absorbing materials of embodiment 58.

61. The system of any one of embodiments 40 or 54-60, wherein the heat generation layer is a resistive heater layer.

62. The system of embodiment 61, wherein the resistive heater layer is configured as one or more traces and/or circuits to dissipate or absorb the voltage and/or current energy input from the energy source and transform it into thermal energy.

63. The system of embodiment 62, wherein thermal energy is produced in the one or more trace and/or circuit(s) of the resistive heater layer, which is proportional to the resistance of the trace(s) and/or circuit(s) of the resistive heater layer and the current flowing from the energy source and into the trace(s) and/or circuit(s) of the resistive heater layer.

64. The system of embodiment 63, wherein the trace(s) and/or circuit(s) of the resistive heater layer comprises semiconductor, compound semiconductor, carbon nanotubes, fullerenes, graphene, graphene-oxide, metal-oxide, semiconductor-oxide, metal, metal-alloy or any combination thereof.

65. The system of any one of embodiments 37-64, wherein the reader device is configured to receive the test cartridge.

66. The system of any one of embodiments 37-65, wherein the energy source comprises a light emitting diode, an array of light emitting diodes, a laser diode, an array of laser diodes, a DPSS laser, an array of DPSS lasers, at least one focusing lens, at least one collimating lens, or a combination thereof.

67. The system of any one of embodiments 37-66, further comprising one or more thermal sensors.

68. The system of embodiment 67, wherein the one or more thermal sensors comprise one or more non-contact infrared detectors.

69. The system of embodiment 68, wherein the infrared detector is a charge coupled device (CCD), complementary metal-oxide semiconductor device (CMOS), photovoltaic device, photodiode device, photoconductor device, thermopile device, bolometer device or any combination thereof.

70. The system of any one of embodiments 66-69, wherein the one or more thermal sensors are positioned to be under or above the liquid sample.

71. The system of any one of embodiments 66-70, wherein the one or more thermal sensors comprise one or more contact temperature sensors.

72. The system of embodiment 71, wherein the contact temperature sensor is a thermocouple, a resistance temperature detector, a thermistor, or a combination thereof.

73. The system of any one of embodiments 66-72, wherein the one or more thermal sensors are positioned to be in contact with the liquid sample.

74. The system of any one of embodiments 66-73, wherein the one or more thermal sensors are placed inside a sample confinement layer and in contact with the liquid sample.

75. The system of any one of embodiments 66-74, wherein the one or more thermal sensors are configured as a resistance temperature device or thermistor patterned and/or fabricated in close proximity to a heat generation layer.

76. The system of any one of embodiments 37-75, further comprising a thermal conduction layer.

77. The system of embodiment 76, wherein the thermal conduction layer is configured to facilitate heat transfer to the liquid sample and/or heat transfer from the liquid sample.

78. The system of any one of embodiments 76-77, wherein the thermal conduction layer comprises a material selected from the group consisting of metal, metal-alloy, semiconductor, compound semiconductor, graphene, carbon nanotubes, fullerenes, nanoparticles, microparticles, metal-oxide, semiconductor-oxide, and a combination thereof.

79. The system of any one of embodiments 37-78, further comprising a passivation layer.

80. The system of embodiment 79, wherein the passivation layer is configured to form an interface between the liquid sample and the energy source.

81. The system of any one of embodiments 79-80, wherein the passivation layer comprises a material selected from the group consisting of metal-oxide, semiconductor-oxide, glass, photoresist, plastic, polymer, semiconductor, metal, metal-alloy, and a combination thereof.

82. The system of any one of embodiments 79-81, wherein the passivation layer comprises a surface, wherein the surface of the passivation layer is coated or modified with chemical molecules, silane, protein, nucleic acids, or a combination thereof.

83. The system of any one of embodiments 37-82, further comprising a liquid sample comprising DNA, polymerase, DNase inhibitor, forward primer sequence strands, reverse primer sequence strands, free unlabeled nucleotides, free nucleotides labeled with one or more molecules, water, buffer salts, metal ions, or a combination thereof.

84. The system of any one of embodiments 37-82, further comprising a liquid sample comprising RNA or mRNA, reverse transcriptase, polymerase, RNase inhibitor, forward primer sequence strands, reverse primer sequence strands, free unlabeled nucleotides, free nucleotides labeled with one or more molecules, water, buffer salts, metal ions, or a combination thereof.

85. The system of any one of embodiments 37-84, further comprising a sample confinement layer, wherein the sample confinement layer comprises a well, hole, groove, or trench structure.

86. The system of embodiment 85, wherein the well, hole, groove or trench structure is fabricated from metal-oxide, semiconductor-oxide, metal, metal-alloy, glass, plastic, polymer, photoresist, silicone, rubber, or a combination thereof.

87. The system of any one of embodiments 85-86, wherein the sample confinement layer is coated with thermally conductive material selected from the group consisting of metal, metal-alloy, semiconductor, compound semiconductor, graphene, fullerenes, carbon nanotubes, nanoparticles, microparticles, and a combination thereof.

88. The system of embodiment 87, wherein the thermally conductive material is coated with a passivating material selected from the group consisting of metal-oxide, semiconductor-oxide, glass, photoresist, plastic, polymer, semiconductor, metal, metal-alloy, and a combination thereof.

89. The system of any one of embodiments 37-88, further comprising a capping or encapsulation layer, wherein the capping or encapsulation layer is configured to prevent evaporation of the liquid sample.

90. The system of embodiment 89, wherein the capping or encapsulation layer comprises a film of oil, plastic, or glass.

91. The system of any one of embodiments 37-90, further comprising a supplementary heating device, wherein the supplementary heating device is a thermoelectric device, a heat block, a resistive heater, a printed circuit board heater, a flexible circuit heater, or a combination thereof.

92. The system of any one of embodiments 37-91, further comprising a supplementary cooling device, wherein the supplementary cooling device is a heatsink, a fan, a thermoelectric device, a Peltier cooler, or a combination thereof.

93. The system of any one of embodiments 37-92, wherein said system is configured to conduct a PCR reaction at or in close proximity of the surface of the reaction zone.

94. The system of any one of embodiments 37-93, further comprising a reaction zone, wherein the reaction zone is configured into at least two separate regions comprising one region configured to perform liquid-phase PCR and another region configured to detect an amplification product captured on a capture surface.

95. The system of embodiment 94, wherein the capture surface is modified with a linker layer.

96. The system of embodiment 95, wherein the linker layer is configured to bind to a double or single stranded DNA or RNA strand via the 3′ or the 5′ end.

97. The system of any one of embodiments 95-96, wherein the linker layer comprises silane or small chemical molecules with one or more reactive functional chemical terminal groups.

98. The system of any one of embodiments 95-97, wherein the linker layer comprises single-stranded DNA or RNA bound to the silane or small chemical molecules.

99. The system of any one of embodiments 95-98, wherein the linker layer comprises one or more polymers.

100. The system of embodiment 99, wherein the polymer is a form of dextran, carboxymethyl dextran, chitosan, polyaniline, PEG, PLL-PEG, PLL-g-PEG, PLA-PEG-PLL, or a combination thereof.

101. The system of any one of embodiments 95-100, wherein the linker layer comprises single-stranded DNA or RNA bound to the polymer.

102. The system of any one of embodiments 95-101, wherein the linker layer comprises microparticles and/or nanoparticles.

103. The system of embodiment 102, wherein the microparticles and/or nanoparticles are composed of metal, semiconductor, compound semiconductor, polymer, plastic, oxide, glass, or a combination thereof.

104. The system of any one of embodiments 102-103, wherein the microparticles and/or nanoparticles are configured to bind to silane and/or small chemical molecules of the linker layer.

105. The system of any one of embodiments 102-104, wherein the microparticles and/or nanoparticles comprises a surface, and wherein the surface of the microparticles and/or nanoparticles is at least partially modified with silane and/or small chemical molecules having reactive functional chemical terminal groups to bind with the silane and/or chemical molecules of the linker layer.

106. The system of any one of embodiments 102-105, wherein the microparticles and/or nanoparticles are configured to bind to the DNA or RNA strands of the linker layer.

107. The system of any one of embodiments 102-106, wherein one or more primer strands required for the PCR reaction to amplify a particular DNA/RNA target is chemically or physically bound to the linker layer of any one of embodiments 94-106.

108. The system of embodiment 107, wherein the one or more primer strands comprise forward primer strands or reverse primer strands for a particular DNA/RNA target.

109. The system of any one of embodiments 107-108, wherein the one or more primer strands comprise one set of primer strands for a particular DNA/RNA target, and wherein the one set of primer strands, either the forward or reverse strand, is bound to the linker layer.

110. The system of embodiment 109, wherein a complementary set of primer strands to the one set of primer strands, is present in the liquid sample.

111. The system of any one of embodiments 108-110, wherein both the forward and reverse primer strands for a particular DNA/RNA target are bound to the linker layer.

112. The system of any one of embodiments 37-111, wherein the reader device is a desktop or portable device configured to receive said test cartridge, to perform liquid-phase PCR, and to detect an amplification product.

113. The system of embodiment 112, wherein the reader device is further configured to provide energy, from the energy source, for heating and/or cooling one or more reaction zones on the test cartridge.

114. The system of any one of embodiments 112-113, wherein the reader device is further configured to monitor the temperature, with the thermal sensor, of one or more reaction zones on the test cartridge.

115. The system of any one of embodiments 112-114, wherein the reader device is further configured to adjust the energy output of the energy source to one or more reaction zones on the test cartridge, based on the readings of the thermal sensor, to maintain a selected temperature.

116. The system of any one of embodiments 112-115, wherein the reader device is further configured to activate and/or deactivate a supplementary heating and cooling devices to adjust the temperature of one or more reaction zones on the test cartridge.

117. The system of any one of embodiments 112-116, wherein the reader device is further configured to excite one or more reaction zones on the test cartridge with light of one or more excitation wavelengths using the excitation source.

118. The system of any one of embodiments 112-117, wherein the reader device is further configured to detect and measure the light emitted from one or more reaction zones on the test cartridge, with a light sensor, and convert the readings into one or more output signals.

119. The system of any one of embodiments 112-118, wherein the reader device is further configured to display the one or more output signals on the reader.

120. The system of any one of embodiments 112-119, wherein the reader device is further configured to display or transmit the one or more output signals on another device via a wired or wireless connection.

121. A method for amplifying and detecting nucleic acids on a point-of-care system, the method comprising: providing the point-of-care system of any one of embodiments 37-120; receiving a liquid sample containing PCR components at the test cartridge; modulating an energy output of the energy source to alternatively heat and cool the liquid sample to amplify nucleic acids; amplifying sample nucleic acids in the liquid sample; and measuring or detecting an amplification product using the reader device.

122. The method of embodiment 121, further comprising dispensing a liquid sample containing PCR components, including at least one target-specific primer, and target DNA or RNA into a reaction zone.

123. The method of any one of embodiments 121-122, further comprising measuring a baseline temperature of the liquid sample with a thermal sensor.

124. The method of any one of embodiments 121-123, further comprising monitoring the temperature of the heat generation layer and/or liquid sample with the thermal sensor.

125. The method of any one of embodiments 121-124, further comprising adjusting the energy output of the energy source, based on measurements from the thermal sensor, to reach and maintain the temperature of the liquid sample at the optimal denaturing temperature of the target DNA for the duration of an initial denaturing period.

126. The method of embodiment 125, further comprising allowing the initial denaturing step to continue for a preset duration such that the target DNA in the sample is fully denatured.

127. The method of any one of embodiments 121-126, further comprising reducing the energy output of the energy source until the temperature of the liquid sample reaches an optimal primer annealing temperature, as measured by the thermal sensor.

128. The method of embodiment 127, further comprising adjusting the energy output from the energy source, based on measurements from the thermal sensor, to reach and maintain the temperature of the liquid sample at the optimal primer annealing temperature.

129. The method of any one of embodiments 127-128, further comprising allowing primer annealing to continue for a preset duration such that both the forward and reverse primers fully hybridize to the denatured target DNA strands.

130. The method of any one of embodiments 127-129, further comprising increasing the energy output from the energy source until the temperature of the liquid sample reaches the optimal primer extension temperature, as measured by the thermal sensor.

131. The method of embodiment 130, further comprising adjusting the energy output from the energy source, based on measurements from the thermal sensor, to reach and maintain the temperature of the liquid sample at the optimal primer extension temperature.

132. The method of any one of embodiments 130-131, further comprising allowing primer extension to continue for a preset duration such that the target DNA strand is extended with free nucleotides or free nucleotides labeled with one or more molecules.

133. The method of any one of embodiments 121-132, further comprising repeating primer annealing and primer extensions for a desired number of cycles by adjusting energy output of the energy source and monitoring the temperature of the liquid sample with the thermal sensor.

134. The method of any one of embodiments 121-133, further comprising deactivating the energy source to let the temperature of the liquid sample to return to a preset lower temperature while monitoring the temperature of the liquid sample with the thermal sensor.

135. The method of any one of embodiments 121-133, further comprising measuring fluorescence output from the liquid sample by exciting the sample with an excitation source and measuring the resulting emission with a light sensor having the appropriate filter lens or lenses.

136. The method of any one of embodiments 121-135, further comprising performing a final denaturing step prior to measuring fluorescence output.

137. The method of embodiment 136, wherein prior to measuring the fluorescence output, the method further comprises: dispensing primers tagged with a fluorescent and a quencher molecule into the liquid sample; increasing the temperature of the liquid sample to the denaturing temperature of the target DNA for a preset duration; decreasing the temperature of the liquid sample to a primer annealing temperature of the tagged primers for a preset duration to allow the tagged primers to bind with the amplified target DNA in the liquid sample; and decreasing the temperature of the liquid sample to allow for optimal fluorescence detection.

138. The method of any one of embodiments 121-137, wherein the primers contain a fluorescent dye molecule attached to the 5′ or 3′ prime terminal end but not both.

139. The method of any one of embodiments 121-138, wherein the primers contain a quencher molecule attached to 3′ or 5′ prime terminal end but not both.

140. The method of any one of embodiments 121-139, wherein the primers form a hairpin loop structure when not bound to target amplified DNA, such that fluorescence from the fluorescent molecule is quenched by the quencher molecule.

141. The method of any one of embodiments 121-140, wherein the primers comprise a nucleotide sequence which is complementary to at least a portion of one of the denatured strands of amplified target DNA.

142. The method of any one of embodiments 121-141, wherein the primers elongate and hybridize to the denatured target amplified DNA during the primer anneal step, such that fluorescence from the fluorescent molecule is not quenched by the quencher molecule.

143. A method for performing isothermal PCR reactions, using the system of embodiments 37-120, the method comprising: dispensing a liquid sample containing components for isothermal PCR, comprising recombinase polymerase reaction, loop-mediated isothermal PCR, strand displacement amplification, helicase-dependent amplification, or nicking enzyme amplification, into the sample confinement layer and/or reaction zone(s); performing isothermal amplification for a preset duration; capturing amplified target DNA of the PCR reaction product; and detecting the captured amplified target DNA of the PCR reaction product.

144. The method of embodiment 143, further comprising measuring a baseline temperature of the liquid sample with the thermal sensor.

145. The method of any one of embodiments 143-144, further comprising monitoring the temperature of the heat generation layer and/or liquid sample with the thermal sensor.

146. The method of any one of embodiments 143-145, further comprising adjusting the energy output of the energy source, based on measurements from the thermal sensor, to reach and maintain the temperature of the liquid sample at the optimal denaturing temperature of the target DNA for the duration of the initial denaturing period.

147. The method of any one of embodiments 143-146, further comprising allowing the initial denaturing step to continue for a preset duration such that the target double-stranded DNA in the sample is fully denatured.

148. The method of any one of embodiments 143-147, further comprising reducing the energy output of the energy source until the temperature of the liquid sample reaches the optimal temperature for primal annealing and isothermal amplification, as measured by the thermal sensor.

149. The method of any one of embodiments 143-148, further comprising adjusting the energy output from the energy source, based on measurements from the thermal sensor, to reach and maintain the temperature of the liquid sample at the optimal temperature for isothermal amplification for the duration of the amplification step.

150. The method of any one of embodiments 143-149, further comprising deactivating the energy source to let the temperature of the liquid sample to return to a preset lower temperature while monitoring the temperature of the liquid sample with the thermal sensor.

Some aspects described herein further relate to the following additional embodiments, referred to as alternatives:

1. A PCR temperature cycling system comprising: a reader comprising: an energy source; a thermal sensor; a supplementary heating device; a supplementary cooling device; an excitation source; a light sensor; a power supply; control and/or I/O circuitry; a display; an opening to receive a test cartridge; and any subcombinations of the above features; a test cartridge, with at least one reaction zone comprising; a substrate; a 3D pattern layer; a heat generation layer; a thermal conduction layer; a passivation layer; a liquid sample; a sample confinement layer; a capping and/or encapsulation layer; and any subcombinations of the above features.

2. The PCR temperature cycling system of alternative 1, wherein the energy source is a light source.

3. The PCR temperature cycling system of alternative 2, wherein the light source is a light emitting diode, an array of light emitting diodes, a laser diode, an array of laser diodes, DPSS laser, an array of DPSS lasers, at least one focusing lens, at least one collimating lens or any combination thereof.

4. The PCR temperature cycling system of alternative 1, wherein the energy source is a voltage and/or current source.

5. The PCR temperature cycling system of alternative 1, wherein the thermal sensor comprises one or more non-contact infrared detector(s).

6. The PCR temperature cycling system of alternative 5, wherein the infrared detector is a charge coupled device (CCD), complementary metal-oxide semiconductor device (CMOS), photovoltaic device, photodiode device, photoconductor device, thermopile device, bolometer device or any combination thereof.

7. The PCR temperature cycling system of alternative 5, wherein the thermal sensor is positioned to be under the liquid sample.

8. The PCR temperature cycling system of alternative 5, wherein the thermal sensor is positioned to be above the liquid sample.

9. The PCR temperature cycling system of alternative 1, wherein the thermal sensor comprises one or more contact temperature sensors.

10. The PCR temperature cycling system of alternative 9, wherein the contact temperature sensor is a thermocouple, resistance temperature detector, thermistor or any combination thereof.

11. The PCR temperature cycling system of alternative 9, wherein the thermal sensor is positioned to be in contact with the liquid sample.

12. The PCR temperature cycling system of alternative 11, wherein one or more thermal sensor(s) is (are) placed inside the sample confinement layer and in contact with the liquid sample.

13. The PCR temperature cycling system of alternative 11, wherein the thermal sensor is configured as a resistance temperature device or thermistor patterned and/or fabricated in close proximity to the heat generation layer.

14. The PCR temperature cycling system of alternative 1, wherein the supplementary heating device is a thermoelectric device, heat block, resistive heater, printed circuit board heater, flexible circuit heater or any combination thereof.

15. The PCR temperature cycling system of alternative 1, wherein the supplementary cooling device is a heatsink, fan, thermoelectric device, Peltier cooler or any combination thereof.

16. The PCR temperature cycling system of alternative 1, wherein the excitation source is a light emitting diode, an array of light emitting diodes, a laser diode, an array of laser diodes, DPSS laser, an array of DPSS lasers, at least one focusing lens, at least one collimating lens and/or any combination thereof.

17. The PCR temperature cycling system of alternative 1, wherein the excitation source is the same as the energy source.

18. The PCR temperature cycling system of alternative 1, wherein the light sensor is a charge coupled device (CCD), complementary metal-oxide semiconductor device (CMOS), photovoltaic device, photodiode device, photoconductor device or any combination thereof.

19. The PCR temperature cycling system of alternative 1, wherein the reader is a desktop or portable device configured to: receive a test cartridge; provide energy, from the energy source, for heating and/or cooling one or more reaction zones on the test cartridge; monitor the temperature, with the thermal sensor, of one or more reaction zones on the test cartridge; adjust the energy output of the energy source to one or more reaction zones on the test cartridge, based on the readings of the thermal sensor, to maintain the selected temperature; activate and/or deactivate the supplementary heating and cooling devices to adjust the temperature of one or more reaction zones on the test cartridge; excite one or more reaction zones on the test cartridge with light of one or more excitation wavelengths using the excitation source; detect and measure the light emitted from one or more reaction zones on the test cartridge, with the light sensor, and convert the readings into one or more output signal(s); display the output signal(s) on the reader; display or transmit the output signal(s) on another device via a wired or wireless connection; and any subcombinations of the above actions.

20. The PCR temperature cycling system of alternative 1, wherein the PCR reaction is configured to occur within the liquid sample and more specifically anywhere and/or everywhere within the bulk of the liquid sample.

21. The PCR temperature cycling system of alternative 1, wherein the reaction zone is configured as one or an array of well, hole, groove and/or trench structures.

22. The PCR temperature cycling system of alternative 1, wherein the reaction zone is configured as one channel or an array of channels.

23. The PCR temperature cycling system of alternative 20, wherein the confining boundaries of the well structure(s) comprises the substrate on the bottom side, the sample confinement layer on the sides, and the capping and/or encapsulation layer on the top side.

24. The PCR temperature cycling system of alternative 22, wherein the channel(s) is (are) confined only on the top side and the bottom side by a substrate.

25. The PCR temperature cycling system of alternative 24, wherein the substrate on the top side is the same as the substrate on the bottom side.

26. The PCR temperature cycling system of alternative 24, wherein the substrate on the top side is different from the substrate on the bottom side.

27. The PCR temperature cycling system of alternative 24, wherein the substrate is configured as the base for coatings, depositions, and/or fabrications of one or more 3D pattern layer(s), heat generation layer(s), thermal conduction layer(s), passivation layer(s), sample confinement layer(s), capping or encapsulation layer(s) or any combination thereof.

28. The PCR temperature cycling system of alternative 28, wherein the substrate comprises semiconductor, metal, FR-4, polymer, plastic, epoxy, resin, glass, silicone, rubber, a track-etched membrane or any combination thereof.

29. The PCR temperature cycling system of alternative 28, wherein the material(s) comprising the substrate is (are) at least partially transparent in the wavelength range between 400 nanometers to 1 micrometer.

30. The PCR temperature cycling system of alternative 28, wherein the material(s) comprising the substrate is (are) at least partially transparent in the mid to long infrared spectrum, preferably in the wavelength range between 5 micrometers to 13 micrometers.

31. The PCR temperature cycling system of alternative 1, wherein the 3D pattern layer is positioned under the heat generation layer and is configured to increase the surface area of the heat generation layer and/or increase the height of the heat generation layer so as to reduce the length that the reactants must diffuse to reach the heat generation layer.

32. The PCR temperature cycling system of alternative 31, wherein the 3D pattern layer comprises polymer, plastic, silicone, rubber, glass, metal-oxide, semiconductor-oxide or any combination thereof.

33. The PCR temperature cycling system of alternative 31, wherein the 3D pattern layer is a planar layer.

34. The PCR temperature cycling system of alternative 31, wherein the 3D pattern layer structure comprises patterned and/or deposited features and/or structures.

35. The PCR temperature cycling system of alternative 34, wherein the features and/or structures comprises one or arrays of pillars, lines, line and space gratings, pyramids, triangles, trenches, spheres or any combination thereof.

36. The PCR temperature cycling system of alternative 34, wherein the 3D patterns' features/structures are deposited and/or fabricated by photolithography, fused deposition modeling 3D printing, stereolithography 3D printing, selective laser sintering 3D printing, inkjet printing, molding, microarray printing/blotting/spotting or any combination thereof.

37. The PCR temperature cycling system of alternative 31, wherein the material(s) comprising the substrate is (are) at least partially transparent in the wavelength range between 400 nanometers to 1 micrometer.

38. The PCR temperature cycling system of alternative 31, wherein the material(s) comprising the substrate is (are) at least partially transparent in the mid to long infrared spectrum, preferably in the wavelength range between 5 micrometers to 13 micrometers.

39. The PCR temperature cycling system of alternative 1, wherein the heat generation layer is positioned on top of the 3D pattern layer.

40. The PCR temperature cycling system of alternative 1, wherein the heat generation layer is a light absorbing layer.

41. The PCR temperature cycling system of alternative 40, wherein the light absorbing layer is configured to absorb the light energy input from the energy source and transform it into thermal energy.

42. The PCR temperature cycling system of alternative 41, wherein the thermal energy is produced in the light absorbing layer is proportional to the power of the light energy output from the energy source.

43. The PCR temperature cycling system of alternative 40, wherein the light absorbing layer comprises pigment, dye, semiconductor, compound semiconductor, carbon nanotubes, fullerenes, graphene, graphene-oxide, metal-oxide, semiconductor-oxide, polymer, plastic, metal, metal-alloy or any combination thereof.

44. The PCR temperature cycling system of alternative 43, wherein the light absorbing layer preferably comprises germanium.

45. The PCR temperature cycling system of alternative 43, wherein the light absorbing layer preferably comprises polyimide.

46. The PCR temperature cycling system of alternative 43, wherein the light absorbing layer preferably comprises pigment or dye or a pigmented or dyed plastic film or sheet.

47. The PCR temperature cycling system of alternative 40, wherein the light absorbing layer comprises nanoparticles and/or microparticles composed of metal, semiconductor, compound semiconductor, polymer, plastic, oxide, glass or any combination thereof.

48. The PCR temperature cycling system of alternative 46, wherein the nanoparticles and/or microparticles are infused with light absorbing materials of alternative 43.

49. The PCR temperature cycling system of alternative 46, wherein the nanoparticles and/or microparticles are capped with light absorbing materials of alternative 43.

50. The PCR temperature cycling system of alternative 1, wherein the heat generation layer is a resistive heater layer.

51. The PCR temperature cycling system of alternative 49, wherein the resistive heater layer is configured as one or more traces and/or circuits to dissipate or absorb the voltage and/or current energy input from the energy source and transform it into thermal energy.

52. The PCR temperature cycling system of alternative 50, wherein thermal energy is produced in the trace(s) and/or circuit(s) of the resistive heater layer, which is proportional to the resistance of the trace(s) and/or circuit(s) of the resistive heater layer and the current flowing from the energy source and into the trace(s) and/or circuit(s) of the resistive heater layer.

53. The PCR temperature cycling system of alternative 49, wherein the trace(s) and/or circuit(s) of the resistive heater layer comprises semiconductor, compound semiconductor, carbon nanotubes, fullerenes, graphene, graphene-oxide, metal-oxide, semiconductor-oxide, metal, metal-alloy or any combination thereof.

54. The PCR temperature cycling system of alternative 1, wherein the thermal conduction layer is the same as the heat generation layer.

55. The PCR temperature cycling system of alternative 1, wherein the thermal conduction layer is a separate layer positioned on top of the heat generation layer.

56. The PCR temperature cycling system of alternative 54, wherein the thermal conduction layer is configured to facilitate heat transfer from the heat generation layer to the liquid sample and/or heat transfer from the liquid sample to the heat generation layer.

57. The PCR temperature cycling system of alternative 1, wherein the thermal conduction layer is a separate layer positioned on top of the substrate or 3D pattern layer.

58. The PCR temperature cycling system of alternative 56, wherein the thermal conduction layer is configured for heat transfer from the supplementary heating device to the liquid sample and/or heat transfer from the liquid sample to the supplementary cooling device.

59. The PCR temperature cycling system of alternatives 54 and 56, wherein the thermal conduction layer comprises metal, metal-alloy, semiconductor, compound semiconductor, graphene, carbon nanotubes, fullerenes, nanoparticles, microparticles, metal-oxide, semiconductor-oxide or any combination thereof.

60. The PCR temperature cycling system of alternative 1, wherein the passivation layer is the same as the heat generation layer and/or thermal conduction layers.

61. The PCR temperature cycling system of alternative 1, wherein the passivation layer is a separate layer positioned on top of the heat generation layer.

62. The PCR temperature cycling system of alternative 1, wherein the passivation layer is a separate layer positioned on top of the thermal conduction layer.

63. The PCR temperature cycling system of alternatives 60 and 61, wherein the passivation layer is configured to form the interface to the liquid sample and isolate the heat generation and/or thermal conduction layers from liquid sample.

64. The PCR temperature cycling system of alternative 62, wherein the passivation layer comprises metal-oxide, semiconductor-oxide, glass, photoresist, plastic, polymer, semiconductor, metal, metal-alloy or any combination thereof.

65. The PCR temperature cycling system of alternative 62, wherein the surface of the passivation layer is further coated or modified with chemical molecules, silane, protein, nucleic acids or any combination thereof.

66. The PCR temperature cycling system of alternative 1, wherein the liquid sample comprises target DNA, polymerase, DNase inhibitor, forward primer sequence strands, reverse primer sequence strands, free unlabeled nucleotides, free nucleotides labeled with one or more molecules, water, buffer salts, metal ions or any combination thereof.

67. The PCR temperature cycling system of alternative 1, wherein the liquid sample comprises target RNA or mRNA, reverse transcriptase, polymerase, RNase inhibitor, forward primer sequence strands, reverse primer sequence strands, free unlabeled nucleotides, free nucleotides labeled with one or more molecules, water, buffer salts, metal ions or any combination thereof.

68. The PCR temperature cycling system of alternative 20, wherein the sample confinement layer comprises the well, hole, groove and/or trench structure(s).

69. The PCR temperature cycling system of alternative 67, wherein well, hole, groove and/or trench structure(s) are fabricated from metal-oxide, semiconductor-oxide, metal, metal-alloy, glass, plastic, polymer, photoresist, silicone, rubber or any combination thereof.

70. The PCR temperature cycling system of alternative 67, wherein the surface of the sample confinement layer is coated with thermally conductive material(s) such as metal, metal-alloy, semiconductor, compound semiconductor, graphene, fullerenes, carbon nanotubes, nanoparticles, microparticles or any combination thereof.

71. The PCR temperature cycling system of alternative 69, wherein the coating of thermally conductive material, on the surface of the sample confinement layer, is further coated with a passivating material such as metal-oxide, semiconductor-oxide, glass, photoresist, plastic, polymer, semiconductor, metal, metal-alloy or any combination thereof.

72. The PCR temperature cycling system of alternative 1, wherein the capping and/or encapsulation layer is configured to prevent evaporation of the liquid sample.

73. The PCR temperature cycling system of alternative 71, wherein the capping and/or encapsulation layer is a layer of oil on top of the liquid sample.

74. The PCR temperature cycling system of alternative 71, wherein the capping and/or encapsulation layer is a film of plastic and/or glass enclosing the top surfaces of the well, hole, groove, and/or trench structure(s) of the sample confinement layer.

75. The PCR temperature cycling system of alternative 71, wherein the capping and/or encapsulation layer is a substrate of alternative 0.

76. A method for performing PCR reactions, utilizing the PCR temperature cycling system of alternative 1, comprising: dispensing a liquid sample containing standard PCR components, including at least one target-specific primer, and target DNA or RNA into the sample confinement layer and/or reaction zone(s); measuring the baseline temperature of the liquid sample with the thermal sensor; monitoring the temperature of the heat generation layer and/or liquid sample with the thermal sensor; adjusting the energy output of the energy source, based on measurements from the thermal sensor, to reach and maintain the temperature of the liquid sample at the optimal denaturing temperature of the target DNA for the duration of the initial denaturing period; allowing the initial denaturing step to continue for a preset duration such that the target double-stranded DNA in the sample is fully denatured; reducing the energy output of the energy source until the temperature of the liquid sample reaches the optimal primer annealing temperature, as measured by the thermal sensor; adjusting the energy output from the energy source, based on measurements from the thermal sensor, to reach and maintain the temperature of the liquid sample at the optimal primer annealing temperature for the duration of the primer annealing step; allowing the primer annealing step to continue for a preset duration such that both the forward and reverse primers fully hybridize to the denatured target DNA strands; increasing the energy output from the energy source until the temperature of the liquid sample reaches the optimal primer extension temperature, as measured by the thermal sensor; adjusting the energy output from the energy source, based on measurements from the thermal sensor, to reach and maintain the temperature of the liquid sample at the optimal primer extension temperature for the duration of the primer extension step; allowing the primer extension step to continue for a preset duration such that the target DNA strand is extended with free nucleotides or free nucleotides labeled with one or more molecules; repeating the primer anneal and primer extension steps for a desired number of cycles by adjusting energy output of the energy source and monitoring the temperature of the liquid sample with the thermal sensor; deactivating the energy source to let the temperature of the liquid sample to return to a preset lower temperature while monitoring the temperature of the liquid sample with the thermal sensor; and measuring fluorescence output from the liquid sample by exciting the sample with an excitation source and measuring the resulting emission with a light sensor having the appropriate filter lens or lenses.

77. The method of performing PCR reactions of alternative 76, where a final denaturing step is performed prior to measuring fluorescence output.

78. The method of performing PCR reactions of alternative 76, where prior to measuring the fluorescence output: primers tagged with a fluorescent and a quencher molecule are dispensed into the liquid sample; the temperature of the liquid sample is raised to the denaturing temperature of the target DNA for a preset duration; the temperature of the liquid sample is reduced to the primer annealing temperature of the tagged primers for a preset duration to allow the tagged primers to bind with the amplified target DNA in the liquid sample; and the temperature of the liquid sample is reduced to allow for optimal fluorescence detection.

79. The method of alternative 78, wherein the primers contain a fluorescent dye molecule attached to the 5′ or 3′ prime terminal end but not both.

80. The method of alternative 78, wherein the primers contain a quencher molecule attached to 3′ or 5′ prime terminal end but not both.

81. The method of alternative 78, wherein the primers form a hairpin loop structure when not bound to target amplified DNA, such that fluorescence from the fluorescent molecule is quenched by the quencher molecule.

82. The method of alternative 78, wherein the tagged primers have a nucleotide sequence which is complementary to at least a portion of one of the denatured strands of amplified target DNA.

83. The method of alternative 78, wherein the primers elongate and hybridize to the denatured target amplified DNA during the primer anneal step, such that fluorescence from the fluorescent molecule is not quenched by the quencher molecule.

84. The PCR temperature cycling system of alternative 1, wherein a PCR reaction is configured to occur at or in close proximity of the surface of the reaction zone(s).

85. The PCR temperature cycling system of alternative 1, wherein the at least one reaction zone is configured into at least two separate regions with one region where liquid-phase PCR occurs and another region where amplified DNA of PCR product is captured on a surface and detected.

86. The PCR temperature cycling system of alternative 84 and 85, wherein the surface of the at least one reaction zone is the passivation layer, thermal conduction layer, heat generation layer, or 3D pattern layer or sample confinement layer.

87. The PCR temperature cycling system of alternative 86, wherein the surface is modified with a linker layer.

88. The PCR temperature cycling system of alternative 87, wherein the linker layer is configured to bind to a double or single stranded DNA or RNA strand via the 3′ or the 5′ end.

89. The PCR temperature cycling system of alternative 87, wherein the linker layer comprises silane and/or small chemical molecules with one or more reactive functional chemical terminal group(s).

90. The PCR temperature cycling system of alternative 89, wherein the linker layer further comprises single-stranded DNA or RNA bound to the silane and/or small chemical molecules.

91. The PCR temperature cycling system of alternative 89, wherein the linker layer further comprises one or more polymers.

92. The PCR temperature cycling system of alternative 91, wherein the polymer is a form of dextran, carboxymethyl dextran, chitosan, polyaniline, PEG, PLL-PEG, PLL-g-PEG, PLA-PEG-PLL or any combination thereof.

93. The PCR temperature cycling system of alternative 91, wherein the linker comprises single-stranded DNA or RNA bound to the polymer.

94. The PCR temperature cycling system of alternative 87, wherein the linker layer comprises microparticles and/or nanoparticles.

95. The PCR temperature cycling system of alternative 94, wherein the particles are composed of metal, semiconductor, compound semiconductor, polymer, plastic, oxide, glass or any combination thereof.

96. The PCR temperature cycling system of alternative 94, wherein the particles are configured to bind to silane and/or small chemical molecules of the linker layer of alternative 89.

97. The PCR temperature cycling system of alternative 96, wherein the surface of the particles is at least partially modified with silane and/or small chemical molecules having reactive functional chemical terminal groups to bind with the silane and/or chemical molecules of the linker layer of alternative 89.

98. The PCR temperature cycling system of alternative 94, wherein the particles are configured to bind to the polymer of the linker layer of alternative 91.

99. The PCR temperature cycling system of alternative 98, wherein the surface of the particles is at least partially modified with silane and/or small chemical molecules having reactive functional chemical terminal groups to bind with the polymer of the linker layer of alternative 91.

100. The PCR temperature cycling system of alternative 94, wherein the particles are configured to bind to the DNA or RNA strands of the linker layer of alternative 90.

101. The PCR temperature cycling system of alternative 94, wherein the particles are configured to bind to the DNA or RNA strands of the linker layer of alternative 93.

102. The PCR temperature cycling system of alternatives 100 and 101, wherein the particles are at least partially modified with single stranded DNA or RNA at least partially complementary to the strands of alternatives 90 and 93.

103. The PCR temperature cycling system of alternative 87, wherein one or more primer(s) strand(s) required for the PCR reaction(s) to amplify a particular DNA/RNA target is(are) chemically or physically bound to the linker layer.

104. The PCR temperature cycling system of alternative 103, wherein the primer strands comprise the forward primer strands and/or the reverse primer strands for a particular DNA/RNA target.

105. The PCR temperature cycling system of alternative 84, wherein one set of primer strands for a particular DNA/RNA target, either the forward or reverse strand, is bound to the linker layer.

106. The PCR temperature cycling system of alternative 105, wherein one set of primer strands, different from the primer strand of alternative 105, is present in the liquid sample.

107. The PCR temperature cycling system of alternative 84, wherein both the forward and reverse primer strands for a particular DNA/RNA target are bound to the linker layer.

108. The PCR temperature cycling system of alternative 85, which are configured for capture and detection of amplified DNA, wherein neither the forward nor reverse primer strands are bound to the surface of the reaction zone(s).

109. The PCR temperature cycling system of alternative 108, wherein single-stranded DNA strands or probes which are at least partially complementary to the amplified target DNA in PCR reaction product, but may or may not share sequences with the forward nor reverse primers, are bound to the linker layer on the surface of the reaction zone(s).

110. A method for performing PCR reactions, utilizing the PCR temperature cycling system of alternative 1 with the reaction zone of alternative 85, comprising: dispensing a liquid sample containing standard PCR components, including at least one target-specific primer, and target DNA or RNA into the sample confinement layer and/or reaction zone(s); measuring the baseline temperature of the liquid sample with the thermal sensor; monitoring the temperature of the heat generation layer and/or liquid sample with the thermal sensor; adjusting the energy output of the energy source, based on measurements from the thermal sensor, to reach and maintain the temperature of the liquid sample at the optimal denaturing temperature of the target DNA for the duration of the initial denaturing period; allowing the initial denaturing step to continue for a preset duration such that the target double-stranded DNA in the sample is fully denatured; reducing the energy output of the energy source until the temperature of the liquid sample reaches the optimal primer annealing temperature, as measured by the thermal sensor; adjusting the energy output from the energy source, based on measurements from the thermal sensor, to reach and maintain the temperature of the liquid sample at the optimal primer annealing temperature for the duration of the primer annealing step; allowing the primer annealing step to continue for a preset duration such that both the forward and reverse primers fully hybridize to the denatured target DNA strands; increasing the energy output from the energy source until the temperature of the liquid sample reaches the optimal primer extension temperature, as measured by the thermal sensor; adjusting the energy output from the energy source, based on measurements from the thermal sensor, to reach and maintain the temperature of the liquid sample at the optimal primer extension temperature for the duration of the primer extension step; allowing the primer extension step to continue for a preset duration such that the target DNA strand is extended with free nucleotides or free nucleotides labeled with one or more fluorescent molecules; repeating the primer anneal and primer extension steps for a desired number of cycles by adjusting energy output of the energy source and monitoring the temperature of the liquid sample with the thermal sensor; deactivating the energy source to let the temperature of the liquid sample to return to a preset lower temperature while monitoring the temperature of the liquid sample with the thermal sensor; allowing the liquid sample to enter the region of the reaction zone configured for capture and detection of amplified target DNA of the PCR reaction product; activating the energy source and adjusting the energy output from the energy source, based on measurements from the thermal sensor, to reach and maintain the temperature of the liquid sample at the optimal denaturing temperature of the target DNA for a preset duration such that the amplified target DNA is fully denatured; allowing the denatured amplified target DNA strands to bind to the single-stranded DNA probes of the reaction zone(s) of alternative 109; washing away the remaining PCR product to a waste chamber and flushing the reaction zone(s) with a buffer solution; exciting the reaction zone(s) of alternative 109, which contain bound amplified target DNA, with the excitation source; and measuring the resulting fluorescence emission with a light sensor having the appropriate filter lens or lenses.

111. A method for performing isothermal PCR reactions, utilizing the PCR temperature cycling system of alternative 1: dispensing a liquid sample containing standard components for isothermal PCR, which may include recombinase polymerase reaction, loop-mediated isothermal PCR, strand displacement amplification, helicase-dependent amplification, or nicking enzyme amplification, into the sample confinement layer and/or reaction zone(s); measuring the baseline temperature of the liquid sample with the thermal sensor; monitoring the temperature of the heat generation layer and/or liquid sample with the thermal sensor; adjusting the energy output of the energy source, based on measurements from the thermal sensor, to reach and maintain the temperature of the liquid sample at the optimal denaturing temperature of the target DNA for the duration of the initial denaturing period; allowing the initial denaturing step to continue for a preset duration such that the target double-stranded DNA in the sample is fully denatured; reducing the energy output of the energy source until the temperature of the liquid sample reaches the optimal temperature for primal annealing and isothermal amplification, as measured by the thermal sensor; adjusting the energy output from the energy source, based on measurements from the thermal sensor, to reach and maintain the temperature of the liquid sample at the optimal temperature for isothermal amplification for the duration of the amplification step; allowing the isothermal amplification to occur for a preset duration; deactivating the energy source to let the temperature of the liquid sample to return to a preset lower temperature while monitoring the temperature of the liquid sample with the thermal sensor; and allowing the liquid sample to enter the region of the reaction zone configured for capture and detection of amplified target DNA of the PCR reaction product.

112. A nanoparticle assay system comprising: a sample potentially containing one or more analytes of interest; an assay cartridge comprising a test region including a nonporous and/or non-membranous surface; a test region containing one or more capture regions; analyte binding capture probe molecules on the surface of the capture region(s) and within the test region; nanoparticles conjugated with analyte binding capture probe molecules; a source of radiation wherein radiation excites the nanoparticles to produce a measurable response; a ultraviolet, visible and/or thermal radiation detector; and any subcombinations of the above features.

113. The system of alternative 112, where the test region comprises a nonporous and/or non-membranous surface comprising polymer, epoxy, plastic, semiconductor, oxide, metal and/or any combination thereof.

114. The system of alternatives 112 or 113, where the surface of the test region is coated with a reflective material, such as silver or aluminum, or a dielectric mirror stack designed to specifically reflect the incident energy, which is coated by a thin layer of dielectric.

115. The system of any one of alternatives 112 to 114, where the surface of the test region comprises three dimensional patterned structures comprising polymer, epoxy, plastic, semiconductor, oxide, metal and/or any combination thereof.

116. The system of any one of alternatives 112 to 115, where the analyte binding capture probe molecules are coupled to the surface of the test region by linker molecules.

117. The system of alternative 116, where the linker molecules comprise one or more chemical molecules and/or functional silane wherein one end terminal end of the molecule, or chain of molecules, is bound to the surface of the test region and wherein another terminal end of the molecule, or chain of molecules, comprises a functional group capable of binding capture probe molecules.

118. The system of any one of alternatives 112 to 117, wherein the assay cartridge comprises multiple capture regions within the test region, the multiple capture regions coated with the same or different capture probes.

119. The system of any one of alternatives 112 to 118, where the nanoparticles comprise one or more layers of gold, silver, carbon, platinum, polymer, plastic, oxide, iron and/or any combination thereof.

120. The system of any one of alternatives 112 to 119, where the geometry of the nanoparticles comprises spheres, cylinders, rods, core-shell particles, urchins, stars, plates, cubes, porphysomes and/or any combination thereof.

121. The system of any one of alternatives 112 to 120, where analyte binding capture probe molecules are coupled to the surface of nanoparticles by linker molecules.

122. The system of any one of alternatives 112 to 121, where the linker molecules comprise one or more chemical molecules and/or functional silane wherein one terminal end of the molecule, or chain of molecules, is bound to the surface of the nanoparticle and another terminal end of the molecule, or chain of molecules, comprise a functional group capable of binding analyte binding capture probe molecules.

123. The nanoparticles assay system of any one of alternatives 112 to 122, where the capture probe molecules comprise chemical molecules, antibody, enzyme, protein, oligonucleotide, single-stranded DNA, double-stranded DNA, aptamer, DNAzyme, aptazyme, a synthetic molecule capable of binding target analyte in sample and/or any combination thereof.

124. The system of any one of alternatives 112 to 123, where the analyte or analytes of interest comprise oligonucleotides, proteins, antibodies, chemical molecules and/or any combination thereof.

125. The system of any one of alternatives 112 to 124, where the source of radiation comprises a diode laser, DPSS laser, fiber-coupled laser, light-emitting diode and/or any combination thereof.

126. The system of any one of alternatives 112 to 125, where the radiation detector comprises a CMOS or CCD device, a photodiode, an infrared camera module, an infrared-sensitive semiconductor chip or circuit, and/or any combination thereof.

127. The system of any one of alternatives 112 to 126, where the test region comprises a material which is transparent to radiation and does not absorb incident radiation or absorbs a known quantity of incident radiation.

128. The system of alternative 127, where the test region comprises a thin sheet of infrared transparent plastic, semiconductor, metal oxide, chalcogenide, semiconductor oxide and/or any combination thereof.

129. The system of alternative 128, where the surface of the infrared transparent material comprises one or more layers of three dimensional patterned structures made from polymer, epoxy, plastic, semiconductor, oxide, metal and/or any combination thereof.

130. A method of performing a nanoparticle assay using the nanoparticle assay system of any one of alternatives 112 to 129 and/or another nanoparticle assay system, the method comprising: dispensing buffer and/or similar solution to the test region; exposing the test region, before contact with any sample solution, to a source of radiation; measuring a baseline reading with the thermal radiation detector; dispensing the sample, containing analyte/analytes of interest to the test region, wherein the sample is propelled towards the test region by external pressure and/or is manipulated by pipetting; allowing the sample to react with the test region for a duration configured so that present analyte(s) of interest to bind to the test region; washing and/or rinsing the test region with buffer and/or similar solution one or multiple times; dispensing a solution containing nanoparticles conjugated with analyte binding capture probe molecules to the test region and allowing reaction for a pre-set period of time; washing and/or rinsing the test region with buffer and/or similar solution one time or multiple times; exposing the test region to said radiation for a pre-set period of time; detecting the infrared radiation emitted from the test region with the thermal radiation detector; calculating and reporting the concentration of analyte by analyzing the detected thermal radiation signal; and any subcombinations of the above features.

131. The method of alternative 130, where measuring the baseline reading is done without exposing the test region to a source of radiation.

132. The method of alternative 130 or 131, where the test region is not washed before exposing the test region to a solution containing nanoparticles conjugated with analyte binding capture probe molecules.

133. The method of any one of alternatives 130 to 132, where the test region is not washed before exposing the test region to radiation and detecting the thermal radiation signal.

134. The method of any one of alternatives 130 to 133, further comprising removing the bulk of the fluid over the test region prior to exposing the test region to incident energy and measuring the thermal response.

135. A method of performing a nanoparticle assay using the nanoparticle assay system of any one of alternatives 112 to 129 and/or another nanoparticle assay system, the method comprising: dispensing buffer and/or similar solution to the test region; exposing the test region, before contact with any sample solution, to a source of radiation; measuring a baseline reading with the thermal radiation detector; mixing the sample, containing analyte/analytes of interest, with solution containing nanoparticles conjugated with analyte binding capture probe molecules for a pre-set period of time; dispensing the solution containing nanoparticle/analyte complexes to the test region, wherein the sample is propelled towards the test region by external pressure and/or is manipulated by pipetting; allowing the solution to react with the test region for a duration configured so that nanoparticle/analyte complexes to bind to the surface of the capture region(s); washing and/or rinsing the test region with buffer or similar solution one or multiple times; exposing the test region to said radiation for a pre-set period of time; detecting the infrared radiation emitted from the test region with the thermal radiation detector; calculating and reporting the concentration of analyte by analyzing the detected thermal radiation signal; and any subcombinations of the above features.

136. The method of alternative 135, where measuring the baseline reading is without exposing the test region to a source of radiation.

137. The method of c1 alternative 135 or 136, where the test region is not washed before exposing the test region to radiation and detecting the thermal radiation signal.

138. The method of any one of alternatives 135 to 137, further comprising removing the bulk of the fluid over the test region prior to exposing the test region to incident energy and measuring the thermal response.

139. A method of performing a nanoparticle assay using the nanoparticle assay system of any one of alternatives 112 to 129 and/or another nanoparticle assay system, the method comprising: dispensing buffer and/or similar solution to the test region; exposing the test region, before contact with any sample solution, to a source of radiation; measuring a baseline reading with the thermal radiation detector; mixing the sample with capture probe molecules, where the capture probe molecules may be a single type for capture of single target analyte or different types for capture of multiple analytes; dispensing the solution with capture probe molecules bound to target analyte(s) towards the test region; allowing the solution to react with the test region for a pre-set period of time, causing the capture probe/analyte complexes to bind to capture probe molecules on the surface of the capture region; washing and/or rinsing the test region with buffer and/or similar solution one or multiple times; providing nanoparticles modified with capture probe molecules which bind to all of the capture probes attached to the analytes at the surface of the test region, even different capture probes attached to different analytes; dispensing the solution with the nanoparticle/probe complexes toward the test region; allowing the solution to react with the capture probes exposed on the surface of the test region for duration configured so that the nanoparticles bind to the capture probes on the surface; washing and/or rinsing the test region with buffer and/or similar solution one time or multiple times; exposing the test region to incident radiation for a pre-set period of time; detecting the infrared radiation emitted from the test region with the thermal radiation detector; calculating and reporting the concentration of analyte by analyzing the detected thermal radiation signal; and any subcombinations of the above features.

140. The method of alternative 139, where measuring the baseline reading is without exposing the test region to a source of radiation.

141. The method of alternative 139 or 140, where the test region is not washed before dispensing solution with the nanoparticle/probe complexes toward the test region.

142. The method of any one of alternatives 139 to 141, where the test region is not washed before exposing the test region to radiation and detecting the thermal radiation signal.

143. The method of any one of alternatives 139 to 142, further comprising removing the bulk of the fluid over the test region prior to exposing the test region to incident energy and measuring the thermal response.

144. A method of performing a nanoparticle assay using the nanoparticle assay system of any one of alternatives 112 to 129 and/or another nanoparticle assay system, the method comprising: dispensing buffer and/or similar solution to the test region; exposing the test region, before contact with any sample solution, to a source of radiation; measuring a baseline reading with the thermal radiation detector; denaturing the double stranded amplified DNA, wherein the DNA is a product of an amplification process, to separate the DNA into two sets of single stranded DNA, denoted A1 and A2; allowing one set of strands, with sequence A1, to bind to a surface conjugated with single stranded DNA partially or fully complementary to sequence A1; providing a test region with a surface conjugated to single stranded DNA capture probes, with sequence partially complementary to A2; dispensing the remaining separated single stranded DNA, with sequence A2, to the test region and allowing it to hybridize to the capture probes on the surface of the test region; washing and/or rinsing the test region with buffer and/or similar solution one time or multiple times; providing nanoparticles conjugated to single stranded DNA capture probes, with sequence partially complementary to A2, dispensing the solution with the nanoparticle/capture probe complexes to the test region and allowing hybridization to the partially hybridized strands, with sequence A2, exposed on the surface of the test region; washing and/or rinsing the test region with buffer and/or similar solution one time or multiple times; exposing the test region to incident radiation for a pre-set period of time; detecting the infrared radiation emitted from the test region with the thermal radiation detector; calculating and reporting the concentration and/or the presence of any hybridized DNA by analyzing the detected thermal radiation signal; and any subcombinations of the above features.

145. The method of alternative 144, where measuring the baseline reading is without exposing the test region to a source of radiation.

146. The method of alternative 144 or 145, where the test region is not washed before dispensing solution with the nanoparticle/probe complexes toward the test region.

147. The method of any one of alternatives 144 to 146, where the test region is not washed before exposing the test region to radiation and detecting the thermal radiation signal.

148. The method of any one of alternatives 144 to 147, further comprising removing the bulk of the fluid over the test region prior to exposing the test region to incident energy and measuring the thermal response.

149. A method of performing a nanoparticle assay using the nanoparticle assay system of any one of alternatives 112 to 129 and/or another nanoparticle assay system, the method comprising: dispensing buffer and/or similar solution to the test region; exposing the test region, before contact with any sample solution, to a source of radiation; measuring a baseline reading with the thermal radiation detector; providing nanoparticles conjugated to single stranded DNA, with sequence B1; providing a test region with a surface conjugated to single stranded DNA capture probes, with sequence partially or fully complementary to sequence B1; dispensing the solution with the nanoparticle/DNA complexes to the test region and allowing hybridization to single stranded DNA exposed on the surface of the test region; washing and/or rinsing the test region with buffer and/or similar solution one time or multiple times; exposing the test region to incident radiation for a pre-set period of time; detecting the infrared radiation emitted from the test region with the thermal radiation detector; calculating and reporting the concentration and/or the presence of any hybridized DNA by analyzing the detected thermal radiation signal; and any subcombinations of the above features.

150. The method of alternative 149, where measuring the baseline reading is without exposing the test region to a source of radiation.

151. The method of alternative 149 or 150, where the test region is not washed before exposing the test region to radiation and detecting the thermal radiation signal.

152. The method of any one of alternatives 149 to 151, where the bulk of the fluid over the test region is removed prior to exposing the test region to incident energy and measuring the thermal response.

153. A method of measuring the signal in the methods of any one of alternatives 130, 135, 139, 144, and 149, comprising: exposing the test region to incident radiation, periodically toggled on and off at a frequency; detecting infrared radiation emitted from the test region with a thermal detector; recording and measuring a derivative of the detected infrared radiation to determine a rate of change of emitted thermal radiation with respect to the incident radiation's toggle frequency; correlating the rate of change of emitted thermal radiation to concentration of bound nanoparticles and analyte; and any subcombinations of the above features.

154. A method of calculating and reporting concentration of an analyte, comprising: subtracting a detected thermal radiation signal from a baseline reading to create a result; comparing the result with pre-determined calibrated values stored in memory; correlating an amplitude, maximum, and/or average value of the detected thermal radiation signal to concentration of bound nanoparticles and analyte; and any subcombinations of the above features.

155. A method of calculating and reporting concentration of an analyte, comprising: subtracting a detected thermal radiation signal from a baseline reading to create a result; comparing the result with a similar measurement performed on a calibration region or regions, wherein the calibration region/regions have a known quantity of nanoparticles bound to the surface; correlating an amplitude, maximum, and/or average value of the detected thermal radiation signal to concentration of bound nanoparticles and analyte; and any subcombinations of the above features.

156. A method of fabricating a test region of an assay cartridge, comprising: providing a supporting substrate composed of one or more layers; forming an opening or hole in the supporting substrate at the location of the test region, with the hole cutting through the entire thickness of the supporting substrate; adhering a layer of material of the test region of the nanoparticle assay system of any one of alternatives 126 to 129 and/or another nanoparticle assay system to a surface of the supporting substrate via an adhesive liner including a matching opening or hole; depositing a thin layer of infrared transparent material for attachment of conjugation chemistry; cleaning and surface treatment by plasma and/or chemical means to activate the surface for chemical/bio conjugation; attaching conjugation chemistry to a surface of the test region; selectively placing capture probes only to the surface of the test region which lies directly over the hole or opening in the supporting substrate; and any subcombinations of the above features.

157. A method of measuring a radiation signal of a nanoparticle assay using the test region of the test region of the nanoparticle assay system of any one of alternatives 126 to 129 and/or another nanoparticle assay system, comprising: placing a detector behind the assay cartridge such that a camera images the test region from the backside, or the side opposite to the surface where the assay takes place; measuring radiation emitted through the surface of the test region at the location of the hole or opening in the test region; and any subcombinations of the above features.

158. A method of performing a nanoparticle assay using the nanoparticle assay system of any one of alternatives 112 to 129 and/or another nanoparticle assay system, the method comprising: providing an assay cartridge having one or more test regions and/or another assay cartridge; flowing buffer and/or similar solution to the test region; exposing the test region, before contact with any sample solution, to a source of radiation and measuring a baseline reading, of light emitted through the transparent surface of the test region, with the radiation detector using the method of alternative 157 and/or another method; dispensing the sample, containing analyte/analytes of interest to the test region, wherein the sample is propelled towards the test region by external pressure and/or is manipulated by pipetting; allowing the sample to react with the test region for a duration configured so that present analyte(s) of interest to bind to the test region; washing and/or rinsing the test region with buffer and/or similar solution one or multiple times; exposing the test region to a solution containing nanoparticles conjugated with analyte binding capture probe molecules for a pre-set period of time; washing and/or rinsing the test region with buffer and/or similar solution one time or multiple times; exposing the test region to incident radiation; detecting the scattered radiation emitted through the transparent surface of the test region with the radiation detector using the method of alternative 157 and/or another method; calculating and reporting the concentration of analyte by analyzing the detected thermal radiation signal; and any subcombinations of the above features.

159. The method of alternative 158, where the test region is not washed before exposing the test region to radiation and detecting the radiation signal.

160. The method of alternative 158 or 159, where the test region is not washed before dispensing solution with the nanoparticle complexes toward the test region.

161. The method of any one of alternatives 158 to 160, where the test region is not washed before exposing the test region to radiation and detecting the thermal radiation signal.

162. The method of any one of alternatives 158 to 161, further comprising removing the bulk of the fluid over the test region prior to exposing the test region to incident energy and measuring the thermal response.

163. A method of performing a nanoparticle assay using the nanoparticle assay system of any one of alternatives 112 to 129 and/or another nanoparticle assay system, where the method comprises a method of any one of alternatives 130 to 163 and/or another method, the method comprising, prior to exposing a test region to incident energy and measuring a signal: providing a second set of nanoparticles modified with capture probe molecules to attach to the analyte or the capture probe molecules on the first set of nanoparticles already attached to the surface of the capture region; dispensing a solution containing the second set of modified nanoparticles towards the test region; allowing the solution to react with the first set of nanoparticles already attached the surface of the test region for duration configured so that the second set of nanoparticles bind to the first set of nanoparticles already attached to the surface; washing and/or rinsing the test region with buffer and/or similar solution one or multiple times; and any subcombinations of the above features.

164. A system comprising: a sample potentially containing one or more analytes of interest; an assay cartridge comprising a detection region including at least one electronic sensor chip; an electronic sensor chip comprising a test region containing one or more capture regions and including at least one sensing device; analyte binding capture probe molecules on the surface of the capture region(s) on the sensing device; nanoparticles conjugated with analyte binding capture probe molecules; a source of radiation wherein the radiation excites the nanoparticles to produce a measurable response; and any subcombinations of the above features.

165. The system of alternative 164, where the detection region comprises an array of electronic sensor chips.

166. The system of alternative 164 or 165, where the test region comprises an array of sensing devices.

167. The system of alternative 166, where individual sensing devices in the array are isolated from each other at a substrate level by trenches between the individual sensing devices.

168. The system of alternative 166 or 167, where each of the sensing devices in the array is functionalized with same capture probe molecules.

169. The system of alternative 166 or 167, where each of the sensing devices in the array is functionalized with different capture probe molecules.

170. The system of alternative 166 or 167, where groups of sensing devices in the array are functionalized with capture probe molecules being different between the groups.

171. The system of any one of alternatives 164-170, where the sensing device comprises one or more semiconductor devices, diodes, transistors, resistors, thermistors, resistance thermometer devices, thermocouples, thermopiles, thermostats, bolometers, microbolometers or any combination thereof.

172. The system of any one of alternatives 164-171, where the analyte binding capture probe molecules are coupled to the surface of the at least one sensing device by linker molecules.

173. The system of alternative 172, where the linker molecules comprise one or more chemical molecules or functional silane, where one end terminal end of the linker molecule, or chain of molecules, is bound to the surface of the sensing device and the other end of the linker molecule, or chain of molecules, comprises a functional group capable of binding capture probe molecules.

174. The system of any one of alternatives 164-173, where the nanoparticles comprise one or more layers of gold, silver, carbon, platinum, polymer, plastic, oxide, iron or any combination thereof.

175. The system of any one of alternatives 164-174, where the geometry of the nanoparticles comprises spheres, cylinders, rods, core-shell particles, urchins, stars, plates, cubes, porphysomes or any combination thereof.

176. The system of any one of alternatives 164-175, where analyte binding capture probe molecules are coupled to the surface of nanoparticles by linker molecules.

177. The system of alternative 176, where the linker molecules comprise one or more chemical molecules or functional silane, where one terminal end of the linker molecule, chain of molecules, is bound to the surface of the nanoparticle and the other end of the linker molecule, or chain of molecules, comprises a functional group capable of binding analyte binding capture probe molecules.

178. The nanoparticles assay system of any one of alternatives 164-177, where the capture probe molecules comprise chemical molecules, antibody, enzyme, protein, oligonucleotide, single-stranded DNA, double-stranded DNA, aptamer, DNAzyme, aptazyme, a synthetic molecule capable of binding the analyte or analytes of interest or any combinations thereof.

179. The system of any one of alternatives 164-178, where the analyte or analytes of interest comprise oligonucleotides, proteins, antibodies, chemical molecules or any combinations thereof.

180. The system of any one of alternatives 164-179, where the source of radiation comprises a diode laser, DPSS laser, fiber-coupled laser, light-emitting diode or any combination thereof.

181. A sensor device for detecting nanoparticles in the nanoparticle assay system of any one of alternatives 164-180 and/or another nanoparticle assay system, comprising: an active element which is sensitive to changes in temperature; a layer of thermally insulating material over the active element, the layer of thermally insulating material including one or more patterned openings; a layer of reflective material over the active element, the layer of reflective material including one or more patterned openings; a layer of capping material over the active element, the layer of thermally insulating material, and the layer of reflective material, the layer of capping material comprising the surface of the capture region; a material comprising a thermal mass for heat transfer from the capture region to the active element; and any subcombination of the above features.

182. The sensor device of alternative 181, where the openings in the layers over the active element are aligned such that a continuous opening through the layers exposes the active element or a layer over the active element.

183. The sensor device of alternative 181 or 182, where a thermally conducting material fills the openings.

184. The sensor device of any one of alternatives 181-183, where the capping layer includes openings aligned with the openings in the layers beneath the capping layer.

185. The sensor device of alternative 184, where a thermally conducting material fills the openings in the capping layer.

186. The sensor device of alternative 185, where the capping layer covers the thermal mass.

187. The sensor device of any one of alternatives 181-186, where the thermal mass comprises an oxide, a metal, carbon nanotubes, graphene, graphite or any combination thereof.

188. A sensor device for detecting nanoparticles in the nanoparticle assay system of any one of alternatives 164-180 and/or another nanoparticle assay system, comprising: an active element which is sensitive to infrared radiation; a layer of thermally insulating material over the active element; a layer of reflective material over the active element; a layer of capping over the active element, the layer of thermally insulating material, and the layer of reflective material, the layer of capping material comprising the surface of the capture region; and any subcombination of the above features.

189. The sensor device of any one of alternatives 181-188, where the active element comprises one or more semiconductor devices, diodes, transistors, resistors, thermistors, resistance thermometer devices, thermocouples, thermopiles, thermostats, bolometers, microbolometers or any combination thereof.

190. The sensor device of any one of alternatives 181-188, where the thermally insulating material comprises an oxide, polymer, parylene, aerogel, an air gap or any combination thereof.

191. The sensor device of any one of alternatives 181-188, where the reflective layer comprises a metal, an oxide, a stack of oxides, a dielectric mirror or any combination thereof.

192. The sensor device of any one of alternatives 181-188, where the capping material comprises an oxide, polymer, parylene or any combination thereof.

193. A method of fabricating a bolometer or microbolometer device, the method comprising: forming a reflective layer on a substrate; forming a thermally insulating layer over the substrate; forming a thermistor layer over the thermally insulating layer, where forming the thermistor layer comprises forming at least two electrical contacts; forming openings in the thermally insulating layer; forming electrically conductive vias in the openings; electrically connecting the vias to the electrical contacts of the thermistor layer; forming a layer of thermally insulating material over the thermistor layer; forming one or more materials comprising a reflective layer over the thermistor layer; forming at least one via through the thermally insulating and reflective layers; filling the via with an infrared reflecting or absorbing material; forming a layer of capping material over the layer of thermally insulating material, the one or more materials comprising a reflective layer, and the vias in the thermally insulating and reflective layers, the capping layer comprising the surface of the capture region; and any subcombination of the above features.

### BRIEF DESCRIPTION OF THE DRAWINGS

FIGS. 1A-1F illustrate one embodiments for performing PCR using a test cartridge, wherein a reaction is performed on a substrate containing one or more areas or reaction zones. FIG. 1A depicts a reaction zone on a substrate, and illustrates different configurations for a heat generation layer. FIGS. 1B-1E show comparative non-contact temperature measurements of substrates with pigmented (FIGS. 1B and 1D) or gold (Au; FIG. 1C or 1E) heat generation layers. FIG. 1F shows one embodiment of a reaction zone on a substrate, further including a thermal conduction layer.

FIGS. 2A-2G depict various embodiments for a reaction zone within a test cartridge, in which the reaction region comprises reaction zones having enclosed channels or chambers sandwiched between two substrates. FIGS. 2A-2G show variations of the reaction zones, wherein the substrates have different layers or combinations of layers.

FIGS. 3A-3L illustrate various embodiments incorporating a 3-dimensional line and space pattern into a reaction zone. FIGS. 3A-3B illustrate embodiments of a reaction zone having a well structure to confine the PCR mix solution. FIGS. 3C-3H illustrate embodiments of a reaction zone having a channel and/or chamber structure. FIGS. 3I-3J illustrate embodiments for measuring fluorescence of a PCR mix solution during or after PCR. FIGS. 3K-3L show comparative non-contact temperature measurements of substrates.

FIGS. 4A-4G illustrate embodiments of a track-etched membrane used as a substrate to form a reaction region for PCR. FIGS. 4A-4E show various embodiments of a track-etched membrane. FIGS. 4F-4G show comparative non-contact temperature measurements of a substrate (FIG. 4F) compared to a track-etched membrane (FIG. 4G).

FIGS. 5A-5H illustrate embodiments of a reaction zone in which solid-phase PCR could take place, wherein one or more set of primers may be attached to a solid surface. FIG. 5A depicts one example embodiment of a reaction zone for solid-phase PCR. FIG. 5B-5E depict various embodiments for attachment of a primer to or near a solid surface. FIGS. 5F-5H illustrate embodiments of a reaction zone for solid-phase PCR using a channel/chamber format.

FIGS. 6A-6E illustrate embodiments of an array or arrays of reaction zones in a reaction region of a test cartridge. FIGS. 6A-6C show embodiments or arrays. FIGS. 6D and 6E show cross sectional views of reactions zones from a single row of an array of reaction zones.

FIGS. 7A-7G illustrate embodiments of a vial adapted to allow optical heating of a PCR mix solution. FIG. 7H-7K show gel electrophoresis results of experiments for DNA amplification.

FIGS. 8A-8C illustrate embodiments of a reaction zone with a resistive heater circuit fabricated beneath the reaction zone.

FIGS. 9A-9B illustrate embodiments of a surface of a reaction zone, showing the methods of detection of amplified solid-phase PCR products. FIGS. 9C-9D illustrate embodiments of an array of reaction zones configures for solid-phase PCR detection.

FIGS. 10A-10F illustrate embodiments for building an electrochemical circuit on a reaction zone, configured with an electronically activated heat generation layer (FIGS. 10A-10C) and configured with an optically excited heat generation layer (FIGS. 10D-10F).

FIGS. 11A-11D illustrate an embodiment of a cartridge configured for liquid-phase PCR using an optically excited heat generation layer. FIG. 11A shows a top-view of an embodiment of a cartridge. FIG. 11B shows an exploded view of an embodiment of the cartridge. FIG. 11C shows a side-view of an embodiment of an assembled cartridge. FIG. 11D shows an embodiment of a cartridge inserted into a reader.

FIGS. 12A-12C illustrate an embodiment of a platform for automation of a multi-step diagnostic assay for automated sample processing, mixing, and waste containment. FIG. 12A shows a side view of an embodiment of a circular rotary cartridge. FIG. 12B shows a cross sectional view of an embodiment of a circular rotary cartridge. FIG. 12C shows an embodiment of method of using a circular rotary cartridge for sample assay.

FIGS. 13A-13C illustrate an embodiment of a test region of an assay cartridge.

FIGS. 14A-14E illustrate an embodiment of a method of performing nanoparticle assay using an assay cartridge.

FIGS. 15A-15B illustrate an embodiment of an assay cartridge for increasing a thermal signal for a given concentration of analyte.

FIGS. 16A-16C illustrate an embodiment of a substrate for use in an assay cartridge. As shown in FIGS. 16A and 16B illustrate embodiments of a microfluidic platform, including reaction chambers and enclosed channels, which can be built around and on top of the substrate. FIG. 16C illustrates one embodiment of a thermal detection system using the substrates of FIGS. 16A and 16B.

FIGS. 17A-17B illustrate an embodiment of a substrate fabricated from a thin wafer of semiconductor material or a semi-rigid plastic. Passive sensor strips may be manufactured on the substrates and integrated into a microfluidic assay cartridge or other detection platform.

FIG. 18 illustrates an embodiment of a substrate for use in an assay cartridge. Plasmonic nanoparticles attached to a capture region on the substrate can be detected by measuring light scattering.

FIGS. 19A-19C illustrate embodiments of a substrate for capture of a molecular analyte. FIG. 19A illustrates attachment of nanoparticles to amplify or enhance thermal signal. FIG. 19B illustrates a method of secondary nanoparticle attachment used with an oligonucleotide analyte. FIG. 19C illustrates a method of using secondary nanoparticle attachment in a system where the analyte is, for example, a protein.

FIGS. 20A-20C illustrate an embodiments of the use of a temperature sensitive device or element for nanoparticle detection.

FIG. 21 illustrates an embodiment of a non-contact temperature and/or infrared sensitive element or device that can be used in a nanoparticle assay.

FIGS. 22A-22B illustrate an embodiment of a test region of a sensor chip, showing individual capture regions in an array, with underlying temperature/infrared sensitive devices, as well as sparsely distributed light sensitive devices. As shown in FIG. 22B, light sensitive devices can be built into each capture region.

FIG. 23 illustrates an embodiment of a layout of a sensor chip with integrated processing circuitry.

FIG. 24 illustrates an embodiment of a method for fabricating a microbolometer sensor for compatibility with a nanoparticle assay.

FIG. 25 illustrates one embodiment of a point-of-care nucleic acid amplification and detection system including a test cartridge and a reader device.

FIG. 26 illustrates one embodiment of a test cartridge for nucleic acid amplification reactions.

FIG. 27 illustrates one embodiment of a reader device, configured to receive a test cartridge.

### DETAILED DESCRIPTION

It is to be understood that this disclosure is not limited to the particular embodiments described. It is also to be understood that the terminology used is for the purpose of describing particular embodiments only, and is not intended to be limiting.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, NY 1989). For purposes of the present disclosure, the following terms are defined below.

The articles “a” and “an” are used herein to refer to one or to more than one (for example, to at least one) of the grammatical object of the article. By way of example, “an element” means one element or more than one element.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Any methods disclosed herein need not be performed in the order recited. The methods disclosed herein include certain actions taken by an actor, however, they can also include any third-party instruction of those actions, either expressly or by implication. For example, actions such as “exposing the test region” include “instructing the exposing of the test region.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 3 mm” includes “3 mm.” By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

In PCR nucleic acid amplification, a sample containing target nucleic acids (for example, DNA, RNA) is added to a solution containing other components such as polymerase, primer sequences specific to the target and base nucleotides in a buffer solution. The PCR mixture then undergoes thermal cycling through a range of temperatures to complete the amplification process, typically between 50° C. and 95° C. A PCR cycle involves denaturing of the DNA strand(s) at 95° C., followed by annealing of primers to denatured strands at a lower temperature which depends primarily on primer sequence, typically ˜55° C., followed by extension of nucleic acid strands by polymerase at a temperature somewhere between primer annealing temperature and denaturing temperature, typically ˜72° C. A full PCR process may utilize several cycles of the aforementioned cycle sequence to amplify nucleic acids to a detectable level.

Thermal cycling is typically performed by dedicated bench-top equipment comprising one or more metal heat blocks, which transfer heat to large vials containing PCR mix solution and sample. The large metal heat block represents a significant thermal mass which is uniformly heated to the different temperatures corresponding to different thermal steps in a PCR cycle. Given the large surface area of the heat block, significant thermal energy is required to heat the thermal mass to higher temperatures. Furthermore, due to its high thermal conductivity, heat is also lost to the environment surrounding the heat block during heating. Hence, heating and maintaining a large heat block at the high temperatures used in PCR requires a significant amount of electrical power. Additionally, during the cooling step in a PCR cycle, cooling the heat block rapidly to reduce PCR cycle times requires a powerful dedicated cooling device. Therefore conventional PCR systems are physically large and demand sizable power requirements. Consequently, conventional PCR thermal cyclers are bulky and costly bench-top machines. Furthermore, heat from the heat block must be transferred to PCR mix through a plastic vial containing the PCR mix solution. The insulating plastic of the vial results in inefficient heat transfer to PCR mixture due to the plastic's low thermal conductivity. Because heat must be transferred from the heat block to the plastic vial, any small imperfections in the vial or the surface of the heat block can exacerbate the already inefficient heat transfer. Slow heat transfer to and from the vial can increase the heating and cooling times during PCR cycles, lengthening total test time. To improve heat transfer through the vial, some PCR systems designed for more efficient heat transfer are configured to thinner plastic vials and a heat block with harsh press-fit slots for vial insertion. However, even with these modifications, the overall power and space requirements are unaffected. Furthermore, such systems can be difficult to use due to the forced-insertion of the vials into tiny press-fit slots, often resulting in vials being easily deformed during insertion.

For POC nucleic acid tests (NATs) utilizing PCR, in particular, heating with a conventional heat block or resistive heating element is not an ideal solution. The significant power and cooling requirements of a heat block or heating element as well as the associated bulk and need for perfect contact present a significant obstacle for designing small, portable and efficient POC NAT diagnostic equipment for reliable use in the field.

Finally, end-point or real-time fluorescence detection is typically used to detect amplified DNA products. Real-time or quantitative PCR is popular for in-situ detection of amplified DNA products used in modern PCR systems. There are numerous challenges associated with obtaining reliable results from this type of PCR method, resulting in stringent optimization of each assay. This is exacerbated by the need for multiplexing, wherein multiple DNA targets need to be identified in one sample during a single test. Cost of real-time PCR increases sharply with the number of unique DNA targets to be detected, due to increased complexity and number of reagents. Furthermore, due to the limited number of highly distinguishable fluorescent dyes, requiring different optics, multiplexing is drastically limited in conventional real time PCR.

For widespread application of PCR in POC applications, it is necessary to improve the speed, size, portability, performance, and multiplexing capability of PCR equipment while reducing complexity and cost of assays.

Disclosed herein are several schemes and devices, for amplification and detection of nucleic acids, which can alleviate at least some of the aforementioned limitations of previous approaches and enable new designs for POC NATs.

Lateral flow assays (LFAs) are well suited to some POC applications due to the simplicity and low cost of the devices. However, LFAs generally suffer from insufficient sensitivity and specificity. One type of LFA assay with increased sensitivity is described in PCT/US13/023839 (WO 2013/116333), incorporated in its entirety herein by reference. This LFA employs the plasmonic heating of gold nanoparticles functionalized with antibody as capture molecules. Capture molecules to bind analyte in a sample are physically adsorbed onto a test region on a LFA membrane strip made from porous material. The porous material contains a plurality of interstices and pores. As with most lateral flow strips, the sample is transported to the test region by capillary action. As it flows through the porous strip, it mixes with reagents before reaching the test region. Gold nanoparticles, conjugated with analyte binding molecules, mix with the sample solution that may or may not contain analyte. The gold nanoparticles form nanoparticle/analyte complexes with analyte in the sample solution. The concentration of nanoparticle/analyte complexes formed depends upon the concentration of analyte in the sample solution as well as unbound nanoparticles which also move toward the test region. The capture molecules on the test region bind the nanoparticle/analyte complexes within the test region. The number of nanoparticle/analyte complexes captured and held in the test region depends upon the concentration of analyte that was in the sample solution. Laser energy is then used to heat the gold nanoparticles. The amount of heat generated in the test region is measured by a thermal camera and converted to concentration of analyte in the sample solution.

Referring again to PCT/US13/023839 (WO 2013/116333), unbound nanoparticles could also remain in the test region, which would generate a background signal. Additionally, the laser energy used to heat the gold nanoparticles also heats the porous membrane and the plastic substrate of the LFA test strip, generating a background signal. Due to the randomized 3D structure of the porous membrane of the LFA test strip, it is quite possible that not all bound gold nanoparticles are exposed to the laser energy and therefore do not contribute to the measured signal.

Because LFA techniques typically rely on the sample wicking along the LFA test strip via capillary action, LFA techniques do not lend themselves to the incorporation of wash steps to remove non-specifically adsorbed nanoparticles, causing a non-specific background signal in the measured infrared radiation. The plastic materials used in LFA test strips typically absorb a significant amount of incident laser energy, causing them to heat up and emit infrared radiation, also causing a non-specific background signal. The random structure of the porous membrane of the LFA test strip, to which nanoparticle/analyte complexes bind, causes non-uniform exposure to the incident radiation and therefore variance in the measured infrared radiation. Moreover, the infrared radiation emitted from the nanoparticle/analyte complexes in the bulk of the porous membrane must travel through the membrane as well as the fluid or water present in the sample, which remains in the test region during measurement. Given the high infrared absorption of water, it is possible for the signal to be absorbed before escaping to reach the thermal camera.

Furthermore, LFA techniques do not lend themselves to the incorporation of microfluidic PCR amplification of target DNA that may be required to generate sufficient concentration of target DNA to be detectable by a LFA assay.

In a typical LFA that uses the heating of plasmonic nanoparticles for quantitative analyte detection, the capture molecules of the test region are adsorbed to the test region and within the pores and interstices of the randomized porous membrane material. When a sample solution containing nanoparticle/analyte complexes flows through the test region, some of the nanoparticle/analyte complexes are bound by the capture molecules near the surface of the test region while most are bound within the pores and interstices of the porous membrane. The space within the porous membrane is also occupied by fluid as well as all the other materials present in the sample. Excess nanoparticles not specifically bound to target analyte may also remain in the test region, due to the lack of washing steps to remove them.

When the test region is exposed to radiation to plasmonically heat the nanoparticles, the nanoparticle/analyte complexes bound deep within the porous membrane may be shadowed by nanoparticle/analyte complexes bound above or simply by the membrane material itself. Therefore the amount of heat generated from a given number of nanoparticle/analyte complexes in the test region depends upon how many of the nanoparticle/analyte complexes see the thermal radiation directly. Provided that nanoparticle/analyte complexes bound deep within the porous membrane are exposed to incident radiation, the infrared emitted by the heated nanoparticles would be readily absorbed by water present in the sample. Given water's intrinsic property of high specific heat capacity, the temperature measured by the thermal camera could vary based on the water content in the sample and within the test region. Moreover, in complex samples with hundreds and even thousands of different biological and chemical entities, it is difficult to select a wavelength of radiation which would specifically heat the nanoparticles without being absorbed by at least some of these interfering substances.

With the aforementioned issues as well as self-heating of the membrane and plastic material of the LFA strip, the measured thermal signal or temperature could vary significantly between tests and may lead to inaccurate results or a broad distribution. In order to develop robust assays with washing steps and also enable PCR based DNA/RNA detection applications, it can be advantageous to use a microfluidic platform such as a cartridge where the sample as well as other fluids can be transported to different regions on the cartridge in a controlled manner. However, some of the aforementioned issues are exacerbated in a microfluidic approach due to the excess fluid/water present. Disclosed herein are several schemes, for detection of nanoparticles, which can alleviate at least some of the aforementioned limitations of previous approaches.

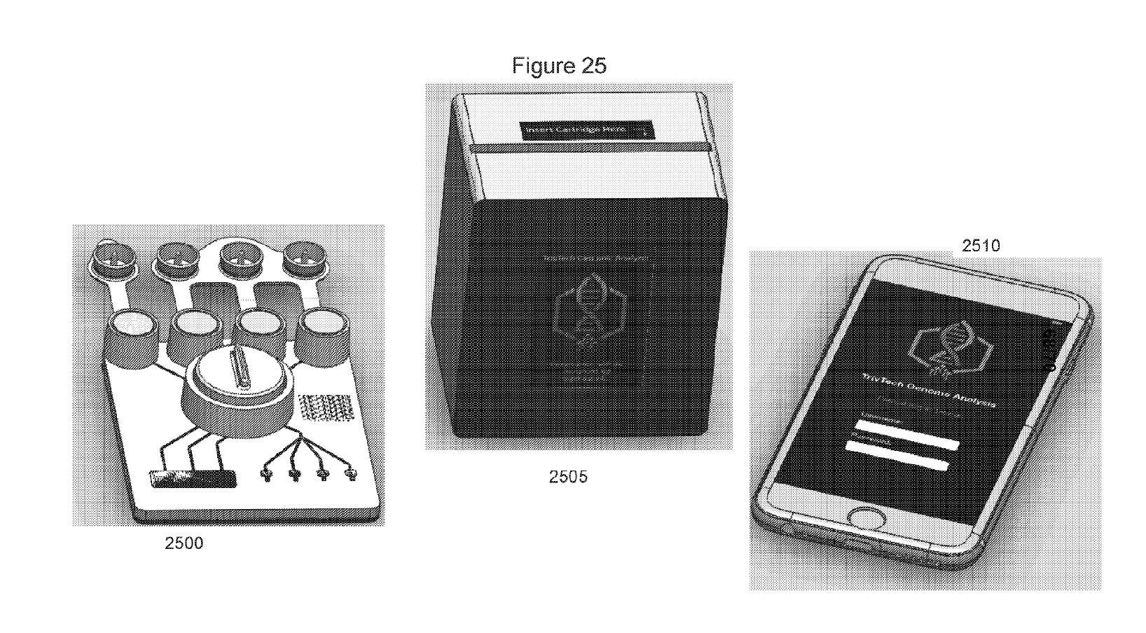

FIG. 25 illustrates one embodiment of a point-of-care nucleic acid amplification and detection system. In some embodiments, the system includes a test cartridge 2500, and an electronic instrument 2505, also referred to as a reader device or electronic reader. In some embodiments, the system further includes a smartphone 2510. In some embodiments, the cartridge 2500 is configured to receive at least one test sample, process at least one test sample to extract and isolate DNA and/or RNA from at least one test sample, and amplify one or more nucleic acid sequences of interest in the DNA and/or RNA of at least one test sample. In some embodiments, the reader device 2505 is designed to interface with the cartridge, automate the various sample processing functions of the cartridge, and detect results of amplification reactions. In some embodiments, the electronic instrument is referred to herein as a reader device. In some embodiments, the electronic instrument or reader device is configured to receive the test cartridge. In some embodiments, the reader device is configured to communicate with smartphone 2510. In some embodiments, the smartphone 2510 is configured with an application, or app, which facilitates bilateral communication with the reader device 2505. In some embodiments, bilateral communication between a smartphone 2510 and a reader device 2505 may include protocol, instruction, status, and/or data transfer. In some embodiments, a smartphone 2510 and its resident interface application, or app, is also configured to capture and process an image of a bar code or QR code on the body of cartridge 2500.

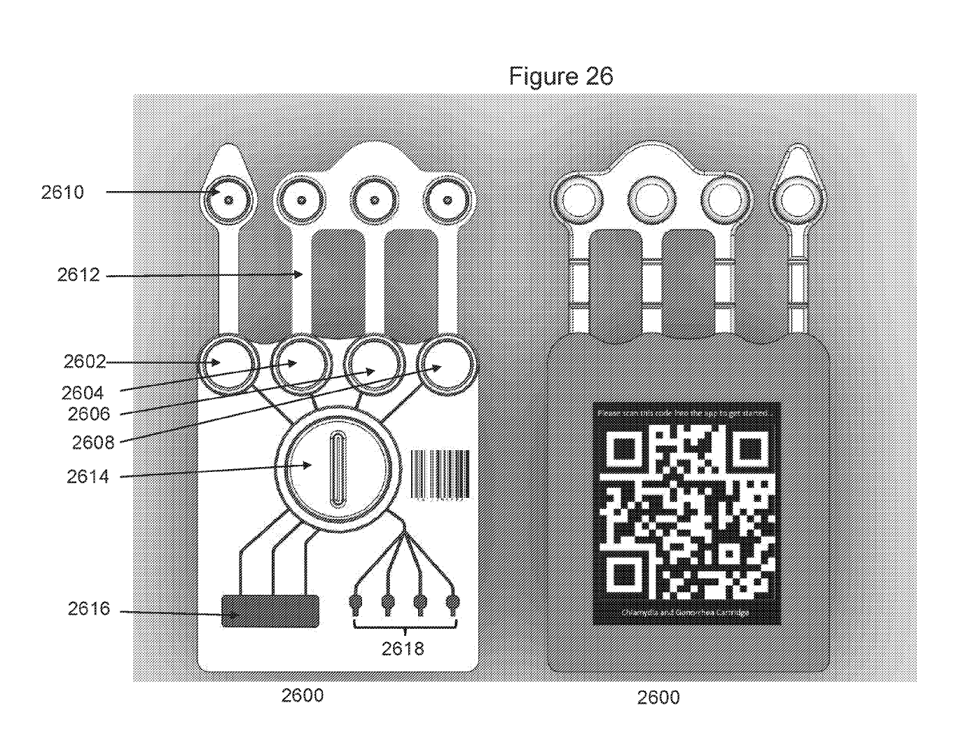

FIG. 26 illustrates one embodiments of a test cartridge 2600, showing one embodiment of a front side of a test cartridge (left) and one embodiment of a backside of the test cartridge (right) 2600. In some embodiments, the backside of the test cartridge includes a bar code or QR code, which may be scanned into a smartphone app by a user to identify the cartridge and to configure a reader device 2505. In some embodiments, a test cartridge is configured to receive one or more test samples. For example, in some embodiments, a test cartridge is configured to receive a bacterial swab and/or a viral swab. In some embodiments, the test cartridge 2600 is configured to receive a single test sample in a sample lysis chamber 202. In some embodiments, chambers 2604 and 2606 contain wash buffers. In some embodiments, chamber 2608 contains elution buffer. In some embodiments, the buffers and solutions in chambers 2602, 2604, 2606, and 2608 may be contained in sealed pouches or blister packs inside the chamber. Additionally, in some embodiments, chamber 2602 may contain dry reagents to promote chemical lysis of target cells in a test sample. In some embodiments, the test cartridge includes capping tabs 2610 and 2612. In some embodiments, capping tabs are configured with puncturing features designed to puncture said pouches or blister packs upon closing the caps, thereby releasing the appropriate fluid in each chamber. In some embodiments, after inputting the sample in sample lysis chamber 2602, a user may seal the chambers using capping tabs 210 and 2612. In some embodiments, the capping tabs may be configured to compress ambient air inside each chamber to pressurize each chamber.

In some embodiments, the test cartridge includes a rotary valve 2614. In some embodiments, the rotary valve contains an internal channel structure to facilitate fluid flow from chambers 2602, 2604, 2606, and 2608 to the other regions of the cartridge in sequence in addition to capture of nucleic acids in an isolation region or chamber within the valve. In some embodiments, rotary valve 2614 is configured to have an internal channel structure filled with materials which capture nucleic acids, specifically silica beads, slurry, gel, fibers or membranes. In some embodiments, these materials promote capture of nucleic acids from test sample from chamber 2602, after which one or more washes may be performed by flowing the wash solutions of chambers 2604 and 2606 through the rotary valve 2614. In some embodiments, the rotary valve physically turns to accommodate sequential switching and/or flowing of fluids in each chamber. In some embodiments, the sample lysate and wash solutions flow through the valve and into waste chamber 2616. In some embodiments, after a final wash the captured nucleic acids inside the isolation region of rotary valve 2614 may be dried by pressurized or ambient air.

In some embodiments, elution buffer from chamber 2608 flows through the rotary valve 2614, resulting in captured nucleic acids within the isolation region of the valve to dissociate from the solid supports and mix into the elution buffer flowing through the valve. In some embodiments, the elute is routed to at least one reaction zone(s) or amplification chamber(s) 2618 by the rotary valve 2614. In some embodiments, the backside of the test cartridge is assembled with a material capable of forming a heat generation layer, for example, the material may be a pigment, dye, doped or undoped semiconductor, compound semiconductor, carbon nanotubes or fullerenes, oxide, polymer, metal, and/or metal alloy. Heat generation layer may be a physically deposited planar layer or may be composed of particles or beads made of any combination of the above materials, for example, gold or silver nanoparticles or polymer beads impregnated with pigment, dye, semiconductor nanoparticles etc. In some embodiments, the backside of the test cartridge is assembled with black pigmented plastic to provide a heat generation layer on the backside of each reaction zone or amplification chamber. In some embodiments, the backside of the cartridge may be assembled with any plastic while leaving a cutout open and unsealed in the reaction zone. In some embodiments, the opening can be sealed with a black pigmented sheet of plastic much thinner (between about 1 micrometer to 3 millimeters in thickness) than the backside of the cartridge, adhered as a strip sealing the backside of the reaction zone; this method may be preferred to reduce heating time and improve heating efficiency. In some embodiments, one or more nucleic acid amplification reactions may take place in any reaction zone or amplification chamber 2618. In some embodiments, each of the reactions zones may contain the same or different sets of dried on-board reagents for identification of one or more target nucleic acid sequences. In some embodiments, the reagents may also include one or my intercalating dyes or molecular beacons which fluoresce incrementally with respect to amplification of specific target sequences, thereby indicating a positive result, as read by the reader device 2505 in the embodiment of FIG. 25.

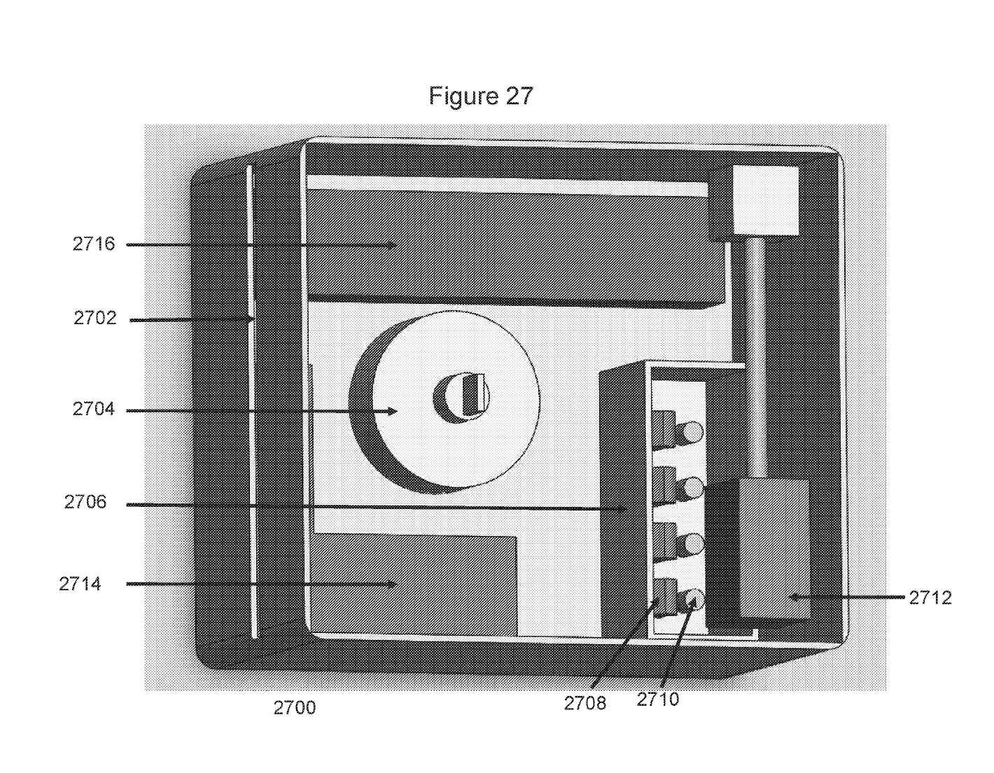

FIG. 27 shows a top-side cut-away image illustrating an embodiment of a reader device 2700, showing an exemplary layout of the components inside a reader device 2700. In some embodiments, the test cartridge is inserted into the instrument through slot 2702. In some embodiments, reader device 2700 houses components and modules designed to interface with the test cartridge so as to actuate certain components of the cartridge, perform and monitor amplification reactions and measure fluorescence resulting from successful amplification reactions. In some embodiments, a keyed feedback controlled stepper motor 2704 is used to actuate the rotary valve on the cartridge. In some embodiments, a heating engine 2706 houses the light sources and infrared detectors used to individually energize the heat generation layers comprising the backside of the reaction zones or amplification chambers. In some embodiments, each light source 2708 is angled and aligned with each infrared detector 2710 such that both their focal points converge onto the same spot on the heat generation layer comprising the backside of each reaction zone, thereby providing optimal temperature tracking and control. In some embodiments, a fluorescence detector module 2712 monitors the fluorescence output of each reaction zone after completion of amplification reactions and/or during the amplification reactions. In some embodiments, a fluorescence detector module 2712 may be motorized to travel between each reaction zone and it may comprise a single or multiple individual emitters and detectors. In some embodiments, the detector(s) may be configured as separate detectors with wavelength selective lenses or a spectrometer with a focusing lens. In some embodiments, a circuit board 2714 includes circuits to control and/or interrogate the stepper motor 2704, heating engine 2706, fluorescence detector module 2712 as well as circuits for wireless communication with the smartphone. In some embodiments, a rechargeable battery 2716 provides power to circuit board 2714 as well as other electrical components in the reader device 2700.

In some embodiments, the infrared sensor detects infrared light. In some embodiments, the infrared light is mid- to far-infrared. In some embodiments, mid- to far-infrared includes light having a wavelength from about 3 μm (micrometers) to about 1,000 μm, or a wavelength that is a value within any range therebetween. In some embodiments, the infrared light has a wavelength that is from about 4 to about 16 μm, or any range therebetween. In some embodiments, the infrared light has a wavelength that is from about 8 to about 14 μm, or any range therebetween.

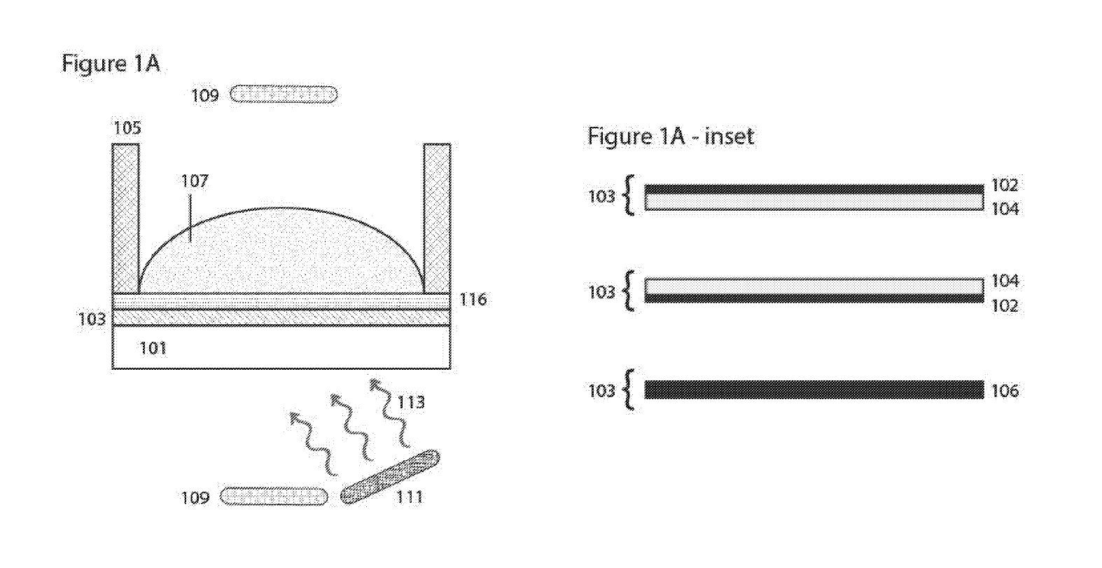

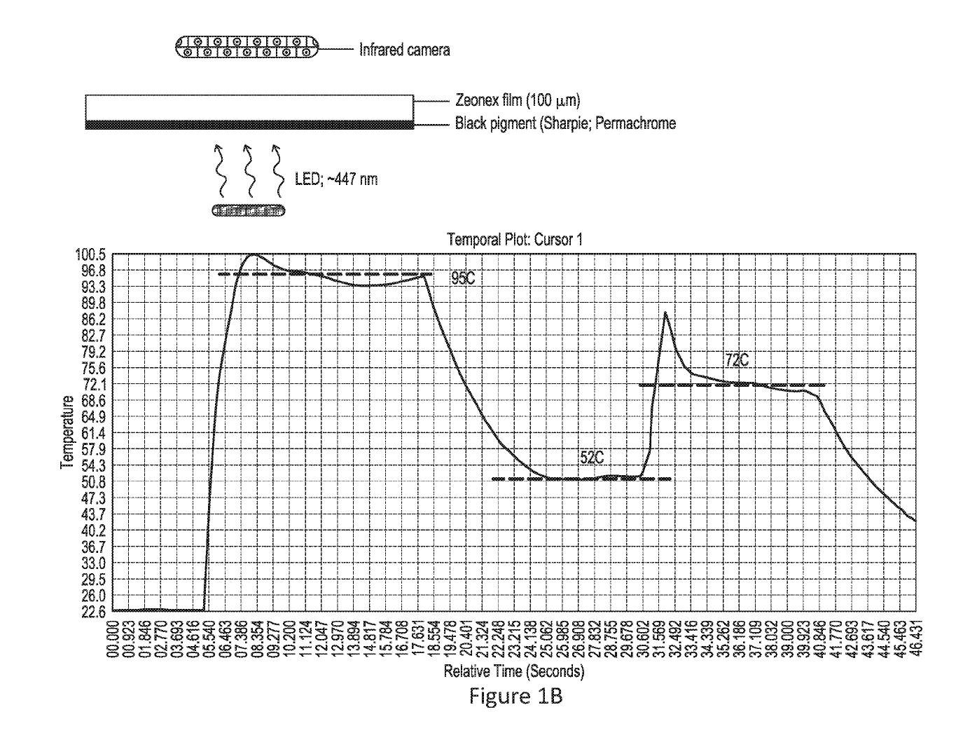

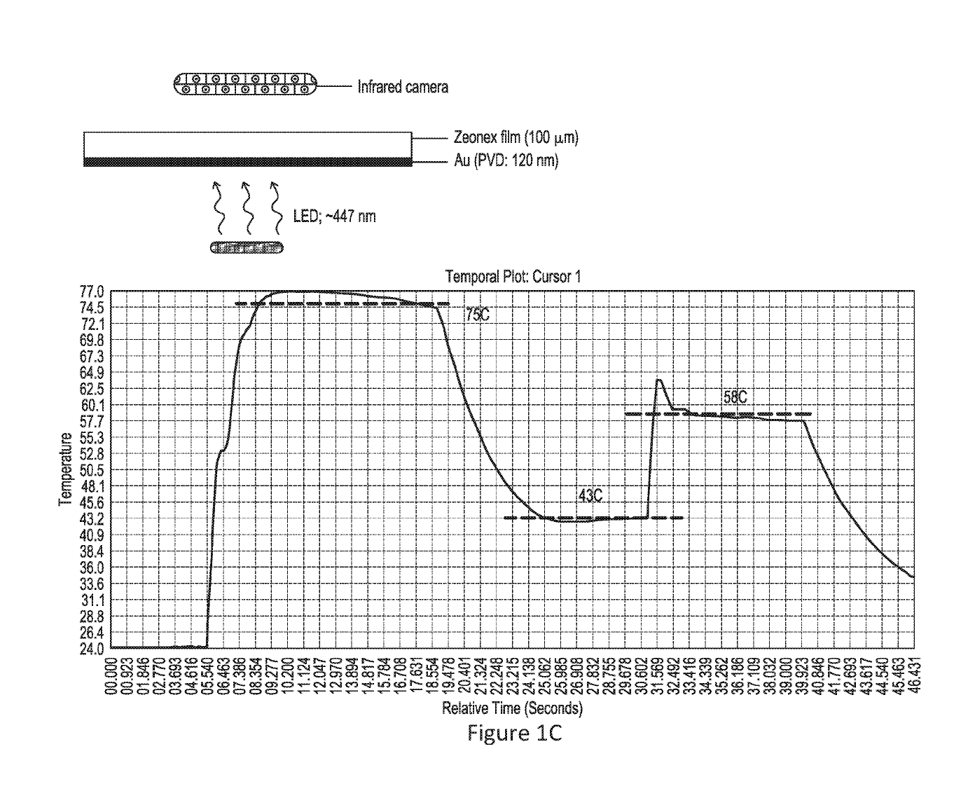

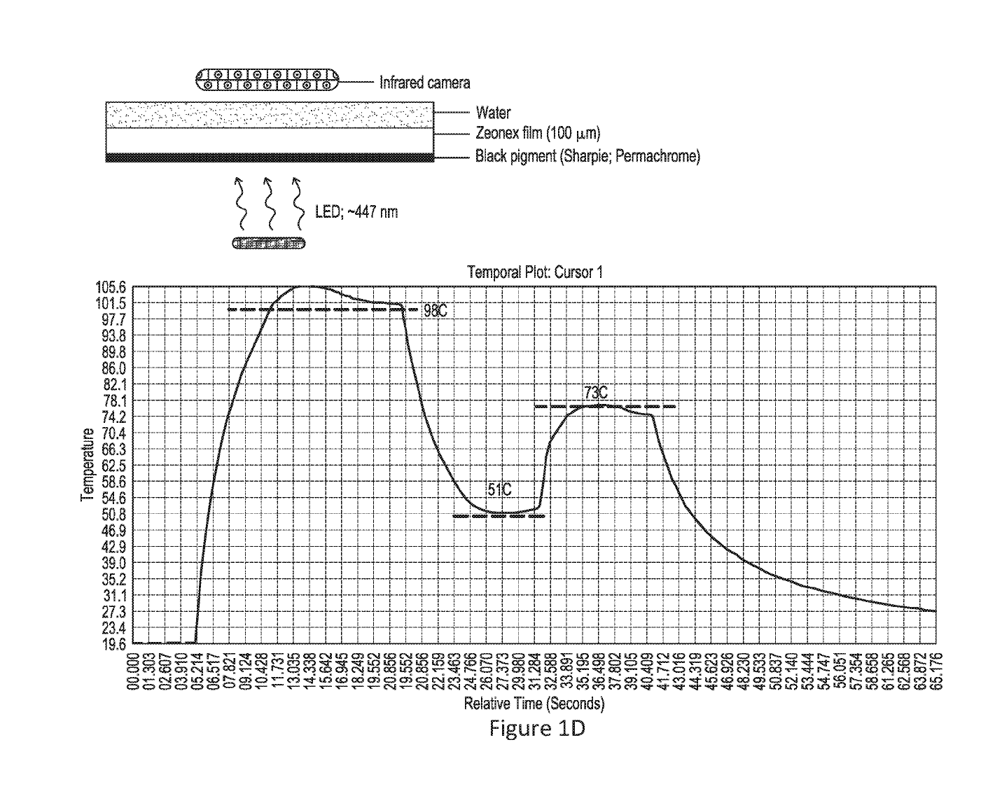

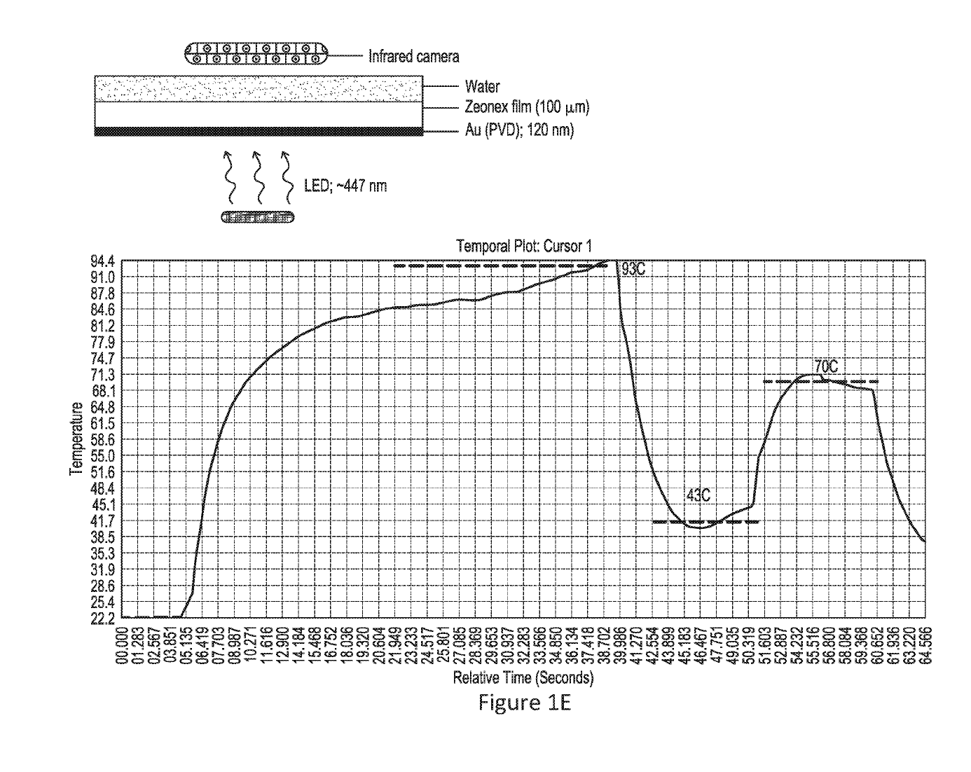

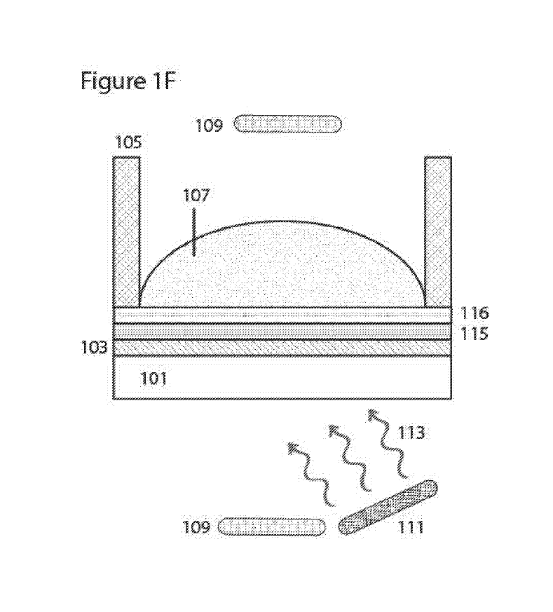

In one embodiment, shown in FIG. 1, an exemplary device for performing PCR on a test cartridge as described herein is provided. In this embodiment, the reaction is performed on a substrate containing one or more areas or reaction zones within which temperature of the liquid sample is cycled during the PCR reaction. The substrate 101 shown in FIG. 1A may constitute one such reaction zone on a larger substrate. The substrate 101 may be plastic, glass, semiconductor or metal; in this embodiment, substrate 101 is a plastic sheet or film. An optically excitable heat generation layer 103, which is heated upon exposure to optical energy, is deposited on top of substrate 101. Heat generation layer may also be deposited on the bottom in conjunction with on the top or only on the bottom of the substrate; for the purpose of this embodiment, heat generation layer 103 is deposited on the topside of substrate 101. Energy source 111 generates light 113 which excites heat generation layer 103. In the case that the heat generation layer is deposited on top of the substrates and it is excited with a light source from below, the substrate should be transparent to allow light to pass through and excite the heat generation layer. The energy source may be a one or more light emitting diodes and/or laser diodes. The light source 111 may also contain focusing and/or collimating optics to focus the light onto the heat generation layer into a spot having high power density. The temperature of the heat generation layer rises upon exposure to the excitation light 113 and is measured by a non-contact thermal sensor 109, placed underneath the substrate 101 in this embodiment. Alternatively, thermal sensor 109 may be placed above the substrate 101 and liquid sample 107, such that the thermal sensor is exposed to but does not directly contact liquid sample 107. By placing the thermal sensor on top, where it is exposed to the liquid sample, the sensor may measure the temperature of the liquid sample more directly because water, present in the liquid sample, is a good absorber and hence, a good emitter of infrared energy. Thermal sensor 109 may be an infrared sensor such as a charge coupled device (CCD), photodiode, thermopile or bolometer device. The temperature of metal or semiconductor surfaces can be difficult to measure with non-contact infrared sensors due to the materials' low emissivity. At higher infrared wavelengths, for example, 8-14 micrometers, measurement error can be significant. To generate accurate readings of the temperature of the heat generation layer, it may be necessary to utilize non-contact sensors optimized for detection between 800 nanometers to 5 micrometers. Therefore, if measuring temperature of the heat generation layer, thermal sensor 109 may be a sensor optimized for detection in the near-infrared spectrum, such as photoconductors made from for example, PbS or PbSe, photodetectors made from for example, Ge, InGaAs or black/porous silicon. Alternatively, thermal sensor 109 can also be integrated into the reaction zone(s) such that it contacts the liquid sample directly. A liquid sample 107 is shown inside well structure 105. The liquid sample 107 used in PCR is typically a mixture of polymerase enzyme, buffer salts, free nucleotides, target DNA, primers and water, known as PCR mix solution. Well structure 105 may be one of many wells on the substrate comprising multiple reaction zones. The well structure(s) may be fabricated with photoresist, polymer, plastic, silicone, polydimethylsiloxane, rubber, dielectric etc. The well structures can be fabricated using molding, photolithography, imprinting, 3D printing or inkjet printing. Alternatively, the well structures be fabricated separately and adhered or bonded to the substrate and/or heat generation layer. The surfaces of the well(s) which are exposed to PCR mix solution 107 may additionally be coated with the same material used in the heat generation layer (or other layers discussed in the embodiment of FIG. 1F), thereby increasing the surface area for conduction of heat to the liquid; any such other layer deposited on the well structures may also be further coated with silane and/or adsorbed protein to prevent interference with the PCR reagents.

Heat generation layer 103 may be a pigment, dye, doped or undoped semiconductor, compound semiconductor, carbon nanotubes or fullerenes, oxide, polymer, metal, and/or metal alloy. Heat generation layer 103 may be a physically deposited planar layer or may be composed of particles or beads made of any combination of the above materials, for example, gold or silver nanoparticles or polymer beads impregnated with pigment, dye, semiconductor nanoparticles etc.

A plasmonic metal, such as gold or silver, is one choice for the heat generation layer, as plasmonic materials are known to selectively absorb certain wavelengths of light which induce surface plasmon resonance in the material. This plasmon induced light absorption can induce heating of the plasmonic material primarily via superheated free or unbound electrons in the metal which conduct heat through the material. However, due to low absorption coefficient, the optical penetration depth in metals can be high, requiring a thick layer for optimal absorption. However, as thickness of the metal is increased, the thermal mass is also increased, thereby exacerbating heat dissipation and requiring higher optical power. Furthermore, metals have very high reflectivity throughout much of the visible light spectrum, limiting optimal absorption to a narrow range of the optical spectrum for optimal plasmon resonance. Typical metals exhibiting resonance assisted absorption, such as gold, are generally high-cost precious metals.

One alternative to using a plasmonic metal for optical heating is to use a polymer or plastic film which intrinsically has high absorption at certain wavelengths. Polyimide film, for example, Kapton®, would be a suitable heat generation layer as it absorbs blue to green wavelengths, resulting in surface heating. Another alternative is a semiconductor material, such as silicon or germanium. A semiconductor material constitutes a more efficient and significantly more economical and versatile heat generation layer. Semiconductors exhibit a band gap and optically excited electrons are propelled beyond the band gap and into the conduction band. Excited electrons undergo a variety of recombination processes which cause electrons to lose large amounts of energy with each recombination event. Energy from these recombining excited electrons is transferred via phonons and thermal vibrations. Phonons transport thermal energy efficiently throughout the lattice, because unlike the electrons in a metal, there is net movement of phonons throughout the lattice of the semiconductor during thermal conduction. Certain semiconductors, such as germanium, exhibit a considerably higher absorption coefficient throughout much of the visible spectrum, resulting in significant absorption of broadband light. Also, due to the high absorption coefficient of germanium, the optical penetration depth in the material is smaller compared to plasmonic metal. For example, germanium's absorption coefficient remains between about 10 to 100 times higher than that of silicon between 400 and 800 nm wavelengths, with penetration depth being as little as about 15 nm for a wavelength of 400 nm. Therefore, a much thinner layer of germanium is can be used to absorb incident light effectively, simplifying, speeding up and reducing the cost of the deposition process. Furthermore, unlike in a plasmonic metal, intense localized heat can be generated in an optically excited semiconductor like germanium, due to its comparatively lower thermal conductivity, which results in less lateral heat dissipation. Additionally, Germanium has a relatively lower specific heat capacity, compared to other common semiconductors like silicon, resulting in quicker increase in temperature for a given input optical energy. Moreover, semiconducting materials, such as germanium, can cost as much as 10 to 100 less than plasmonic precious metals such as gold, making them significantly more economical for use in mass produced test cartridges. Semiconductors also spontaneously develop native oxides on their surface upon exposure to moisture in the air; the native oxide can form a natural passivation layer, preventing interference with PCR reagents and components such as the polymerase. Therefore, it is preferable to use a germanium as the optically excitable heat generation layer, as compared with plasmonic metal such as gold.

Another alternate scheme to make a heat generation layer is to utilize pigments and/or dyes as absorbers of optical energy. A substrate, such as a film of plastic, can be infused or coated with one or more pigments and/or dyes. The pigment particles and/or dye molecules in or on the substrate generate thermal energy upon absorbing incident optical energy and transfer the heat to the substrate and any subsequently coated layers as well as the PCR mix solution. For example, inset 1 of the embodiment shown in FIG. 1A illustrates different configurations for a heat generation layer comprising a plastic film and pigment or dye. In the first embodiment of the inset, heat generation layer 103 is comprised of a plastic film 104 with a coating of pigment and/or dye 102 on the top. The pigment and/or dye may be coated on either side or both sides of the plastic film. In the second embodiment of the inset, heat generation layer 103 is comprised of a plastic film 104 with a coating of pigment and/or dye on the bottom. In the third embodiment of the inset, heat generation layer 103 is comprised of a plastic film 106 which has been pre-infused with pigment and/or dye. Additionally, the heat generation layer 103 can also comprise a layer of pigment or dye molecules coated directly on underlying substrate 101. In the embodiments in the inset of FIG. 1A, the coatings are shown to be black pigment or dye, but can also be other colors. Commercially available dyes or pigments, including inks such as India Ink, Sharpie® ink, Permachrome®, Ultrachrome® etc., may be used as absorbers for generating heat in the heat generation layer. Pigment/dye color can be matched with the excitation wavelength of light source 111 such that the pigment/dye exhibits strong absorption at the wavelength of incident light 113. Alternatively, black pigment/dye, which offers broadband absorption, can also be used and is preferred.

FIGS. 1B through 1E show comparative non-contact temperature measurements of substrates with pigmented vs. gold heat generation layers. The experimental setup used to generate these results is described below. The substrate is Zeonex® plastic film with a thickness of 100 micrometers. The heat generation layer is applied or deposited on the bottom of the Zeonex® film. The pigmented heat generation layer is applied by simply using a black Sharpie® marker to coat the bottom surface of the Zeonex® film; black Sharpie® ink contains Permachrome® pigment. The gold heat generation layer is deposited to a thickness of 120 nm in an electron beam physical vapor deposition system. The heat generation layer is excited by light output from a LED source with peak output at ˜447 nm. An ultra-thin gauge thermocouple is adhered to the heat generation layer on the bottom of the Zeonex® film with transparent tape to monitor the temperature of the heat generation layer. The temperature measured by the thermocouple is used to control the temperature of the heat generation layer by varying the light intensity output by the LED using a closed loop PID control protocol. An infrared thermal camera (FLIR T420) is placed above the Zeonex® film to measure the temperature of the top-side of the Zeonex® film. The graphs show the temperature of the top-side as measured by the infrared thermal camera. The temperature of heat generation layer is cycled between set-points 95° C., 52° C., and 72° C. to simulate a PCR thermal cycle.

FIGS. 1B and 1C compare the performance of the pigmented vs. gold heat generation layers, respectively, as measured in the dry condition. As seen in FIG. 1B, the temperature of the top surface of the Zeonex® film with a pigmented heat generation layer quickly reaches the set-point temperatures (the overshoot can be eliminated by careful tuning of PID parameters). The measured temperature ramp rate during heating is approximately 25° C./sec. However, as seen in FIG. 1C, the temperature of the top surface of the Zeonex® film with a gold heat generation layer does not seem to reach the set-point temperatures, due to the low emissivity of the gold film. FIGS. 1D and 1E compare the performance of pigmented vs. gold heat generation layers, respectively, as measured in the wet condition. A 10 microliter drop of water is placed on the surface of each Zeonex® film such that the infrared thermal camera measures the temperature of the heated water. As seen in FIG. 1D, the temperature of the water atop the Zeonex® film with a pigmented heat generation layer stabilizes very close to the set-point temperatures for all three set-points. The measured temperature ramp rate during heating is approximately 15° C./sec, slower than the dry condition due to the additional thermal mass of the water. However, as seen in FIG. 1E, the temperature of the water atop the Zeonex® film with a gold heat generation layer increases much slower and sometimes does not reach the set-point temperature. Additionally, the electrical power used by the LED light source in exciting the heat generation layer is reduced in the case of the pigmented heat generation layer, due to more efficient light absorption and heating of the pigment. Therefore, as seen in the data in FIGS. 1B-E, a pigmented heat generation layer is preferred for simplicity as well as accurate and rapid ramping of solution temperature with a lower power requirement.

The embodiment in FIG. 1F shows another configuration for a reaction zone. A thermal conduction layer 115 may be deposited on top of the heat generation layer 103. The thermal conduction layer may conduct heat from the heat generation layer to the liquid more efficiently and uniformly. The thermal conduction layer is preferably made from a material having higher thermal conductivity. Thermal conduction layer 115 can be a metal, semiconductor, or metal alloy. An optically reflective material, such as silver or aluminum, may be preferred in that it reflects any light which may penetrate through the heat generation layer back into the heat generation layer. The materials used to make the heat generation or thermal conduction layers could interfere with enzymes and/or primers in the PCR mix solution 107 and inhibit the PCR reaction. Therefore, a thin passivation layer 116 may be deposited on top of heat generation layer 103 or thermal conduction layer 115, such that passivation layer 116 is the topmost layer exposed to the PCR mix solution 107. The thickness of passivation layer is desired to be as thin as possible, preferably between 1 nanometer to 1 micrometer, such that conduction of thermal energy to the liquid sample is not significantly impeded. Another advantage of using a semiconductor as the heat generation is its compatibility with semiconductor oxides such as SiO₂, making deposition of passivation layers with good adhesion straightforward. For example, in the embodiment shown in FIG. 1A, passivation layer 116 could simply be the thin native oxide or a deposited silicon dioxide layer. A further coating of silane molecules and/or physisorbed or chemisorbed proteins, such as bovine serum albumin, on passivation layer 116 may be added to further alleviate any potential PCR inhibition.

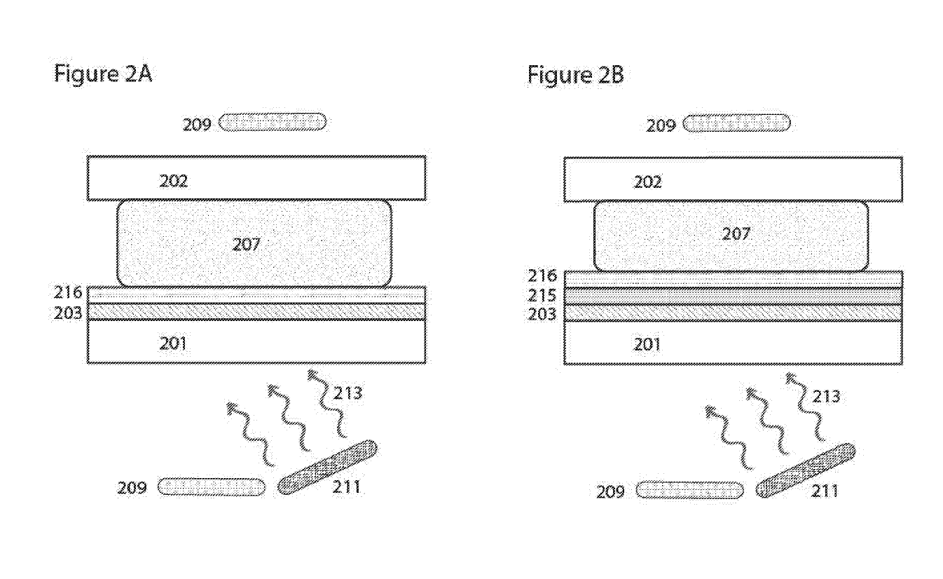

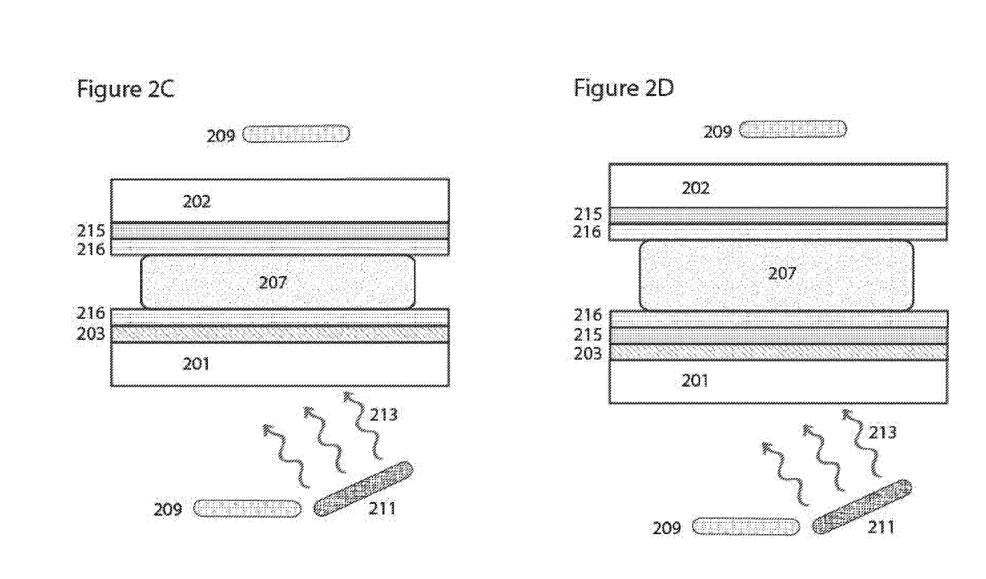

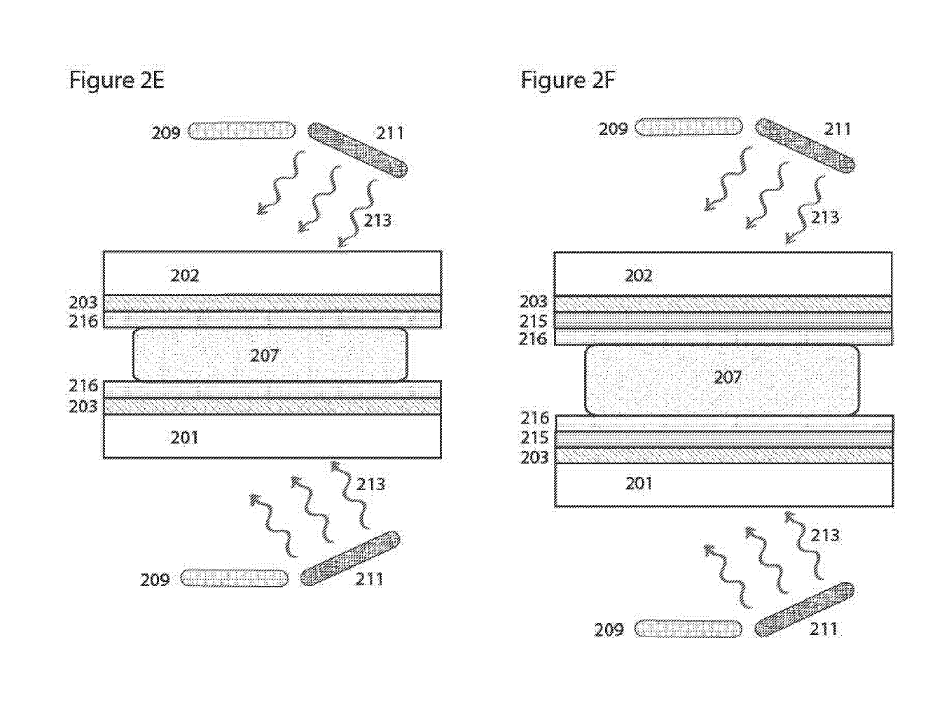

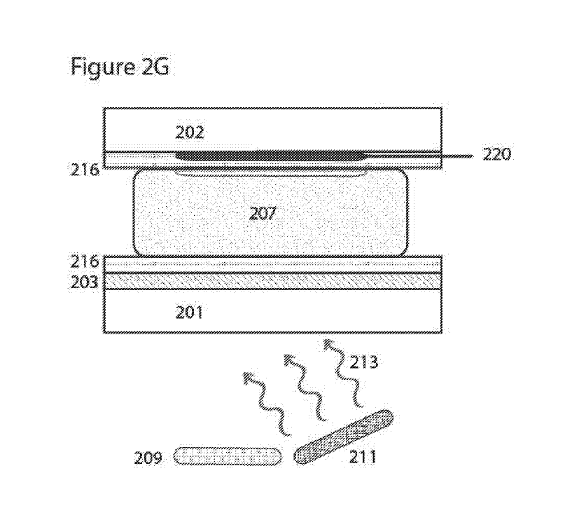

The embodiment shown in FIG. 2, describes another configuration for the reaction zone within a test cartridge, in which the reaction region comprises reaction zones having enclosed channels or chambers sandwiched between two substrates within which temperature of the PCR mix solution is cycled for the PCR reaction. The sample heating and cooling mechanism may be similar to the embodiments shown in FIG. 1. It is to be understood that one or more embodiments described in FIG. 2 may contain components which are similar in their function as the components described in the embodiments of FIG. 1; such components may include the substrate, heat generation, passivation layer, thermal conduction layer, PCR mix solution, energy source, thermal sensor, and light output. Therefore, such components of the various embodiments of FIG. 2 may contain or comprises any of the materials specified for similar components in the description of the embodiments of FIG. 1. For example, heat generation layer 203 could be the same material as heat generation layer 103.

The channel(s) or chamber(s) in the reaction zone(s), in which the PCR reaction is configured to occur, can be assembled in several different ways. The channel(s) or chamber(s) is (are) formed and enclosed by two substrates on the top and bottom of the channel(s) or chamber(s). One advantage of a channel or chamber structure is derived from prevention of evaporation of PCR mix solution during the PCR temperature cycling steps. Another advantage is derived from potentially doubled surface area for heat generation and thermal conduction. PCR mix solution 207 contacts both substrates equally and therefore both substrates may be used to heat and/or cool the liquid for improved performance. In the embodiments in FIGS. 2A-2D, only the bottom substrate is configured for heat generation, while in the embodiments in FIGS. 2E-F, both the top and bottom substrates are configured for heat generation.

In the embodiment in FIG. 2A, the bottom substrate 201 is coated with heat generation layer 203, followed by passivation layer 216. Light source 211 outputs light energy 213 to excite and heat the heat generation layer 203, which transfers heat to the PCR mix solution 207. Substrates 201 and 202 may be made out of a variety of materials including semiconductor, metal, FR-4, polymer, plastic, epoxy, resin, glass, silicone, rubber or any combination thereof. The bottom substrate 201 may be transparent to light energy 213 and therefore may preferably be made of plastic or glass. Top substrate 202 may be made from the same material as bottom substrate 201. Alternatively, top substrate 202 may be made from a material with higher thermal conductivity, which may not necessarily be transparent. Such a configuration may assist in cooling performance, as heat could be transferred out of the PCR mix solution 207 rapidly by a thermally conductive top substrate 202. Utilizing a supplementary cooling device, placed in the reader such that it contacts the top and/or bottom substrates or at least some portion of one or both substrates, may further improve cooling performance. Likewise, a supplementary heating device may also be utilized to improve heating performance. The supplementary heating and/or cooling device(s) in the reader could be a heat sink, electric fan, thermoelectric device such as a Peltier cooler, heat block, resistive heater, printed circuit board heater, and/or flexible circuit or foil heater. Thermal sensor 209 may be placed above top substrate 202 to measure the temperature of the PCR mix solution or to measure the temperature of the heat generation layer 203 and extrapolate temperature of the PCR mix solution 207. Thermal sensor 209 may also be placed below bottom substrate 201 to measure the temperature of the PCR mix solution or to measure the temperature of the heat generation layer 203 and extrapolate temperature of the PCR mix solution 207. Alternatively, the thermal sensor can be built or placed onto the top substrate to directly measure the temperature of the PCR mix solution by contacting the PCR mix solution inside the reaction channel or chamber. The embodiment in FIG. 2G shows thermal sensor 220 fabricated directly on the surface of the top substrate 202. Thermal sensor 220 is then coated with a layer of passivation material forming the passivation layer 216 on the top substrate 202. Thermal sensor 220 is shown as one example of using a contact temperature sensor, such as a thermocouple or thermistor, which can be simply fabricated on a plastic substrate for integration into the reaction channel or chamber. Thermal sensor 220 can simply be fabricated as a thin spiral or serpentine track of thermistor material with electrodes on either terminal of the track for electrical biasing and measurement. The low thermal mass of thermal sensor 220 fabricated on the top substrate 202 in such a manner would enable direct, accurate and rapid temperature measurements of the PCR mix solution.

In the embodiment in FIG. 2B, thermal conduction layer 215 is placed on top of heat generation layer 203. The passivation layer 216 is then placed on top of the thermal conduction layer 215. In the embodiment in FIG. 2C, the bottom substrate 201 contains the heat generation layer 203 and passivation layer 216, similar to the embodiment in FIG. 2A. The top substrate 202 contains a thermal conduction layer 215 coated with the passivation layer 216. This configuration allows the top substrate 202 to be made from a conventional material, like plastic or glass, while still having superior thermal conduction properties for better cooling performance, due to thermal conduction layer 215. In the embodiment in FIG. 2D, a thermal conduction layer 215 is added between heat generation layer 203 and passivation layer 216 on the bottom substrate 201. The top substrate 202 contains a thermal conduction layer 215 coated with the passivation layer 216. In the embodiments in FIGS. 2E and 2F, both the top and bottom substrates are configured with heat generation layers for heat generation. In the embodiment in FIG. 2E, both the bottom and top substrates 201 and 202, are coated with heat generation layers 203, followed by passivation layers 216. Two light sources 211 are used to excite the heat generation layers on both substrates with light energy 213. In the embodiment in FIG. 2F, a thermal conduction layer is sandwiched between heat generation layers 203 and passivation layers 216 on both bottom and top substrates 201 and 202.

Increasing surface area of surfaces which contact the PCR mix solution can improve heating and cooling efficiency and speed. An increase in surface area of the heat generation layer would allow for quicker heating due to the additional surface area of heat generation material to capture light. A larger surface area of the heat generation or thermal conduction layers would also enable rapid conduction of thermal energy to the PCR mix solution. To increase surface area, three dimensional features may be fabricated on the surface of the bottom and/or top substrates such that all subsequent layers coated onto the substrates would conform to the 3D structures of the substrates. One method to generate a 3D structured substrate is to form 3D features directly from or on the surface of the substrate using molding methods. Alternatively, 3D features can be fabricated and/or deposited the substrate using photolithography, screen printing, or inkjet printing techniques to pattern photoresist (for example, SU-8), polymer, spin-on-glass, or other transparent material onto the surface of the substrate. The 3D features on the substrate can take the form of a wide variety of 3D structures in distributed or uniform arrays. Such 3D features may include but are not limited to pillars, droplets, spheres, lines, line and space gratings, sawtooth etc. Additionally, a repeating 3D pattern, for example, a grating, specifically designed specifically for the wavelength of light used may result in enhanced absorption of light by the resulting 3D-structured heat generation layer.

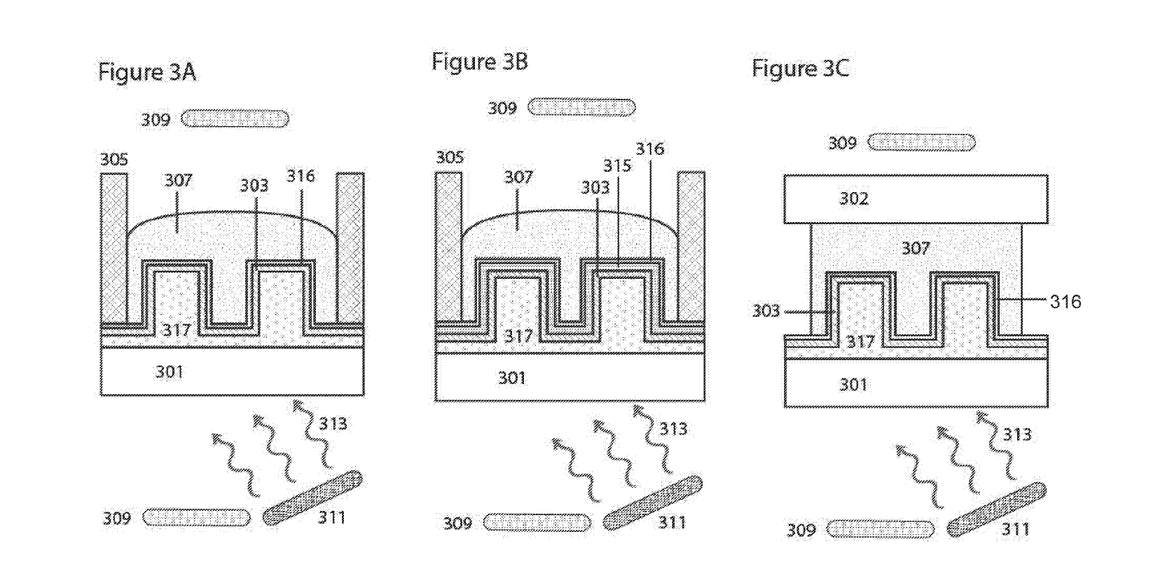

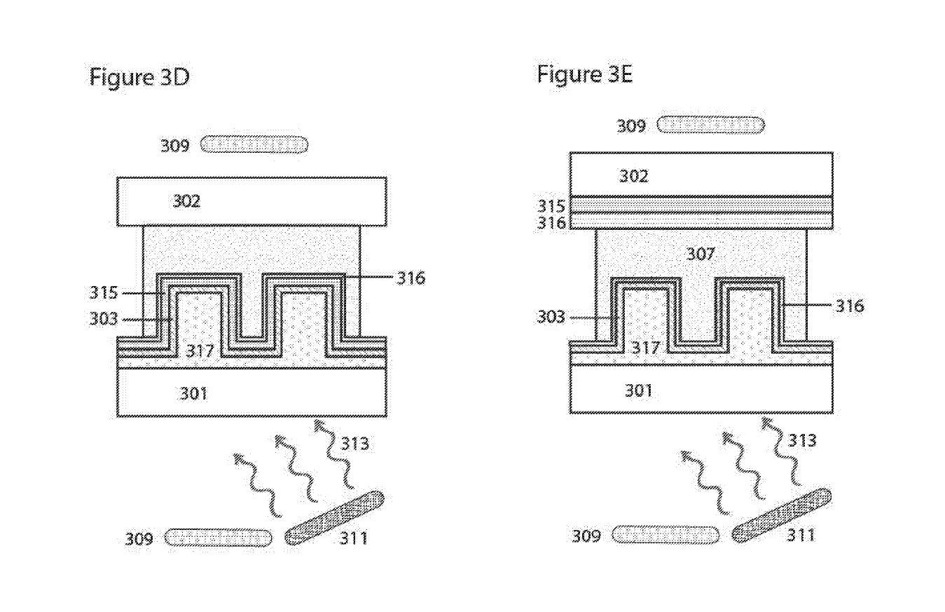

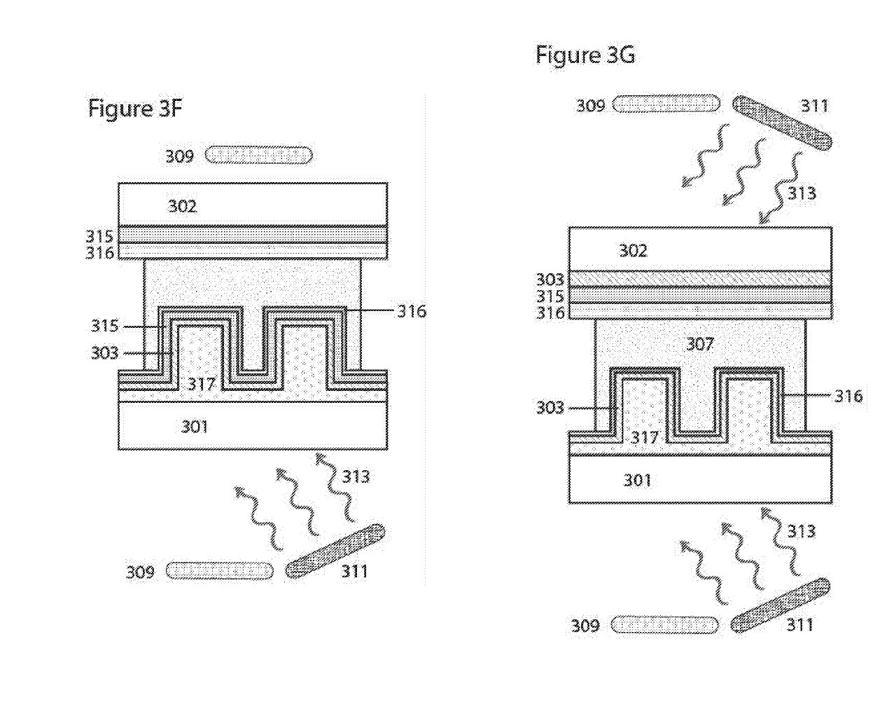

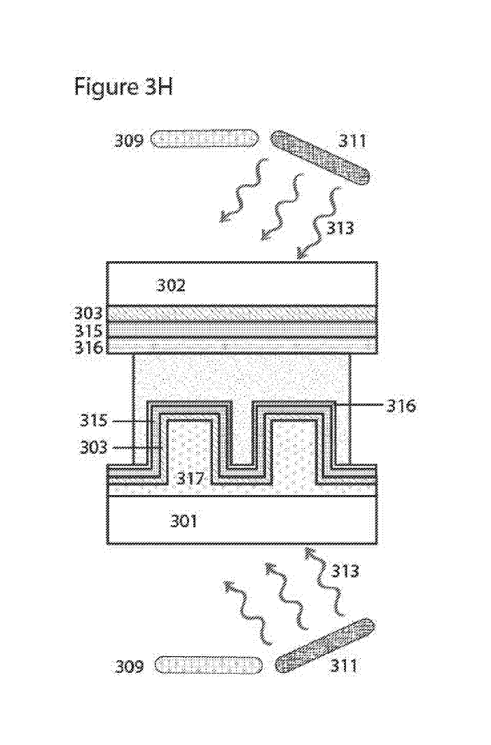

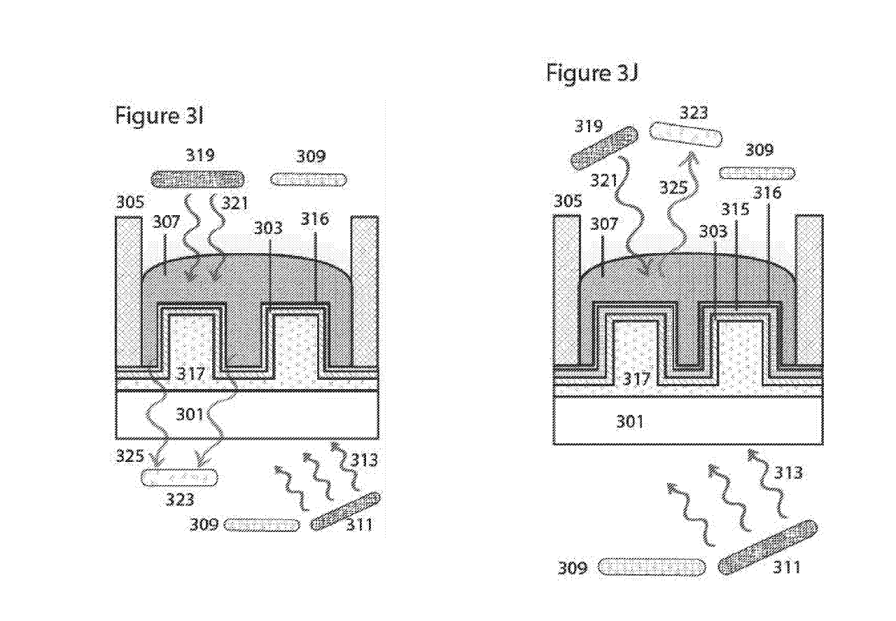

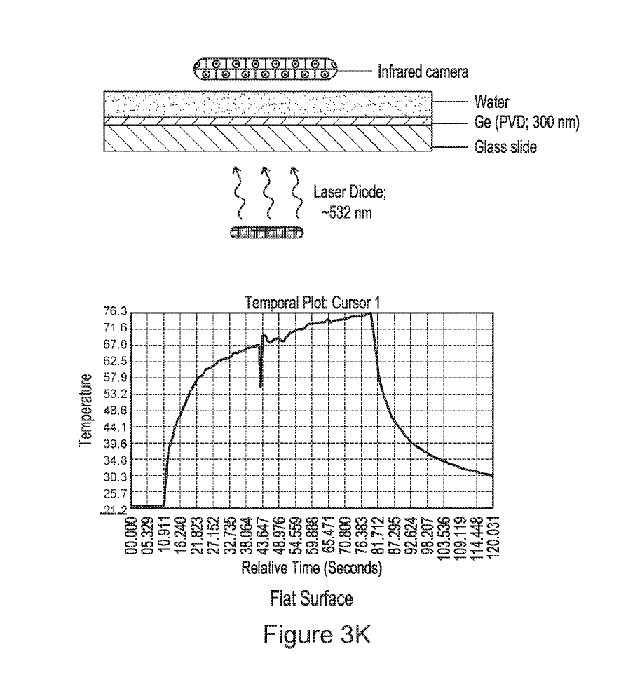

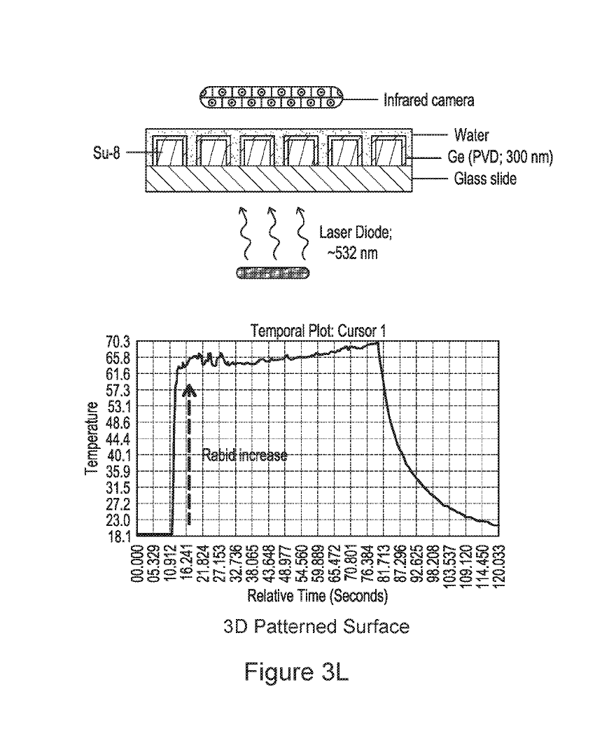

The embodiments in FIGS. 3A and 3B illustrate the incorporation of a 3D line and space pattern into a reaction zone having a well structure to confine the PCR mix solution, similar to the embodiments in FIG. 1. Light source 311 outputs light energy 313 to excite the heat generation layer 303. Thermal sensor 309 measures the temperature of the PCR mix solution or the surface of the heat generation layer. The surface of substrate 301 contains a 3D pattern layer 317, shown as a line and space grating in this embodiment. Heat generation layer 303 is deposited on top of the 3D pattern layer 317 such that it conforms to the shape of the underlying 3D pattern layer 317. A passivation layer 316 is deposited on top of the heat generation layer 303 such that it conforms to the shape of the underlying layers. In the embodiment in FIG. 3B is slightly different from the embodiment in FIG. 3A in that a thermal conduction layer 315 is first coated on top of the heat generation layer 303, followed by passivation layer 316.

The embodiments in FIGS. 3C-H illustrate different configurations to incorporate the 3D pattern layer into reaction zone(s) which have a channel and/or chamber structure. In the embodiment in FIG. 3C, substrate 301 has a 3D pattern layer 317 which is coated with heat generation layer 303, followed by passivation layer 316. The top is capped with substrate 302, forming a channel or chamber. In the embodiment in FIG. 3D, substrate 301 has a 3D pattern layer 317 which is coated with heat generation layer, thermal conduction layer 315 and passivation layer 316. In the embodiments in FIGS. 3E and 3F, the top substrate is coated with an unpatterned thermal conduction layer 315 and passivation layer 316, while the bottom substrate has a 3D structure with layers similar to embodiments of FIGS. 3C and 3D. The embodiments in FIGS. 3G and 3H illustrate a configuration in which a heat generation layer is also deposited on the top substrate, followed by thermal conduction and passivation layers. In this configuration, light source 311 and thermal sensor 309 may also be placed above the top substrate to allow the top substrate to generate heat. The bottom substrate of the embodiment in FIG. 3G contains the 3D pattern layer 317, followed by heat generation layer 303 and passivation layer 316. The bottom substrate of the embodiment in FIG. 3H contains the 3D pattern layer 317, followed by heat generation layer 303, thermal conduction layer 315 and passivation layer 316. It is important to note that a 3D pattern layer could also be fabricated on the top substrate in these embodiments, prior to its assembly into the cartridge (not shown). The 3D pattern layer on the top substrate may or may not be the same as that of the bottom substrate. The resulting dual 3D configuration, in a reaction zone having 3D pattern layers on both the top and bottom substrates, would accelerate heating and/or cooling of the PCR mix solution due to the additional surface area, thereby significantly improving PCR performance. All embodiments of FIG. 3 illustrate that a channel or chamber structure, in which top and bottom substrates enclose the reaction zone(s), can enhance performance of PCR significantly by alleviating evaporation issues and increasing contact area. The unique and versatile utility of the top substrate, wherein it can be used for heat generation and/or an additional thermal conduction for heat transfer, improves overall heating and/or cooling performance. Furthermore, a channel or chamber configuration is readily adaptable to microfluidic or lab-on-chip cartridges.

The embodiments in FIGS. 31 and 3J demonstrate possible configurations to measure the fluorescence of the PCR mix solution during or after PCR. In both FIGS. 31 and 3J, an excitation source 319 generates and exposes the PCR mix solution 307 to light 321 of a wavelength specific to the fluorescent molecule(s) used in the solution to indicate successful amplification. Light sensor 323 detects the emitted fluorescence 325 from the PCR mix solution upon excitation by excitation source 319. The configurations shown in FIGS. 31 and 3J use a well format for the purpose of illustrating the concept; chamber or channel formats illustrated in FIGS. 3C-H can also be adapted for detection in a straightforward manner. The fluorescence output can be monitored during the entire PCR process in a real-time format or simply after the last amplification cycle, in an end-point format.

FIGS. 3K and 3L show comparative non-contact temperature measurements of substrates with a germanium heat generation layer deposited on a flat surface vs. a 3D patterned surface. The experimental setup used to generate these results is described below. The substrate is a glass slide. The germanium heat generation layer is deposited to a thickness of 300 nm in an electron beam physical vapor deposition system. In the case of FIG. 3K, the 300 nm germanium is deposited directly on the flat surface of the glass slide. In the case of FIG. 3L, a 3D line and space pattern is first fabricated on the glass slide using Su-8 photoresist. The Su-8 line and space pattern is 150 micrometers tall, 10 micrometers wide with a spacing of 10 micrometers in between each line. A 300 nm thick layer of germanium is then deposited on the Su-8 patterns. The heat generation layer is excited by light output from a DPSS laser module with peak output at ˜532 nm. The intensity of the light output is maintained at the same level for both samples for the duration of the measurement in both Figures. A drop of water is placed on top of the heat generation layer prior to heating. An infrared thermal camera (FLIR T420) is placed above the glass slide to measure the temperature of the water. As seen in FIG. 3K, in the case of a heat generation layer deposited on a flat surface, the temperature of the water ramps to about 63° C. over the course of about 20 seconds, after which it continues a slower ramp to about 76° C. However, as seen in FIG. 3L, in the case of the 3D patterned surface, the temperature ramps to about 63° C. very quickly over the course of only about 2 seconds due to the increased surface area of the heat generation layer in contact with the water; this is an order of magnitude faster than the sample having a flat surface, used in FIG. 3K. Furthermore, the cooling performance is improved as well, because the sample with the 3D patterned heat generation layer cools down to room temperature (˜20° C.), whereas the sample with the flat heat generation layer cools to only about 30° C. in the same time. This clearly demonstrates the advantage of using a 3D patterned heat generation layer to increase heating and cooling performance during PCR thermal cycling.

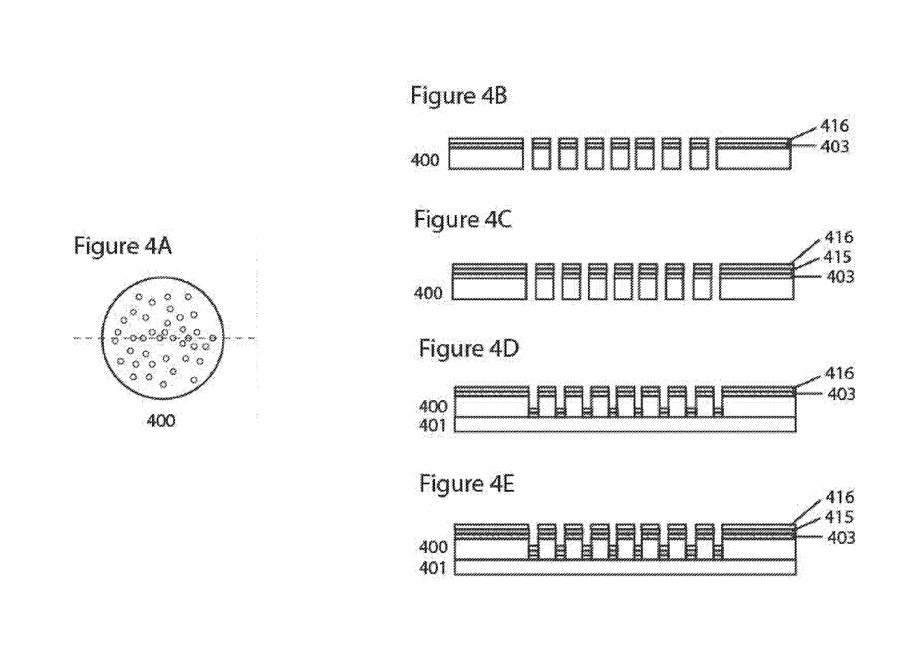

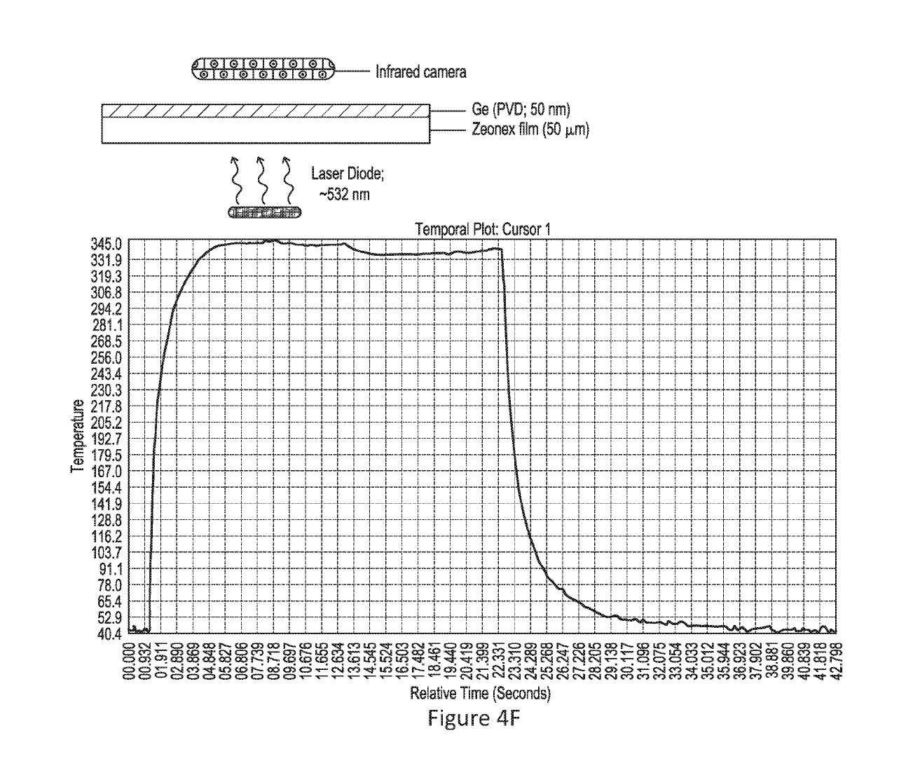

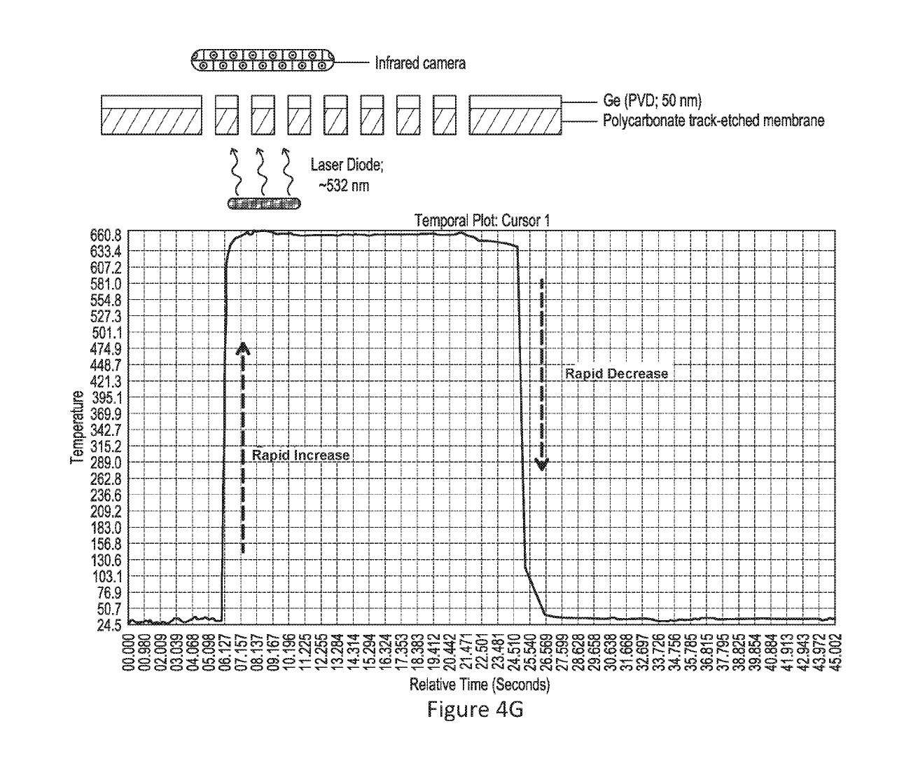

The embodiment in FIG. 4 shows a plastic track-etched membrane 400 which can be used as a substrate onto which subsequent layers such as the heat generation, thermal conduction, and passivation layers can be deposited to form a reaction region for PCR. Due to its small through-holes, which function as a 3D pattern, a track-etched membrane substrate can significantly increase the surface area in contact with PCR mix solution. The embodiments in FIGS. 4B-E show different cross sections of the track-etched membrane 400 to illustrate different configurations of the deposited layers on the track-etched membrane. In the embodiment in FIG. 4B, heat generation layer 403 and passivation layer 416 are deposited onto the surface of the track-etched membrane 400. In the embodiment in FIG. 4C, heat generation layer 403 is deposited on track-etched membrane 400, followed by thermal conduction layer 415 and passivation layer 416. It is also possible to deposit any combination of these layers on both the bottom and top sides of the track-etched membrane 400. The embodiments in FIGS. 4D and 4E illustrate a configuration in which track-etched membrane 400 may be supported by another underlying substrate 401 for structural support. In the embodiment in FIG. 4D, heat generation layer 403 and passivation layer 416 are deposited on track-etched membrane 400 which is bonded or adhered to supporting substrate 401. In the embodiment in FIG. 4E, heat generation layer 403, thermal conduction layer 415 and passivation layer 416 are deposited on the track-etched membrane. In both configurations shown in the embodiments in FIGS. 4D and 4E, the deposited layers may also be deposited in the inner surfaces of or sidewalls of the holes in the track-etched membrane as well as on the exposed surfaces of supporting substrate 401 during the physical deposition process. An angled deposition process may be used to prevent deposition into the holes and limit it to near or on the surface of the track-etched membrane. However, deposition of the heat generation and subsequent layers into the holes and onto the exposed portions of the underlying support substrate could actually lead to more uniform heating, as the solution in the holes would be heated from multiple surfaces. In lieu of depositing a heat generation layer on a track-etched membrane, a black track-etched, typically used in fluorescence applications, could be used as the starting substrate. Black track-etched membranes are typically made directly from pre-dyed or pre-pigmented plastic.

FIGS. 4F and 4G show comparative non-contact temperature measurements of substrates with a germanium heat generation layer deposited on a flat surface vs. a track-etched membrane. The experimental setup used to generate these results is described below. The germanium heat generation layer is deposited to a thickness of 50 nm in an electron beam physical vapor deposition system. In the case of FIG. 4F, the 50 nm germanium is deposited on the flat surface of the glass slide. In the case of FIG. 4G, the 50 nm germanium is deposited on a polycarbonate track-etched membrane having average pore size of about 1 micrometer. The heat generation layer is excited by light output from a DPSS laser module with peak output at ˜532 nm. The intensity of the light output is maintained at the same level for both samples for the duration of the measurement in both Figures. An infrared thermal camera (FLIR T420) is placed above the substrates to measure the temperature of the heat generation layer in dry conditions. As seen in FIG. 4F, in the case of a heat generation layer deposited on a flat surface, the temperature of the heat generation layer ramps to a steady state value in about six seconds. Cooling is slow as well; the temperature settles back down to the low value gradually over the course of at least 15-16 seconds. However, as seen in FIG. 4G, in the case of the track-etched membrane, the temperature ramps to a steady state value very quickly, taking only about one second. Furthermore, using a track-etched membrane improves the cooling performance as well; the temperature settles back down to the low value rapidly in only about two seconds. The rapid heating and cooling performance on the track-etched membrane is attributed to the large increase in surface area of the heat generation layer as it is coated inside the tiny pores of the track-etched membrane. This further demonstrates the advantage of using a 3D patterned heat generation layer to increase heating and cooling performance during PCR thermal cycling. The temperature measurements shown in FIGS. 4F and 4G show relative temperature values, rather than absolute temperature values. The important note from these comparative figures is that the rate of change in temperature is slower in FIG. 4F than in FIG. 4G, meaning that the increased surface area in the setup of FIG. 4G helps speed up heat generation and cooling. Furthermore, the relative values can be compared between FIGS. 4F and 4G, such that the temperature in FIG. 4G is higher, showing a more efficient heat generation layer.

There are various methods to perform PCR amplification reactions and subsequent or simultaneous detection of amplified products. For example, typical liquid-phase PCR could be performed using a two-step or a three-step cycling process. Liquid-phase PCR could also be performed with an isothermal PCR process, without temperature cycling. Detection of amplified products may be achieved by real-time detection of fluorescent molecules or probe primers in real-time during the PCR reaction or by end-point detection of the molecules after conclusion of the PCR experiment. The PCR temperature cycling systems, cartridges and devices disclosed in the previous embodiments may be adapted such that any available method of performing liquid-phase PCR could be used.

In one embodiment, for example, a method for basic liquid-phase PCR reaction using a standard three-step process is described herein. The embodiments of FIG. 1 are used to aid in describing this method below but it is to be understood that this method can be adapted to use of any of the configurations for reaction zones in the embodiments of this disclosure. PCR mix solution 107, which may include target DNA and PCR master mix, is first dispensed into the sample confinement layer 105. The temperature of the liquid sample is then directly measured with thermal sensor 109 facing the PCR mix solution or extrapolated if thermal sensor 109 is facing the heat generation layer. If the thermal sensor is placed above the substrate, such that it captures infrared emitted from the PCR mix solution, then it directly measures the temperature of the liquid. If the thermal sensor is a contact-based sensor and is placed within the sample well(s) of the confinement layer, in direct contact with the PCR mix solution, then it also directly measures the temperature of the liquid by contact. If the thermal sensor is placed below the substrate, such that it captures the infrared emitted from the bottom surface of the heat generation layer, then the temperature of the bulk of the liquid sample is indirectly extrapolated or calculated based on the light output power, light exposure time, baseline/starting temperature, and temperature of the heated heat generation layer. Next, heat generation layer 103 is exposed to light 113 from energy or light source 111 until the temperature reaches at least 95° C., as measured by thermal sensor 109, to allow preliminary denaturing of the DNA template. The energy or light source 111 may be powered on at a steady-state or varying power level; alternatively, the power level may be gradually ramped from zero to a higher value until the desired temperature is reached. During the entire PCR experiment, the temperature of the heat generation layer is maintained at or close to the desired ideal value by tuning the light output 113 of energy or light source 111 based on measurements from thermal sensor 109 using a proportional, proportional-integral, or proportional-integral-derivative (PID) controller circuit or software. Thermal sensor 109 and energy or light source 111, controlled by logic circuitry and/or software, form a closed loop temperature control system for active control of the temperature of PCR mix solution 107. Heating of PCR mix solution to a specific temperature is provided by tuning light output 113, with the aid of the closed loop temperature control system, while cooling is facilitated by the liquid losing thermal energy to the environment and/or the thermal mass of the heat generation 103 and/or thermal conduction layer 115. After the initial denaturing step, the temperature is reduced to an appropriate level to allow for annealing of primers to the DNA template or amplicons. Next, the temperature is increased to a level which allows optimal polymerase activity for primer extension, depending on the type of polymerase used. Next, the temperature is increased to a level appropriate for denaturing of extended DNA (˜95° C.). The PCR mix solution's temperature is then repeatedly cycled between denaturing, primer annealing, and primer extension temperatures in a controlled manner facilitating multiple cycles of PCR. A final denaturing cycle may be performed after all cycles of PCR are completed. Finally, the temperature is reduced to a low value (for example, room temperature, ˜25° C.) to allow qualitative or quantitative measurement of fluorescence from fluorescent molecules or probes, which may be present in the PCR mix solution 107; the molecules will fluoresce if target DNA has been amplified, indicating a successful PCR reaction.

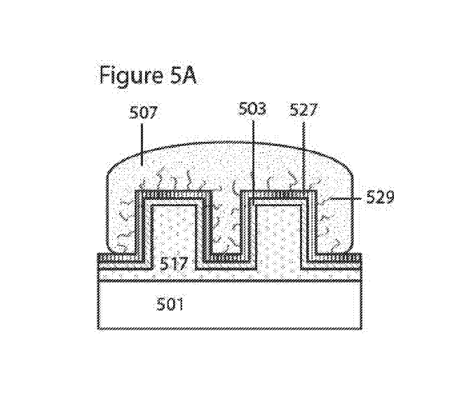

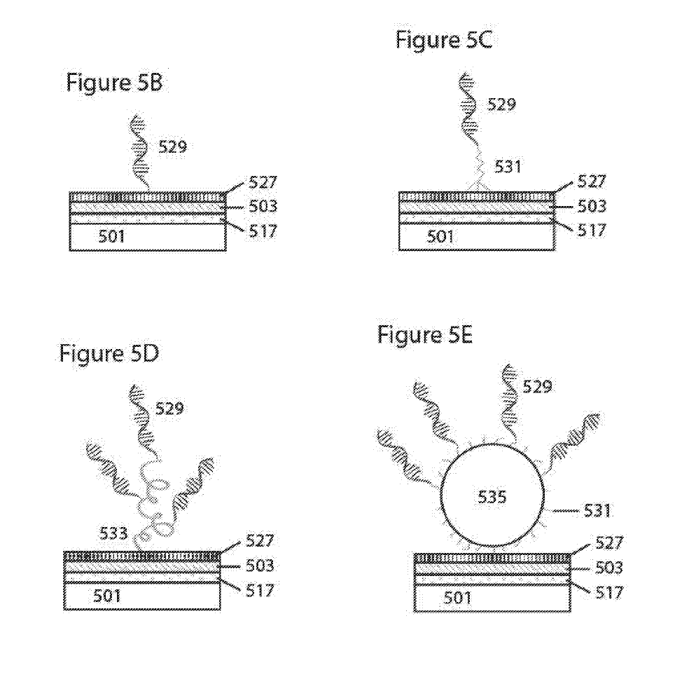

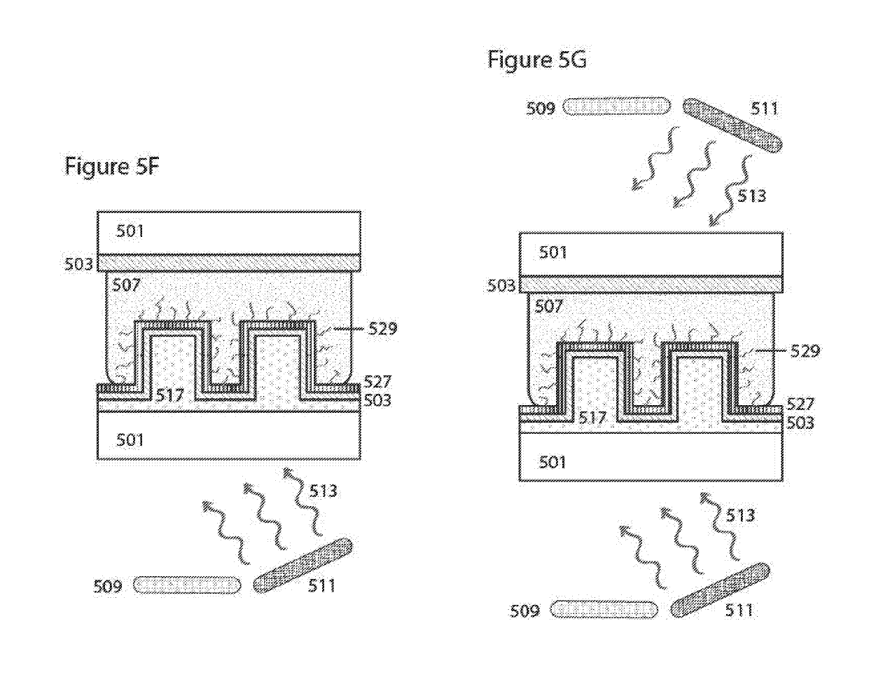

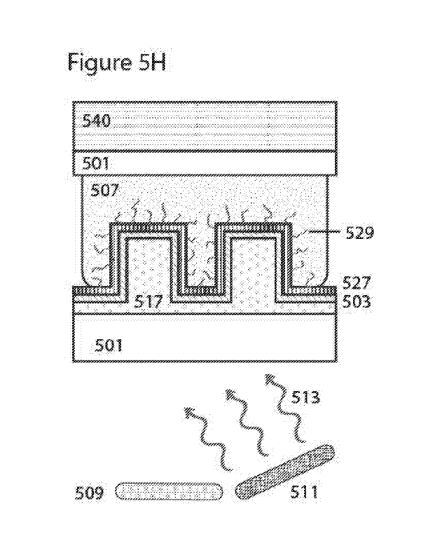

The chamber, well, and channel configurations discussed in the previous embodiments can readily be utilized for liquid-phase PCR, in which the amplification reaction occurs in the bulk of the solution. All of these configurations can also be utilized in solid-phase PCR, in which one or more set of primers are attached to a solid surface causing the amplification process to occur near or on the solid surface to which the primers are attached. The embodiment in FIG. 5A shows a cut-away illustration of an example reaction zone composed of a substrate 501 with 3D pattern layer 517 and heat generation layer 503 patterned and deposited on its surface. Linker layer 527 is composed of intermediate chemical molecules for immobilization of nucleic acid primers modified with matched linking chemical molecules for covalent binding to the linker layer. Linker layer 527 is shown to be modified on top of the heat generation layer 503 in the embodiments of FIG. 5. However, it is to be understood that linker layer 527 may be modified to any of the subsequent layers which can be deposited on the heat generation layer 503, as described in previous embodiments. For example, linker layer 527 may be deposited on top of the passivation layer which may be deposited on the heat generation layer. Linker layer 527 may be composed of one or more molecules, such as a functional silane, which may then be subsequently modified with one or more chemical linkers to impart certain functionality to the surface of the silane layer. For example, the linker layer can comprise a layer of APTES silane, with amine functionality, which can be used to bind primers modified with NHS-esters. Alternatively, the APTES layer may be reacted with succinic anhydride, generating a carboxyl functionality which may be used to bind amine-terminated primers. The choice of specific chemicals could depend on the application, among other considerations. Nucleic acid primers 529 are shown attached to linker layer 527. It is to be understood that the embodiment in FIG. 5A illustrates only one example configuration of a reaction zone in which solid-phase PCR could take place. The reaction zone is shown in FIG. 5A has a 3D pattern but a planar surface may also be used in lieu of a 3D pattern.

The attachment of primer to or near the solid surface of the heat generation layer or subsequently deposited layers may be accomplished by different methods. The embodiments in FIGS. 5B-E show cut-away illustrations describing a variety of methods to immobilize primers near the solid surface on the reaction zone. The embodiment in FIG. 5B shows a cut-away illustration of an example solid surface, wherein nucleic acid primers 529 are physically bound directly to the linker layer 527. The embodiment in FIG. 5C shows a cut-away illustration of an example solid surface, wherein nucleic acid primers 529 are chemically bound to the linker layer 527 via intermediate chemistry layer 531. To address possible steric hindrance issues, the nucleic acid primers may be attached a small distance away from the solid surface. This can be achieved by using a mesh of molecules like, for example, dextran or PEG, or with a physical layer such as beads. The embodiment in FIG. 5D shows a cut-away illustration of an example solid surface, wherein nucleic acid primers 529 are bound to the linker layer via a polymer brush 533, such as carboxymethyl dextran, which is covalently attached to the linker layer 527. The embodiment in FIG. 5E illustrates a method to immobilize nucleic acid primers close to the solid surface by using beads or particles 535 modified with linking molecule(s) 531. The beads/particles should be able to withstand temperatures encountered in a typical PCR reaction, up to approximately 95° C. Therefore, the beads may be plastic or polymer (for example, melamine), glass (for example, SiO2), metal or metal oxide (for example, magnetic beads). The beads 535 can be immobilized on the solid surface via covalent binding of linking molecules 531 on the beads to the linker layer 527 on the solid surface. The configurations illustrated in the embodiments in FIGS. 5D and 5E could alleviate possible steric hindrance issues while retaining close proximity of the primers to the heat generation layer, such that the temperature of the solution near the primers is identical to or very close to the temperature of the solid surface (for example, the heat generation layer). In a test cartridge having one or more reaction zones where PCR occurs, one or any combination of methods described in the embodiments in FIGS. 5B-E may be used to immobilize primers near the surface of the reaction zone(s). In each such reaction zone of the reaction region on the test cartridge, there would be many immobilized primers on one or more support structures. For example, a particular reaction zone may have a layer of multiple polymer brush molecules (shown as a single molecule in FIG. 5D for illustration) or a multitude of modified beads or particles (shown as a single bead in FIG. 5E for illustration).

The embodiments in FIGS. 5F-H show cut-away illustrations of three example configurations of a reaction zone for solid-phase PCR utilizing the channel/chamber format and leveraging a 3D pattern layer of enhanced performance and reaction speed. In the embodiments in FIGS. 5F, 5G, and 5H, the bottom substrate 501 is configured with a 3D pattern layer 517 with heat generation layer 503 deposited on its surface. Linker layer 527 is modified on heat generation layer 503 for immobilization of nucleic acid primers 529. The heat generation layer on the bottom substrate is heated by light output 513 from light source 511 and temperature is measured by thermal sensor 509. In the embodiment in FIG. 5G, another light source is used to heat the heat generation layer 503 on the top substrate such that both heat generation layers, on the top and bottom substrates, generate heat. In the embodiment in FIG. 5H, a supplementary heating device or element (for example, polyimide or silicone foil heater) is used to heat the top substrate 501, while the bottom substrate and the heat generation layer deposited on it are heated by light source 511. A thermal sensor (not shown) can be physically attached to the supplementary heating device or between the supplementary heating device and the top substrate, to monitor and regulate the temperature of the top substrate.

The three configurations illustrated in FIGS. 5F-H enable different methods to perform solid-phase PCR. In one method, using the configuration illustrated in the embodiment in FIG. 5F, the PCR mix solution 507 is heated to the different temperatures used in PCR by the heat generation layer 503 on the bottom substrate, which is exposed to light from the light source 511 to facilitate heating. As a result, only the temperature of the heat generation layer 503 on the bottom substrate is varied to reach the different temperatures used during PCR (denaturing, annealing and extension temperatures). One key advantage of using an optically excited heat generation layer for solid-phase PCR is that it is not necessary to ensure that the bulk of the PCR mix solution 507 reaches the desired temperatures in a PCR reaction because the amplification reaction occurs only near the solid surface (for example, the heat generation layer) during solid-phase PCR. Technically, for successful solid phase PCR reaction to occur, only the temperature of a thin layer of PCR mix solution near its surface needs to be changed or cycled. Therefore, the effective thermal mass which needs to be heated and cooled is only the heat generation layer and a thin layer of PCR mix solution near its surface. Using an optically excited heat generation layer makes this process rapid and efficient because it allows localized selective spot-heating of the surface of the reaction zone(s) without expending energy to unnecessarily heat large portions of the cartridge or substrate etc.

In another embodiments, the method is disclosed herein using the configuration illustrated in embodiment in FIG. 5G. The PCR mix solution 507 is heated by heat generation layers 503 on both the top and bottom substrates 501 by separate light sources. This configuration allows differential heating from the top and bottom for applications which might need the PCR mix solution to be uniformly heated during PCR. Both the top and bottom heat generation layers 503 can be heated to the same temperatures during PCR cycles, ensuring uniform heating of the PCR mix solution. Alternatively, the heat generation layer 503 on the top substrate 501 could be used to heat the PCR mix solution to a certain temperature while heat generation layer 503 on bottom substrate 501 could be used to flash-heat the bottom to a different temperature. For example, in a typical two-step PCR process, temperature is cycled between primer anneal and denaturing temperatures. Using the configuration shown in the embodiment in FIG. 5G, the heat generation layer 503 on top substrate 501 can be used to the maintain the temperature of the bulk PCR mix solution at the primer anneal temperature while the heat generation layer 503 on bottom substrate 501 can be used to rapidly flash-heat the surface of the heat generation layer and a thin layer of solution near its surface to the extension and/or denaturing temperatures. The temperature ramp rate can be well controlled by measuring the temperature of the heat generation layer on the bottom substrate with thermal sensor 509 for closed loop control of the light source. Because the temperature of the bulk PCR mix solution does not have to be cycled uniformly through all different temperatures, overall test time is reduced. Similar to the embodiment in FIG. 5G, the configuration shown in the embodiment in FIG. 5H achieves differential heating between the top and bottom substrates as well. However, in the configuration shown in the embodiment of FIG. 5H, the top substrate and the bulk PCR mix solution is heated with a supplementary heating device in contact with the top substrate. Temperature cycling only occurs in the thin region of solution near the surface of the heat generation layer 503 on bottom substrate 501 via flash-heating by light source 511.

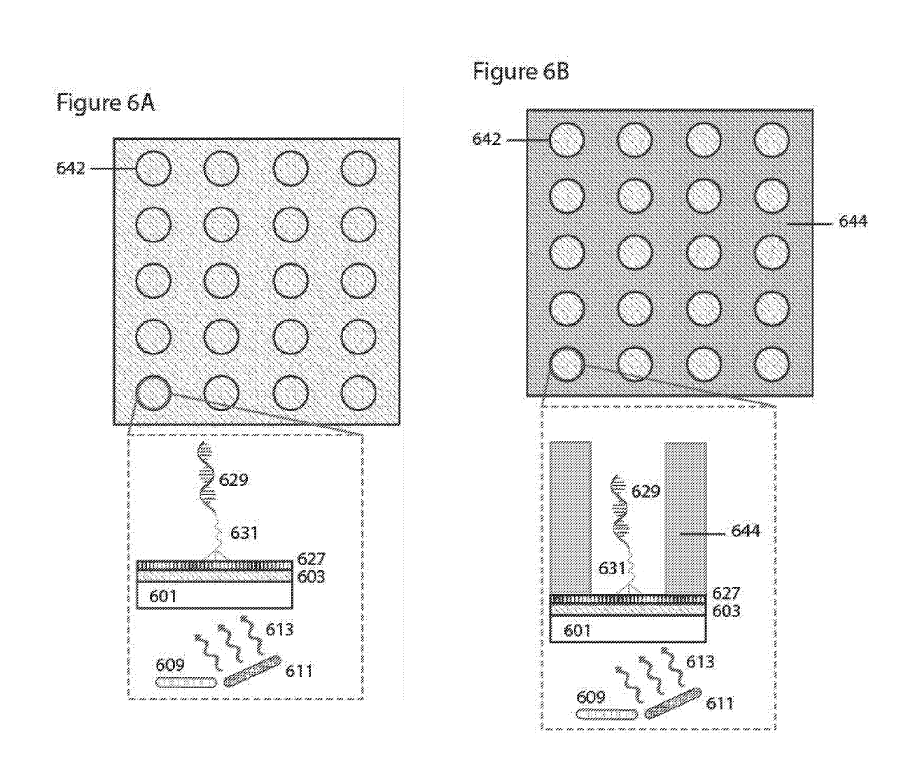

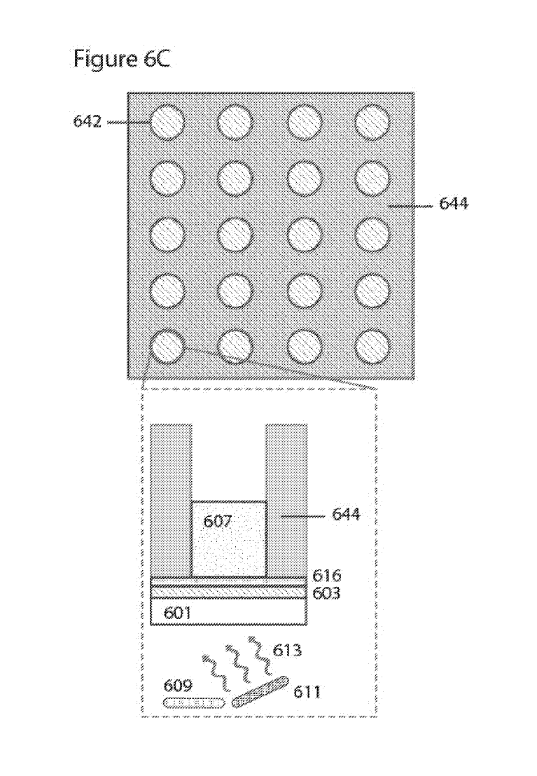

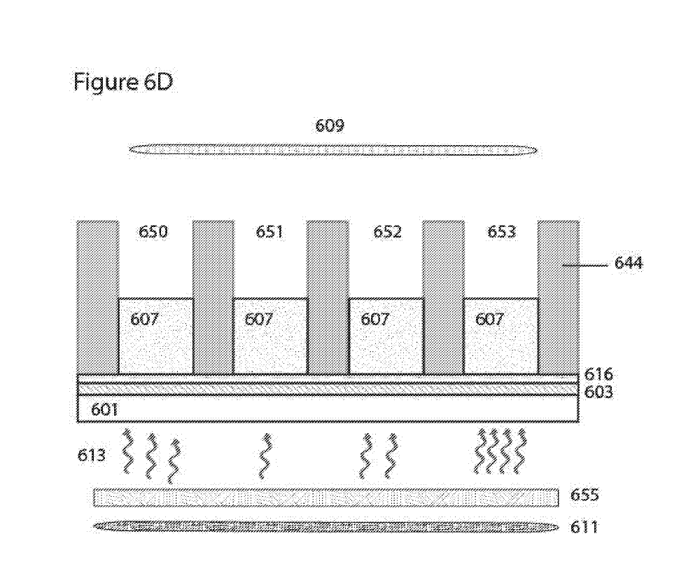

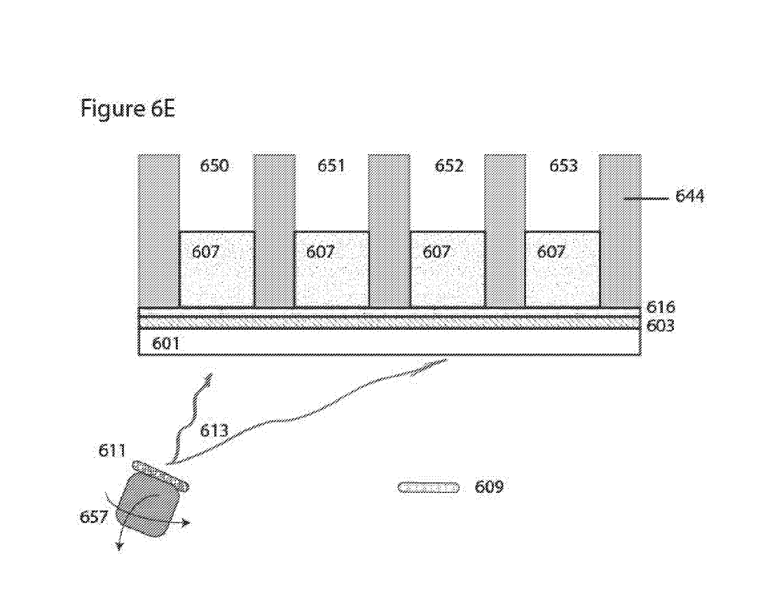

The embodiment in FIG. 6A illustrates an example of an array of reaction zones 642 in the reaction region of a test cartridge. The surface of each reaction zone can be modified with one or more sequences of nucleic acid primers 629. A different set of primers sequences can be modified in each reaction zone, enabling detection of a total of 20 target sequences from a sample. The size of the array of reaction zones 642 can be adapted to detect any number of sequences required in an application. For example, the array of reaction zones 642 in this embodiment contains 20 reaction zones and is capable of detecting 1 to 20 unique target sequences in the sample. The cut-away illustration in the inset of FIG. 6A shows an example of primer immobilization on the surface of one reaction zone for solid-phase PCR. In this inset, primer 629 is immobilized to the surface of the reaction zone and directly atop the heat generation layer 603 on substrate 601 via a combination of surface chemistry layers 627 and 631. The embodiment in FIG. 6B illustrates that the region in between individual reaction zones 644 may be raised above the surface of each reaction zone, such that the surface of the reaction zone forms the bottom of wells or channels into which PCR mix solution is injected and can be localized and contained. Wells or channels may be made by forming, patterning or placing a patterned layer of plastic, polymer, resist or oxide on top of the reaction zones. Wells can be formed by fabricating the patterned layer such that it is etched away on top of the circular reaction zones, leaving only the surface of the reaction zones exposed. Alternatively, channels may be formed by etching away rectangular strips exposing rows or columns of reaction zones and the intermediate space between the sequential reaction zones in each row or column. Another way to make such wells or channels on top of the array of reaction zones is to adhere or bond a pre-cut plastic insert on top of the array of reaction zones. The cut-away illustration in the inset of FIG. 6B shows an example of primers immobilized on the surface of one reaction zone at the bottom of such wells or channels for solid-phase PCR. In this inset, primer 629 is immobilized to the surface of the reaction zone, directly atop the heat generation layer 603 on substrate 601 and in the well or channel formed by pattern layer 644 via a combination of surface chemistry layers 627 and 631. This configuration of an array of individually separated reaction zones, as illustrated in the embodiments in FIG. 6, may also be utilized for liquid-phase PCR. The embodiment in FIG. 6C illustrates an array of reaction zones 642 having an optically excited heat generation layer configured for liquid-phase PCR. The individual reaction zones in the array of reaction zones 642 are isolated by 3D pattern layer 644 configured as an array of well structures in this example embodiment. The inset of FIG. 6C shows a cross sectional illustration of an example reaction zone for liquid-phase PCR. Heat generation layer 603 on substrate 601, with an optional passivation layer 616 on the heat generation layer 603. Passivation layer 616 may be oxide, plastic, silicone or polymer. Alternatively, if using a dye or pigment as heat generation layer, it is possible to integrate the heat generation layer into the substrate by pre-infusing the substrate with absorptive dye or pigment or by depositing the dye or pigment on either side of the substrate 601 or by adhering/bonding another transparent substrate with the pigment/dye onto substrate 601. The 3D pattern layer 644, a well structure in this embodiment, holds PCR mix solution isolated in the well and above the heat generation layer, constituting a reaction zone in which liquid-phase PCR can be performed. The temperature of the heat generation layer 603 and/or PCR mix solution 607 may be monitored by a non-contact infrared or contact thermal sensor 609. As in previous embodiments, light output 613 from light source 611 excites heat generation layer 603, which transfers thermal energy into solution.

The temperature of the surface of each reaction zone in the array of reaction zones 642 can be controlled simultaneously by ensuring that light source 611 exposes all of the reaction zones in the array with light output 613 having a known and/or uniform intensity distribution. Alternatively, a liquid crystal optical component, such as a chip-on-glass liquid crystal device, placed between the light source and the substrate may aid in discretely controlling intensity of light output under each reaction zone in the array. The light output passing through each liquid crystal pixel can be controlled by controlling the frequency at which the liquid crystal is switched on and off, allowing for a different light intensity to reach the bottom of each reaction zone in the array. The can also be achieved using a DLP mirror chip. The embodiment in FIG. 6D illustrates a cross sectional view of four reaction zones 650-653 from a single row of an array of reaction zones. Light output energy 613 to each reaction zone is individually controlled by a chip-on-glass LCD optic 655 placed beneath substrate 601 such that groups of pixels in the LCD chip are activated separately to control the intensity profile of the light reaching the heat generation layer 603 under each reaction zone in the array. In this manner the LCD component can be used to both provide different intensity of light to each reaction zone in the array while using a single light source. This method can also allow for correction for non-uniformity in the output of the light source (for example, correcting for Gaussian light intensity profile from a LED source) to provide light of uniform intensity to each reaction zone in the array. A non-contact thermal sensor 609 is used to monitor the temperature of each reaction zone from the top, as it is heated. Alternatively, a targeted energy source with a narrow coherent beam such as a laser diode can be scanned over the array of reaction zones, stopping the beam under each reaction zone for an amount of time needed for the heat generation layer to reach the desired temperature. The embodiment in FIG. 6E illustrates a cross sectional view of four reaction zones 650-653 from a single row of an array of reaction zones. Light output beam 613 is scanned over each reaction zone in the array such that the beam exposes each reaction zone for a length of time needed to reach the desired temperature, as measured by non-contact thermal sensor 609. A steering device 657 is used to mechanically or electronically steer light beam 613, output from light source 611, which is attached to the steering device 657. The steering device 657 may be an electro-optical beam steering module or a physical galvanometer device. Either of the methods described in the embodiments in FIGS. 6D and 6E can be used to control the temperature of the surface of each reaction zone (and the temperature of the solution in each reaction zone) individually, thereby allowing customized and separate PCR reactions to occur in each reaction zone of the array.

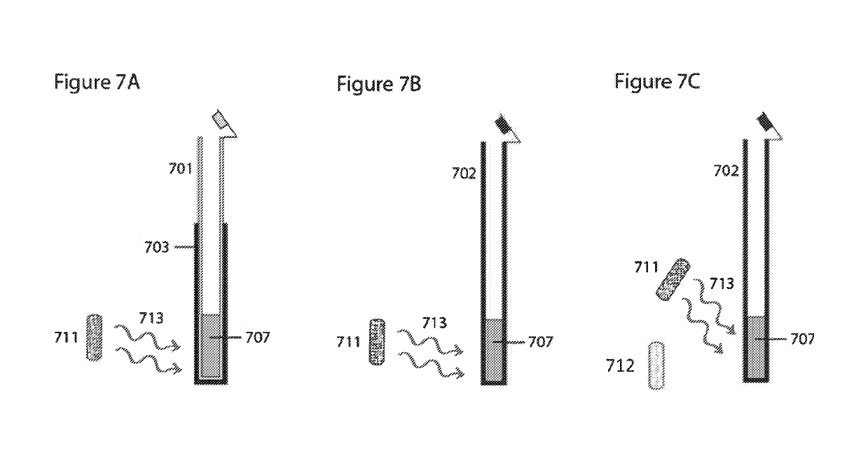

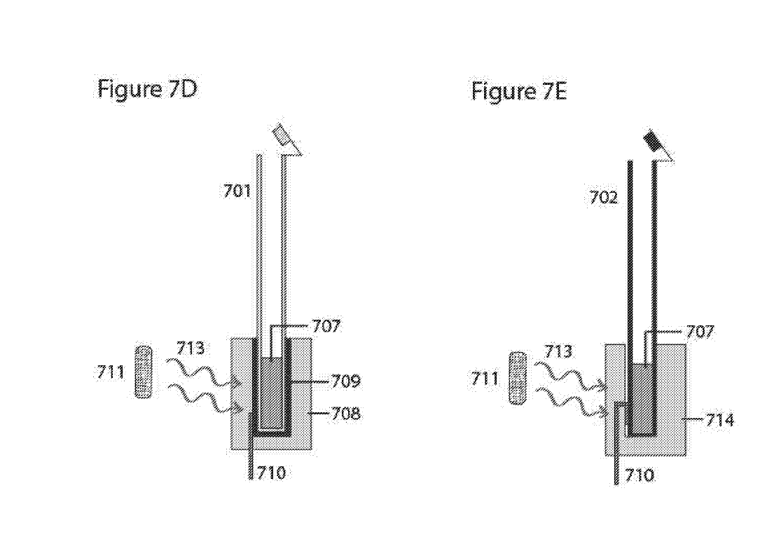

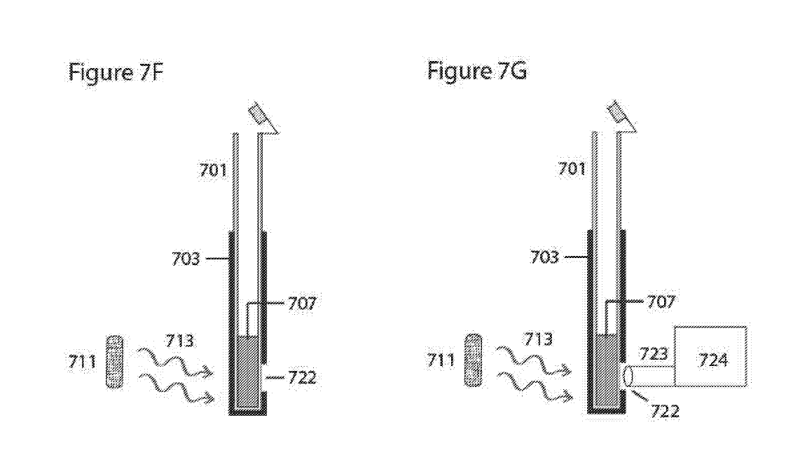

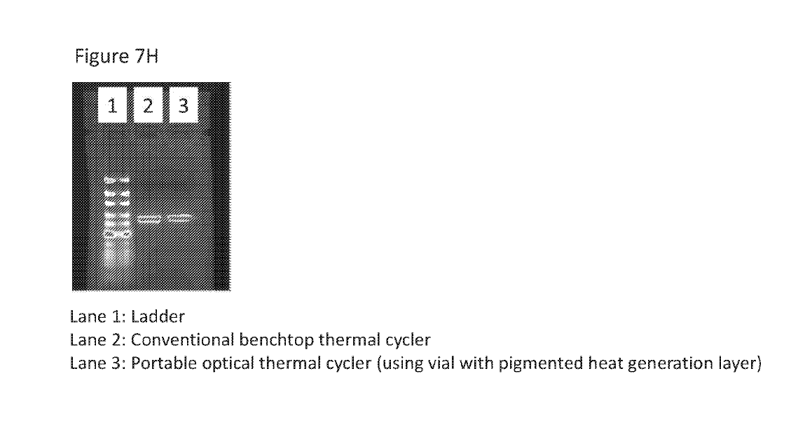

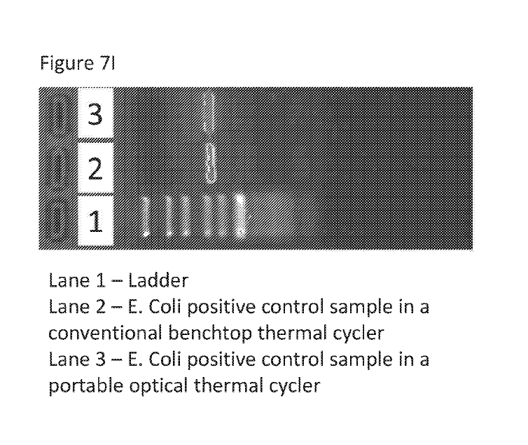

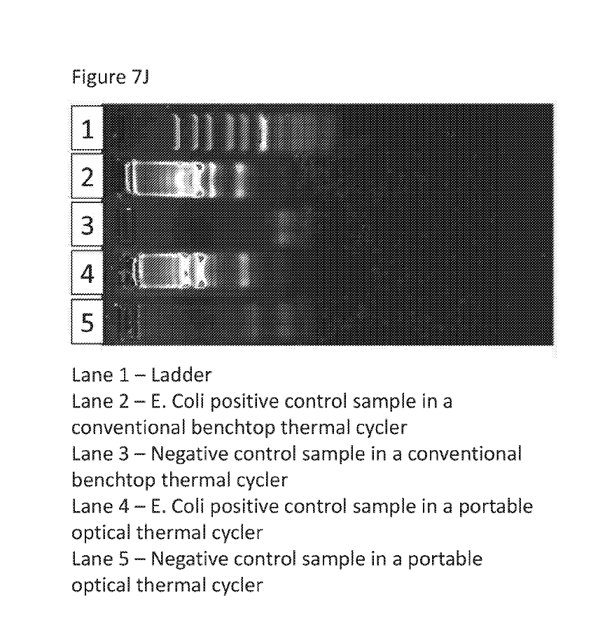

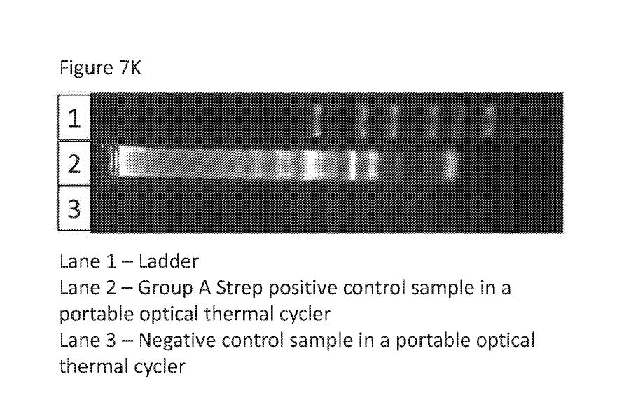

Although localized spot-heating by optical energy may be applied to PCR application using a cartridge format, a plastic or glass vial may also be used as a container for PCR mix solution and the PCR reaction. In conventional laboratory thermal cyclers, liquid phase PCR reactions are typically performed in plastic vials heated uniformly by large heat blocks. These thermal cyclers are bulky and demand significant power requirements. A plastic vial can be adapted to make use of a heat generation layer that generates heat upon optical excitation, allowing heating of the vial by a compact low-power light source in lieu of a large heat block. The embodiments in FIG. 7 show several configurations of a vial adapted to allow optical heating of the PCR mix solution in the vial. In order to heat solution inside a vial, the outer surfaces of the vial can be coated with a heat generation layer, similar to the surface of the reaction zone(s) in the reaction region of a test cartridge, as described in previous embodiments of this disclosure. The heat generation layer can comprise any of the aforementioned materials discussed in this disclosure (for example, plasmonic metal, semiconductor, optically absorptive polymer, or pigments and/or dyes). For example, a thin layer of germanium may be deposited or coated on at least a portion of the outer surfaces of the vial. Polyimide film, such as Kapton® tape, may be adhered to at least a portion of outer surfaces of the vial. In a simpler method, a layer of pigment or dye can be directly coated on at least a portion of the outer surfaces of the vial. If a portion of the vial is coated, the portion onto which optical energy is incident by the light source is preferably coated. The vial is to be adequately thin, having a large surface area-to-volume ratio. The inner volume of the vial should be configured to allow a continuous or uniform thin layer of liquid to be contained within the inner sidewalls of the vial such that incremental liquid volume is accommodated by increasing vial length, while maintaining the same small cross-sectional area. The vial should preferably have a thin diameter or cross-sectional area to ensure heat is transferred uniformly and rapidly from the portion of the outer surface of the vial heated by optical energy to the bulk of the solution inside the vial. Therefore, a thin and long vial, with a uniform and small rectangular or circular cross-section area is preferred. The total length of the vial can be adjusted based on the volume of PCR mix solution used for an application. The vial is enclosed on the bottom and has a removable cap on the top, which can be opened to inject liquid and closed to seal the vial during reaction. The embodiment in FIG. 7A illustrates one possible configuration of a vial designed for PCR reaction with an optically excited heat generation layer. The outer surfaces of vial 701 are coated with a heat generation layer 703, comprised of black pigment in this embodiment; the black pigment covers at least the area of the vial to be exposed to optical energy for heating or may cover the entire outer surface of the vial. At least a portion of the outer surface of the vial coated with heat generation layer 703 is excited with light output 713 from energy source 711 to produce the thermal energy needed for PCR reaction to occur in PCR mix solution 707 contained within the vial. Energy source 711 can be a light source such as a laser diode or LED, similar to previous embodiments. The embodiment in FIG. 7B shows another configuration in which the vial 702 is molded or constructed from plastic stock or pellets which are pre-infused or dyed with black pigment, which serves as the heat generation layer, thereby not requiring a secondary coat of pigment on the outer surfaces of the vial. As in the embodiment in FIG. 7A, the vial contains PCR solution mix 707 and is heated by light output 713 from energy source 711. Localized optical heating of the PCR mix solution in the vials, via the heat generation layer, enables novel and unconventional form-factors for portable DNA diagnostics. Non-contact optical heating of PCR mix solution inside such vials can enable compact and low-power portable equipment for DNA diagnostics because a typical LED or laser diode which can be used to excite the heat generation layer of the vial is significantly smaller and more power-efficient than conventional heat blocks or heating elements.

To monitor and track temperature of the vial as it is heated a contact or non-contact thermal sensor may be used. The embodiment in FIG. 7C illustrates one configuration in which a non-contact thermal sensor is used to monitor temperature of the vial 702. The PCR mix solution 707 in vial 702, constructed from pre-dyed or pre-pigmented plastic, is heated by light output 713 from energy source 711. Thermal sensor 709 monitors the temperature of the surface of the heated portion of the vial by measuring the infrared radiation output by the heated surface. Thermal sensor 709 may be a thermopile, bolometer sensor chip or focal plane array, or a pyrometer. Thermal sensor 709 monitors the temperature of the heated surface continuously or within short intervals to provide feedback enabling real-time closed-loop control of the intensity of light output 713 from energy source 711 for temperature cycling during the PCR reaction. The embodiments in FIGS. 7D and 7E illustrate methods to use a contact temperature sensor to monitor the temperature during an optically-enabled PCR reaction in a vial. The embodiment in FIG. 7D illustrates a configuration for localized optical heating of an unmodified vial containing PCR mix solution 707 via an optically excitable plastic heating block into which an unmodified vial can be inserted. An unmodified vial 701, containing PCR mix solution 707, is inserted into the plastic heating block 708 for PCR reaction. The inner surface of the plastic heating block 708, which contacts the outer surface of vial 702 directly, is coated with heat generation layer 709, for example, black pigment in this embodiment. The vial may also be modified with the same or another heat generation layer, but is unmodified in this embodiment. A thin-gauged micro-thermocouple sensor 710 is connected to the heat generation layer 709, exposed to light output 713 from energy source 711, to monitor the temperature of the heat generation layer 709. The temperature of the surface of the vial would closely match that of the heat generation layer 709 on the plastic heating block 708. The thermocouple sensor may be inserted and affixed into a hole in the plastic heating block such that it lies directly on the heat generation layer 709 or in close proximity to it for accurate temperature measurement. The embodiment in FIG. 7E illustrates a configuration in which a plastic block without heat generation layer is used in conjunction with a vial having a heat generation layer. Vial 702 containing PCR solution mix 707 is inserted into a plastic block 713. Vial 702 is made with plastic containing heat generation material, such as for example, black pigment, as in some of the previous embodiments. The heat generation material in vial 702 is optically excited by light output 713 from energy source 711. Plastic block 714 is configured to have a hole or slot cut into it through which a thin-gauged micro-thermocouple 710 is inserted. Thermocouple 710 is inserted into plastic block 714 and affixed in place such that the hot junction of the thermocouple touches the heated surface of vial 702 in order to monitor the surface temperature of the heated vial. In this configuration the plastic block is only used to hold the thermocouple while heating is facilitated by the heat generation layer on the vial. In any configuration of the system in which a contact-based thermal sensor is used, the thermal sensor can be a thermocouple, thermistor, RTD, or semiconductor temperature sensor configured such that it contacts or is in close proximity to the heated surface of a plastic heating block or the heated surface of the vial.

In certain protocols requiring real-time or end-point fluorescence detection after PCR, it may be preferential to use a vial which has been coated with heat generation layer or material. The embodiment in FIG. 7F shows a configuration in which fluorescence output of the PCR mix solution is monitored during or after the PCR process. Vial 720 is coated with heat generation layer 703, for example, black pigment, such that a portion 722 of the vial is left uncoated or has the coating of heat generation material removed such that PCR mix solution 707 is visible through opening 722. This exposed region 722 can be used to monitor the fluorescence output of the PCR mix solution during or after the PCR process. An example configuration for detection fluorescence output using the vial shown in the embodiment in FIG. 7F is illustrated in the embodiment in FIG. 7G. Vial 701 is configured with a heat generation layer 703 having an opening 722 in the heat generation layer exposing PCR mix solution 707. A fluorescence sensor unit 724 can be interfaced to the opening 722 by a fiber optic cable or fiber optic cable bundle 723 to optically excite PCR mix solution 707 and measure its fluorescence output. The fluorescence sensor unit 724 may contain miniature optics and sensors for fluorescence detection such as a LED light source, optical components and a photo-detector or miniature spectrometer chip etc. This type of configuration would enable rapid real-time PCR or quantitative PCR in a low-power ultra-compact portable format using a vial for PCR reaction.

FIG. 7H shows gel electrophoresis results of an experiment in which a 90 base pair section of lambda DNA is amplified in a conventional bench-top thermal cycler vs. a prototype portable optical thermal cycler using a vial having an optically excited heat generation layer. A polypropylene PCR vial coated with black pigment, similar to the vial shown in the embodiment in FIG. 7A, is used in the portable optical thermal cycler. For use in the portable optical thermal cycler, a PCR vial identical to the vial used in the bench-top cycler is painted with black Sharpie® pigment on the outer surfaces of the vial to create an optically excitable heat generation layer on the surface of the vial. The prototype portable optical thermal cycler contains an LED for heating while the temperature of the surface of the vial is measured with an ultra-thin gauge thermocouple adhered to the heated surface of the vial. As seen in FIG. 7H, amplification of target DNA in the portable optical thermal cycler, performed using a vial with a pigmented heat generation layer, is very similar to that of the conventional bench-top thermal cycler. This clearly demonstrates the viability of using a vial with pigmented heat generation layer with localized spot-heating by optical energy for thermal cycling during PCR.

While FIG. 7H demonstrates the feasibility of optical heating for thermal cycling in PCR, contact-based temperature measurement using a thermocouple may present a challenge in product integration and efficacy. Temperature measurement via thermocouple requires integration of a thermocouple onto the surface of the vial, inside the vial such that the thermocouple touches the PCR mix solution, or into the fixture where the vial is inserted—all these methods are cumbersome, costly and require perfect contact between the thermocouple and the vial while only providing temperature data from a small finite point. A contactless temperature sensor, such as an infrared (IR) sensor 712 (as shown in the embodiment of FIG. 7C), provides simultaneous area-based averaged temperature data from the heat generation layer that is heated by the light source, thereby improving accuracy of the closed-loop feedback system which controls the intensity of the light source. Furthermore, the IR sensor 712 also facilitates easier integration into an instrument while making the instrument easier to use for the end-user by relaxing constraints on insertion force and configuration. FIG. 7I shows gel electrophoresis results of an experiment which compares DNA amplification in a conventional heat-block based benchtop thermal cycler and a prototype portable optical thermal cycler with a configuration similar to the embodiment of FIG. 7C. A 96 base-pair long E. coli target sequence DNA is amplified in a black pigment coated polypropylene PCR vial as shown in the embodiment in FIG. 7C. The prototype portable optical thermal cycler used for this experiment employs an LED source 711 for heating the black pigmented heat generation layer on the surface of the vial. Surface temperature of the area of the vial heated by the LED source is measured by a thermopile IR sensor 712, as in the configuration shown in the embodiment of FIG. 7C. Similar to the thermocouple sensor 710, the IR sensor 712 provides closed-loop feedback that enables real-time control of the intensity of light output 713 from LED source 711; however, the IR sensor 712 provides more accurate temperature measurement of the full area excited by the LED light source, resulting in improved accuracy with less complexity. As seen in the FIG. 7I, the target DNA amplification in the portable optical thermal cycler using IR sensor 712 is comparable to both portable optical thermal cycler with thermocouple sensor 710 as well the conventional benchtop thermal cycler, which employs a heat-block for heating. This clearly elicits the advantage, efficiency and efficacy of localized spot-heating by an LED source regulated by closed-loop feedback from a contactless temperature sensor for DNA amplification in an optical thermal cycler.

Loop mediated isothermal amplification (LAMP) is an effective isothermal alternative to traditional PCR for DNA amplification. Driven by free energy and relative stability, LAMP reactions can be carried out at a constant temperature and result in concatenated DNA amplicons that are characterized by a ladder-like structure on the gel. In comparison to PCR, sample preparation for LAMP is quicker and less complicated because LAMP reactions are significantly less sensitive to impurities and other components in the reaction mix. Moreover, as LAMP reactions are performed at constant temperature, overall assay time is also improved as compared to traditional PCR because of the elimination of thermal cycling. The optical heating method is particularly well suited to LAMP reactions because of the inherent low-power capability, portability and ease-of-use offered by non-contact closed-loop heating in the small portable form-factor of the optical thermal cycler. FIG. 7J shows gel electrophoresis results of an experiment that compares DNA amplification in a conventional heat-block based benchtop thermal cycler and a prototype portable optical thermal cycler for E. coli target DNA amplification using LAMP. It is clear from FIG. 7J that a LAMP reaction performed in the prototype portable optical thermal cycler with contact-less IR sensor 712, similar to the configuration shown in the embodiment in FIG. 7C, produces results analogous to the benchtop thermal cycler. Isothermal DNA amplification using LAMP employs a simpler sample preparation and thermal protocol, making LAMP a favorable technique in portable systems. Non-contact closed-loop optical heating for thermal control synergizes particularly well with LAMP for implementation of simpler and/or portable DNA amplification and detection systems.

FIG. 7K shows gel electrophoresis results in which a portable optical thermal cycler is used to amplify a target gene of Group A Streptococcus (GAS) using LAMP. The results demonstrate that the portable optical thermal cycler is a versatile tool capable of DNA amplification of any target using a variety of thermal protocols. The prototype portable optical thermal cycler performs DNA amplification as well as a conventional heat-block based benchtop thermal cycler, but with significant advantages. The power-consumption, size, and cost of the prototype portable optical thermal cycler are significantly lower than a conventional benchtop thermal cycler. Non-contact optical heating and temperature monitoring in the portable optical thermal cycler reduces complexity, making it more robust and easier to use. Furthermore, the prototype portable optical thermal cycler further simplifies the implementation of LAMP by reducing complexity of instrumentation compared to typical heat-block enabled thermal cyclers. Therefore, pairing optical thermal control with LAMP leverages the favorable characteristics of the portable optical thermal cycler, making it possible to build small and inexpensive, yet effective DNA diagnostic consumer products.

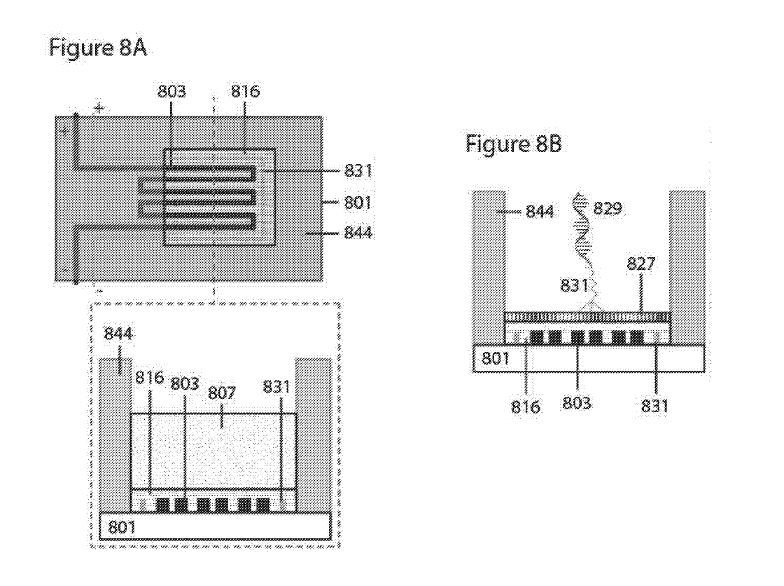

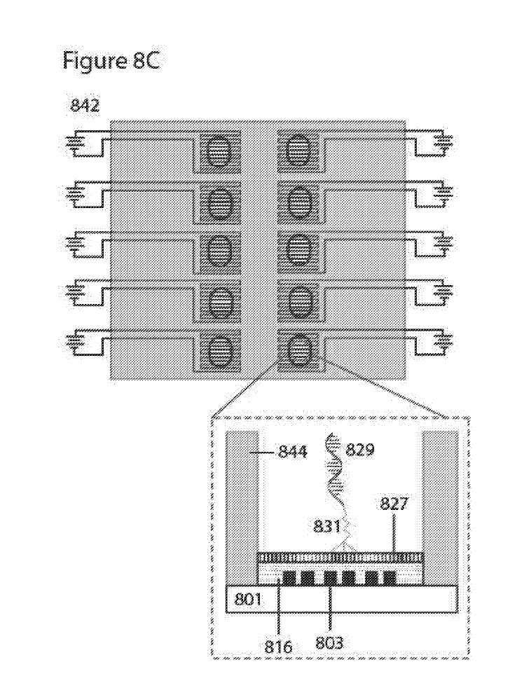

Referring again to the cartridge configuration, another method of targeted localized heating of PCR mix solution in the reaction zone(s) of a test cartridge is spot-heating using resistive heater circuits fabricated directly on the reaction zone(s) of the test chip or cartridge. The embodiment in FIG. 8A shows one configuration of a reaction zone with a resistive heater circuit fabricated underneath the reaction zone. Resistive heater circuit 803 is comprised of a spiral track made of metal, semiconductor or alloy, with either terminal of the track interfaced with power driving circuitry which flows electrical current through the track; the power driving circuitry can be made/placed on or off the same chip/cartridge. The flow of electrical current heats up the material which comprises the track, thereby transferring thermal energy into PCR mix solution 807. The resistive heater circuit 803 is fabricated directly onto the underlying substrate 801, which can be plastic, glass, or semiconductor. A thermistor circuit 831 is fabricated in the reaction zone or near the resistive heater circuit 803 to monitor the temperature of the solution near the resistive heater circuit. The thermistor circuit can be configured to interface with processing circuitry to apply current to the thermistor track and analyze the return current/voltage; the processing circuitry can be made/placed on or off the same chip/cartridge. Thermistor circuit 831 can be made from metal, semiconductor, allow or oxide. Passivation layer 816 is deposited onto the exposed substrate 801, resistive heater circuit 803, and thermistor circuit 831 to protect the surface and prevent interference with biological components in the solution in the reaction zone(s). Passivation layer 816 can be made from insulating oxide, plastic or silicone. Finally, 3D pattern layer 844 is fabricated or placed on around the reaction zone(s) so as to isolate individual reaction zone(s); 3D pattern layer 844 can be configured in a variety of structures, including wells, chambers, channels etc. 3D pattern layer 844 can be made from silicone, polymer, plastic or oxide. The inset of FIG. 8A shows a cross sectional view of the reaction zone in main embodiment of FIG. 8A, demonstrating the configuration of the individual layers and showing PCR mix solution 807 inside the 3D pattern layer 844, having a well structure in this example embodiment. Liquid phase PCR can be carried out in the PCR mix solution 807 in reaction zone(s), having resistive heater and optional thermistor circuits fabricated in each reaction zone. The temperature of the resistive heater circuit 803 and PCR mix solution 807 is monitored by the thermistor circuit 831 fabricated near the resistive heating circuit. Alternatively, the temperature can be monitored with a non-contact infrared thermal sensor placed under or above the cartridge such that it can measure the infrared emitted by the substrate or solution as it is heated by the resistive heater circuits. Monitoring temperature with either a non-contact infrared thermal sensor or built-in thermistor circuits allows closed-loop control of the current applied to each resistive circuit heater for control of temperature of each resistive heater circuit as well as the solution in each reaction zone. In a test cartridge having an array of reaction zone(s), each configured for detection of a different nucleic acid target, it may be advantageous to control the temperature of the solution in each reaction zone individually such that it is possible to carry out different PCR protocols (with varying temperature requirements) in each reaction zone simultaneously. Therefore, each resistive heater circuit under each reaction zone in the array of reaction zones can be driven with a different current to control the temperature of the solution in each reaction zone individually, as read by thermistor circuit 831 and/or a non-contact thermal sensor outside of the cartridge.

The configuration of the reaction zone discussed in the embodiment in FIG. 8A can be used for solid-phase PCR in each reaction zone. The embodiment in FIG. 8B illustrates a cross section of one reaction zone in an array of reaction zones configured for solid-phase PCR. Resistive heater and thermistor circuits, 803 and 831 respectively, are fabricated on top of substrate 801 and covered by passivation layer 816. The boundary of the reaction zone is defined by 3D pattern layer 844, configured in a well structure in this example embodiment. Linker layer 827, which may be a layer of chemical molecules, is deposited onto passivation layer 816. At least one set of nucleic acid primers 829 are covalently immobilized on the surface of the reaction zone's linker or passivation layer via interfacing chemical molecules 831. Rapid solid-phase PCR may be carried out in this configuration by maintaining solution temperature at primer annealing temperature using the resistive heater circuit or a supplementary heating device while rapidly flash heating the surface of the reaction zone and the thin layer of water above the surface, containing the primer strand, using the resistive heater circuit during the extension and denaturing steps in PCR. Because the nucleic acid primer strand is immobilized near the surface, only rapid flash heating of the thin layer of solution near the surface of the reaction zone is necessary to accomplish PCR using the primer immobilized on the surface of the reaction zone. As the bulk of the solution does not need to be heated uniformly during the extension and denaturing steps, the thermal mass is reduced, thereby reducing temperature cycling time. FIG. 8C illustrates an array of reaction zones 842 having individual heater circuits fabricated on each reaction zone of the array; the optional thermistor circuits are not shown, but can be added to each reaction zone in the array. The terminals of each resistive heater circuit are connected to driving circuitry which controls current flow through each resistive heater circuit (not shown). The inset of FIG. 8C shows a cross sectional view of a select reaction zone, in the array of reaction zones 842, configured for solid-phase PCR.

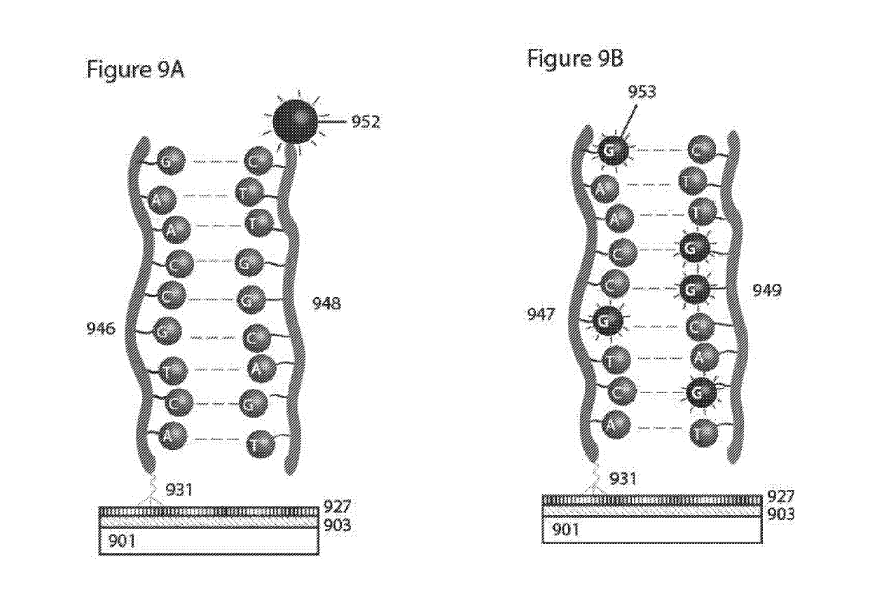

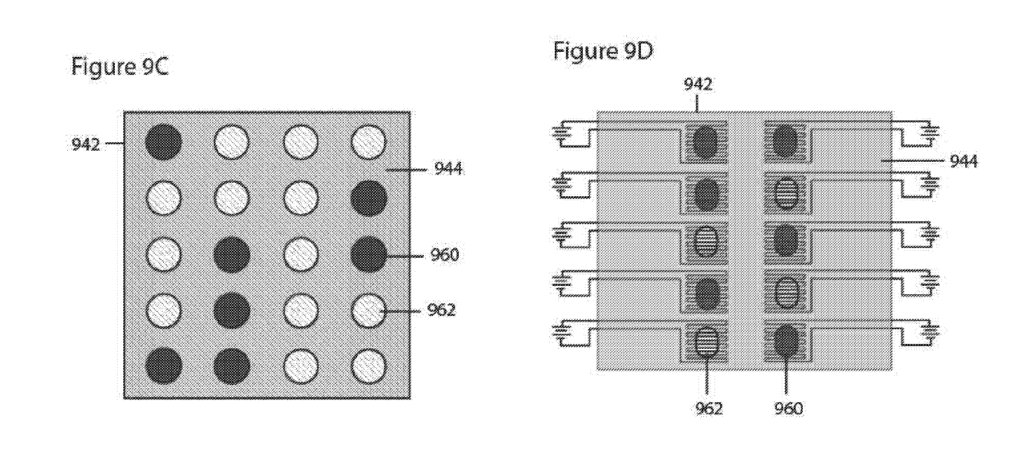

Numerous detection methods can be adapted for use in detecting DNA amplified in the reaction zone(s) of the cartridge(s) of the aforementioned embodiments. Fluorescence detection using probe primers having a fluorophore and quencher on opposite ends or intercalating dyes are commonly used and can be used in liquid-phase PCR performed with the cartridge(s) in the embodiments of this disclosure. However, multiplexing of more than a two to three targets is complex and costly in liquid phase PCR. Therefore, solid-phase PCR performed with the techniques discussed in previous embodiments, such as an array of reaction zones configured for solid-phase PCR, may be utilized as an alternative for highly multiplexed detection applications, such as disease panels where a single test should detect multiple diseases and/or pathogens or numerous serotypes and/or strains of a disease and/or pathogen. Solid-phase PCR with arrayed reaction zones, which are either individually or batch heated, is particularly suited to multiplexing as the different nucleic acid targets are physically separated in the array (each reaction zone in the array is modified with a different set of primers for detecting a different targets). Aforementioned fluorescence based methods which are used in liquid-phase PCR can also be used to detect the amplified strands extended on different surfaces (surface, polymer brush, beads/nanoparticles) during solid-phase PCR. Additionally utilizing fluorescently tagged primers which are then incorporated into the extended strands, in presence of target DNA template, can also be used. The embodiment in FIG. 9A shows the surface of an example reaction zone to illustrate a method for detection of amplified solid-phase PCR products. Solid-phase PCR is performed by heat cycling the thin region of liquid above the heat generation layer 903 on substrate 901 using optical energy to heat the heat generation layer 903. Before amplification, the surface contains at least a single set of primers, for example, forward primers, immobilized on the surface of the linker layer 927 via surface chemistry layer 931. The PCR mix solution above the surface of the reaction zone contains one or more sets of primers, with at least one type of fluorescently tagged primer, for example, reverse primers. Both forward and reverse strand extension take place on the surface during solid-phase PCR and result in amplicon strand 946 being extended from the immobilized set of primers and amplicon strand 948 being extended from the reverse primer in PCR mix solution, where the reverse primers have been covalently bound to fluorescent molecules 952. As a result of successful solid-phase PCR, the surface of the reaction zone will have many extended amplicons and will therefore be fluorescent. The embodiment in FIG. 9B shows the surface of an example reaction zone to illustrate another method for detection of amplified solid-phase PCR products. Solid-phase PCR is performed by heat cycling the thin region of liquid above the heat generation layer 903 on substrate 901 using optical energy to heat the heat generation layer 903. Before amplification, the surface contains at least a single set of primers, for example, forward primers, immobilized on the surface of the linker layer 927 via surface chemistry layer 931. The PCR mix solution above the surface of the reaction zone contains one or more sets of primers as well as at least one set of fluorescently labeled nucleotides 953, “G” or guanine in this embodiment. During forward and reverse extension of both sense and anti-sense amplicons, 947 and 949, during PCR, a certain number of the fluorescently labeled nucleotides 953 are incorporated into the extended DNA strands, thereby causing the surface of the reaction to be fluorescent upon successful PCR. The embodiment in FIG. 9C shows an array of 20 reaction zones 942 configured for solid-phase PCR detection of 20 possible target nucleic acid sequences in a sample, using an optically excited heat generation layer. The surface of each reaction zone in the array is modified with a different set of primers in order to detect a different target. Each reaction zone in the array is isolated by 3D pattern layer 944, configured as a well structure in this embodiment. In this example, seven out of 20 target nucleic acid sequences are present in the sample, resulting in seven fluorescent spots like reaction zone 960, within the array of reaction zones 942 after the PCR process. The other 13 targets are not present in the sample, resulting in 13 dull spots like reaction zone 962, which do not exhibit any fluorescence after the PCR process. The embodiment in FIG. 9D shows an array of 10 reaction zones 942 configured for solid-phase PCR detection of 10 possible target nucleic acid sequences in a sample, using electrically heated reaction zones. The surface of each reaction zone in the array is modified with a different set of primers in order to detect a different target. Each reaction zone in the array is isolated by 3D pattern layer 944, configured as a well structure in this embodiment. In this example, six out of 10 target nucleic acid sequences are present in the sample, resulting in six fluorescent spots like reaction zone 960, within the array of reaction zones 942 after the PCR process. The other four targets are not present in the sample, resulting in four dull spots like reaction zone 962, which do not exhibit any fluorescence after the PCR process. In this manner, multiplexed detection of multiple targets can be achieved rapidly after a quick PCR process using a compact and low-power reader enabled by the optically excited and/or electrically activated heat generation layers.

Both PCR and detection can be facilitated without any optical components in the case of solid-phase PCR using reaction zones on a cartridge with electrically activated heat generation layers. This is achieved by opting for electrochemical detection of DNA rather than fluorescence-based readout. In order to facilitate electrochemical detection of amplified DNA targets on the surface of a reaction zone, a simple electrochemical circuit resembling an electrochemical cell can be fabricated on top of the reaction zone in conjunction with the resistive heater and/or thermistor circuits.

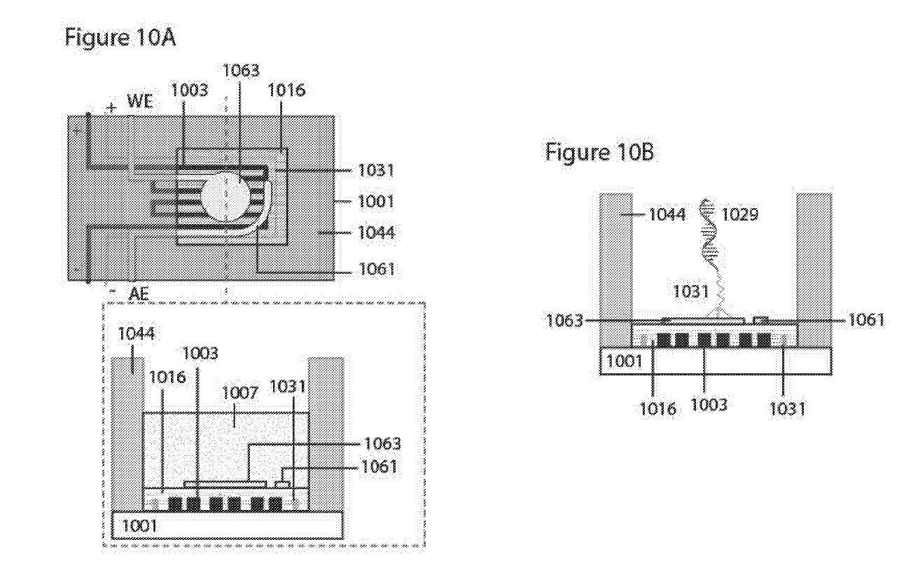

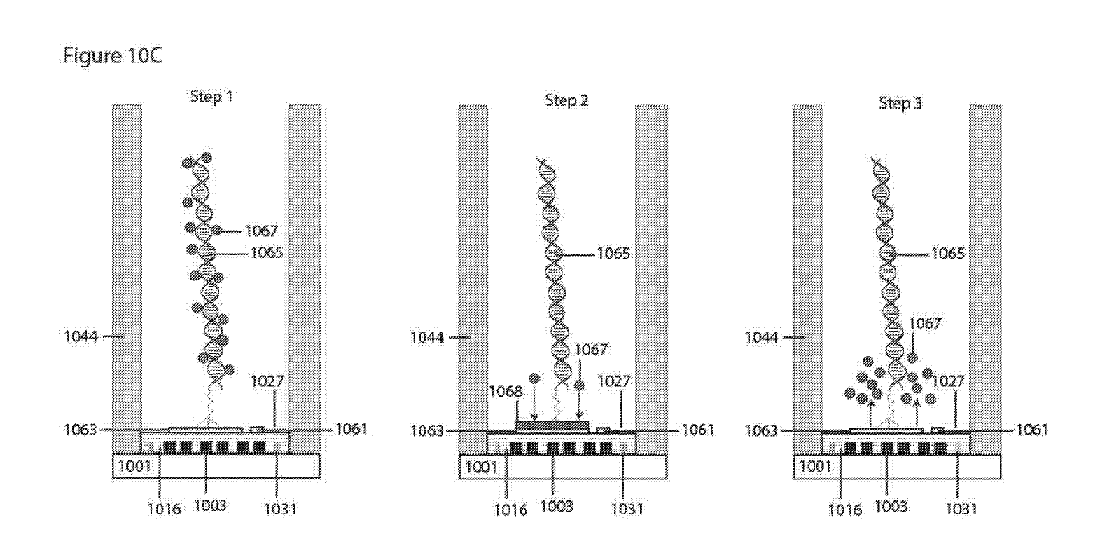

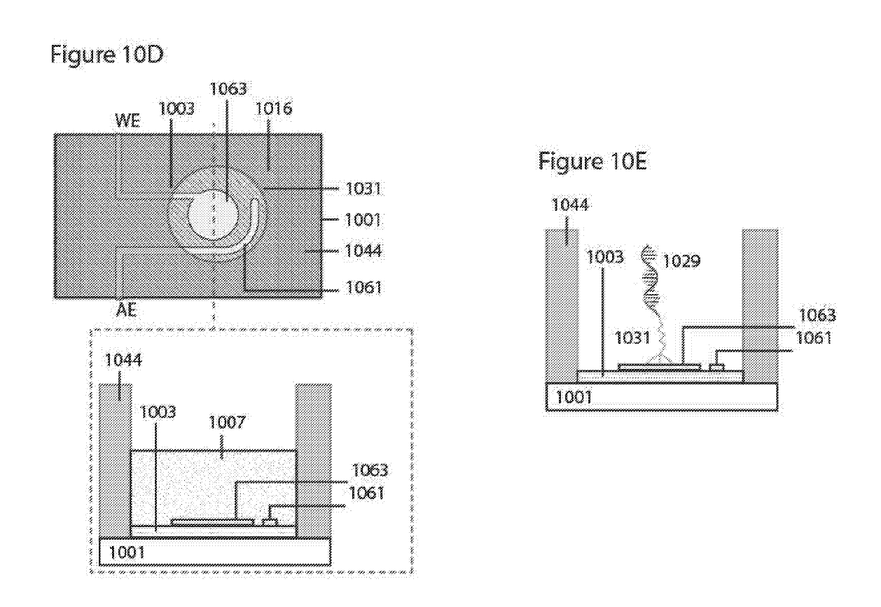

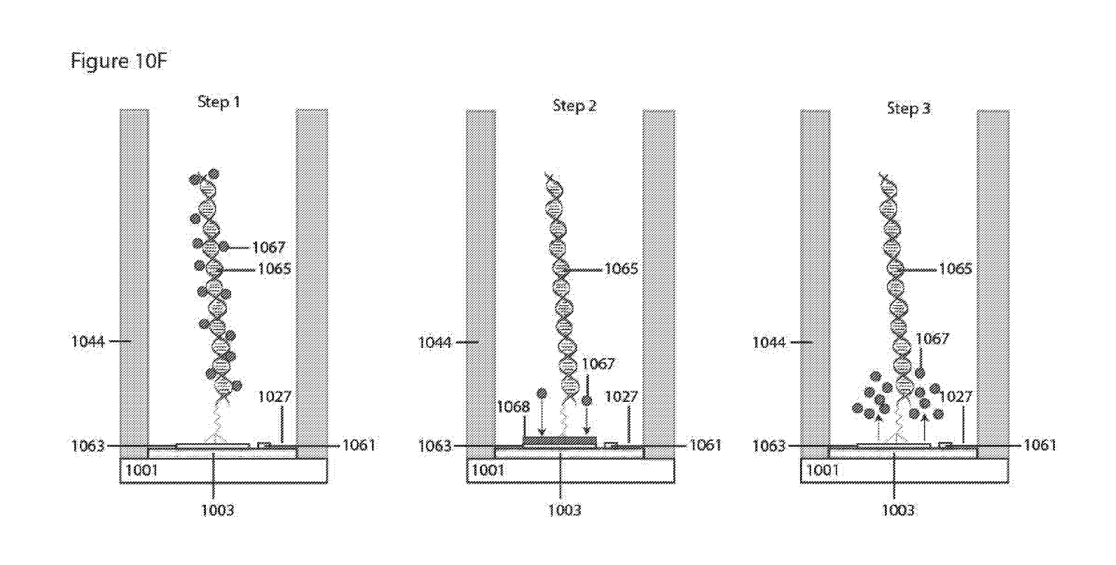

A variety of electrochemical detection techniques can be applied to detect amplified DNA products after both liquid-phase and solid-phase PCR utilizing reaction zones built with optically excited and/or electrically activated heat generation layers in the previous embodiments. Fabricating an electrochemical circuit on the substrate of the reaction zone(s) can enable a wide range of electrochemical detection techniques such as electrochemical impedance spectroscopy, cyclic voltammetry, stripping voltammetry, amperometry, potentiometry etc. The embodiments in FIGS. 10A-C illustrate a structure to build an electrochemical circuit can be built on a reaction zone configured with an electronically activated heat generation layer as well as a method to use the electrochemical circuit to detect amplified solid-phase PCR products. The embodiment in FIG. 10A shows one sample reaction zone from an array of reaction zones isolated by 3D pattern layer 1044 and configured with resistive heater 1003 and thermistor 1031 circuits fabricated on substrate 1001, similar to the embodiments in FIG. 8. Resistive heat circuit 1003 is used with thermistor circuit 1031 and/or a non-contact thermal sensor to perform PCR thermal cycling in the reaction zone(s). In this embodiment a working and auxiliary electrodes, 1063 and 1061 respectively, comprising an electrochemical circuit, are fabricated on top of passivation layer 1016 as illustrated in the cross sectional view of the reaction zone shown in the inset of FIG. 10A. The working and auxiliary electrodes of the electrochemical circuit may be fabricated by standard photolithography, deposition and etching techniques. The working and auxiliary electrodes may be made of inert metal such as gold, platinum, carbon etc. A third reference electrode, which can be shared by all of the electrochemical circuits fabricated on any or all of the reaction zones in the array of reaction zones, may be present in the cartridge and is not shown. The embodiment in FIG. 10B shows a cross sectional diagram of such a reaction zone configured for solid-phase PCR. At least one set of nucleic acid primers 1029 may be immobilized on the surface of the working electrode through interfacing chemistry 1031. FIG. 10C illustrates cross sectional diagrams of such a reaction zone after successful solid-phase PCR to demonstrate a stripping voltammetry method for electrochemical detection of the amplified DNA strand. In step 1, after successful solid-phase PCR which results in long double stranded amplicons 1065 extended on or near the surface of the working electrode, the reaction zone is exposed to a source of metal ions, for example, silver ions 1067 in silver nitrate solution. A wide variety of metal ions can be detected with anodic or cathodic stripping voltammetry techniques; silver is used as an example in this embodiment. Positively charged silver ions 1067 intercalate with and electrostatically attach to the negatively charged double stranded DNA amplicons 1065, causing a high concentration of silver ions to be bound near the surface of the reaction zone, proportional to the amount of amplicons present on or near the surface. After a wash step with buffer, in step 2 a ramping voltage is applied between the working and auxiliary electrodes, 1063 and 1061 respectively, causing the silver ions to undergo electrochemical deposition 1068 on the surface of the working electrode 1063. A layer of solid silver metal 1068 is deposited on the surface of the working electrode 1063 as a result. During deposition of the silver film 1068, the necessary electrons for this electrochemical process are provided by the electrochemical circuit, causing a distinct and gradual change in measured current. During step 3, the voltage applied to the electrochemical circuit is scanned in the reverse direction, causing the silver to release from the deposited silver film 1068 and dissolve back into solution as silver ions 1067. During this process, electrons are released by the silver and are captured by the electrochemical circuit, causing another distinct and gradual change in the measured current. Multiple cycles of stripping voltammetry can be performed to generate a reliable averaged peak current value as well as slope of change in current, which both indicate the amount of amplicons present on or near the surface of the reaction zone.

Electrochemical detection can also be carried out in a cartridge in which the reaction zones are configured with an optically excited heat generation layer. In fact, using an optically excited heat generation layer can reduce the complexity of the circuits which need to be fabricated on the reaction zones. The embodiments in FIGS. 10D-F illustrate a structure to build an electrochemical circuit can be built on a reaction zone configured with an optically excited heat generation layer as well as a method to use the electrochemical circuit to detect amplified solid-phase PCR products. The embodiment in FIG. 10D shows one sample reaction zone from an array of reaction zones isolated by 3D pattern layer 1044 and configured with an optically excited heat generation layer 1003, similar to the embodiments in FIG. 6. Optically excited heat generation layer 1003 is used with a non-contact thermal sensor to perform PCR thermal cycling in the reaction zone(s). In this embodiment a working and auxiliary electrodes, 1063 and 1061 respectively, comprising an electrochemical circuit, are fabricated on top of the heat generation layer 1003, as illustrated in the cross sectional view of the reaction zone shown in the inset of FIG. 10D. Alternatively, another substrate or passivation layer, for example, plastic or oxide, can be placed on top of the heat generation layer 1003, followed by fabrication of the electrochemical circuit on top of that substrate or passivation layer. The working and auxiliary electrodes of the electrochemical circuit may be fabricated by standard photolithography, deposition and etching techniques. The working and auxiliary electrodes may be made of inert metal such as gold, platinum, carbon etc. A third reference electrode, which can be shared by all of the electrochemical circuits fabricated on any or all of the reaction zones in the array of reaction zones, may be present in the cartridge and is not shown. The embodiment in FIG. 10E shows a cross sectional diagram of such a reaction zone configured for solid-phase PCR. At least one set of nucleic acid primers 1029 may be immobilized on the surface of the working electrode through interfacing chemistry 1031. The embodiment in FIG. 10F illustrates cross sectional diagrams of such a reaction zone after successful solid-phase PCR to demonstrate a stripping voltammetry method for electrochemical detection of the amplified DNA strand. In step 1, after successful solid-phase PCR which results in long double stranded amplicons 1065 extended on or near the surface of the working electrode, the reaction zone is exposed to a source of metal ions, for example, silver ions 1067 in silver nitrate solution. A wide variety of metal ions can be detected with anodic or cathodic stripping voltammetry techniques, silver is used as an example in this embodiment. Positively charged silver ions 1067 intercalate with and electrostatically attach to the negatively charged double stranded DNA amplicons 1065, causing a high concentration of silver ions to be bound near the surface of the reaction zone, proportional to the amount of amplicons present on or near the surface. After a wash step with buffer, in step 2 a ramping voltage is applied between the working and auxiliary electrodes, 1063 and 1061 respectively, causing the silver ions to undergo electrochemical deposition 1068 on the surface of the working electrode 1063. A layer of solid silver metal 1068 is deposited on the surface of the working electrode 1063 as a result. During deposition of the silver film 1068, the necessary electrons for this electrochemical process are provided by the electrochemical circuit, causing a distinct and gradual change in measured current. During step 3, the voltage applied to the electrochemical circuit is scanned in the reverse direction, causing the silver to release from the deposited silver film 1068 and dissolve back into solution as silver ions 1067. During this process, electrons are released by the silver and are captured by the electrochemical circuit, causing another distinct and gradual change in the measured current. Multiple cycles of stripping voltammetry can be performed to generate a reliable averaged peak current value as well as slope of change in current, which both indicate the amount of amplicons present on or near the surface of the reaction zone. Liquid-phase PCR products can also be detected by these electrochemical detection methods by simple immobilizing at least one set of complementary single stranded DNA probes on the surface of the working electrode to capture denatured amplified PCR products from solution, followed by the electrochemical detection protocol.

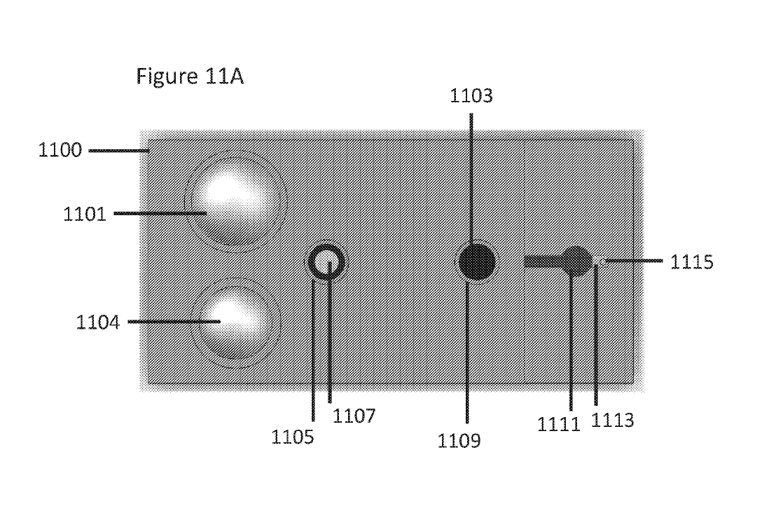

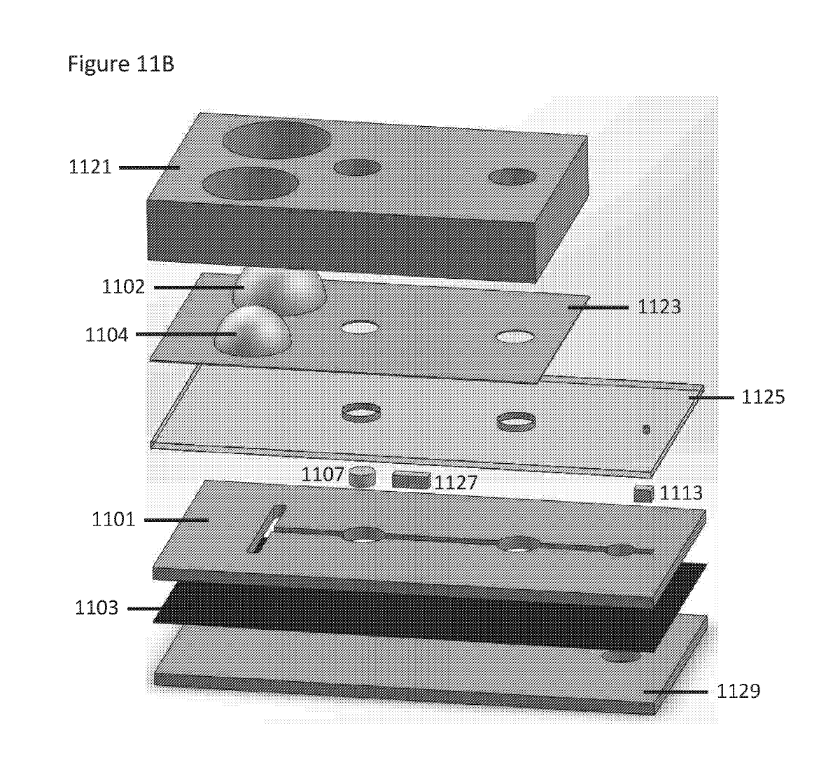

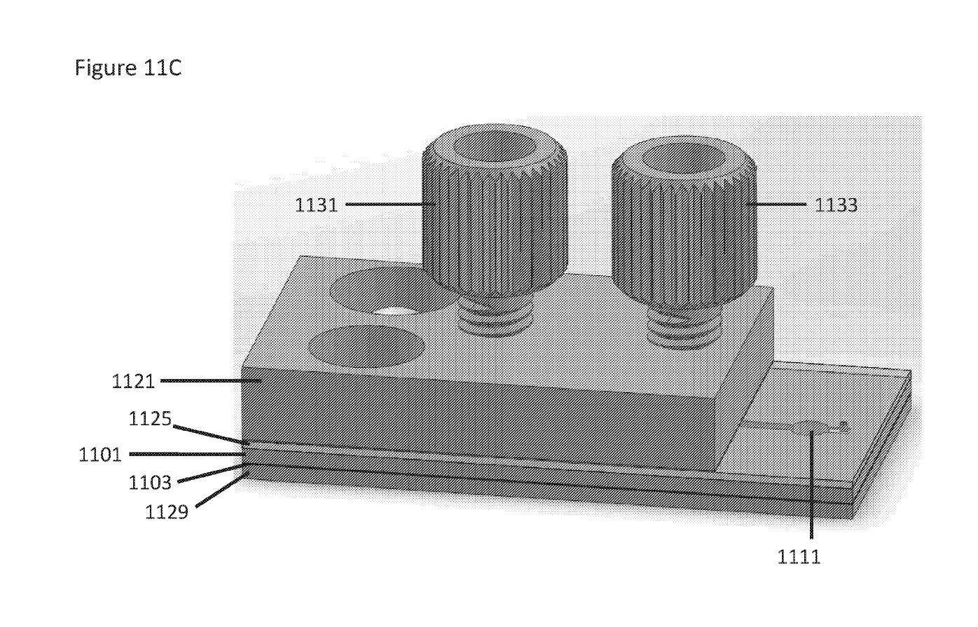

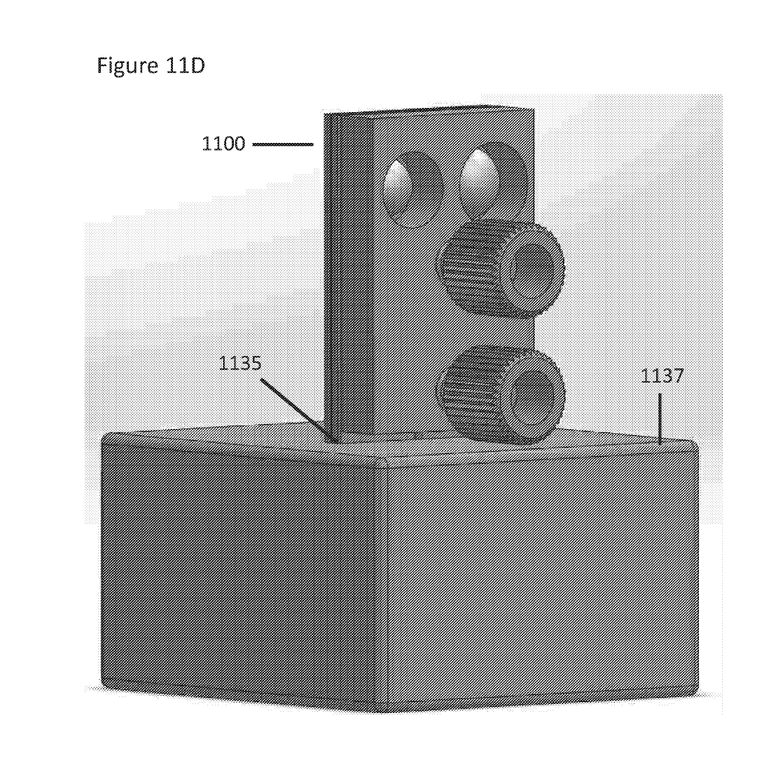

A prototype cartridge configured for liquid-phase PCR using an optically excited heat generation layer is shown in the embodiment in FIG. 11 as an example. The embodiment in FIG. 11A shows a top-view of the cartridge. Cartridge 1100 is comprised of layers of plastic adhered to each subsequent layer. The cartridge contains sealed chambers and channels for sample collection and manipulation as well as a final chamber where liquid-phase PCR occurs. The cartridge also contains dry on-board reagents for cell lysis and PCR in appropriate locations. Buffer is contained in blister pack 1102, while air is contained in blister pack 1104. Sample is collection in sample collection chamber 1105, where sponge 1107 serves as a sample collection sponge as well as a membrane onto which dry lysis reagents are pre-deposited. Valve chamber 1109 is an empty chamber used as a valve chamber into which a fitting (not shown) is inserted to block fluid flow. Heat generation layer 1103 can be seen in black and can be placed under the entire flow path or portions of the cartridge; in this embodiment, heat generation layer 1103 is a sheet of plastic which has been infused with absorptive black pigment. Liquid-phase PCR takes place in PCR chamber 1111. The right-end of the PCR chamber, which also marks the end of the channel structure, is fitted with a hydrophobic plug 1113, which resists fluid flow. A vent hole 1115 is placed in the plastic sealing film above hydrophobic plug 1113 to relieve back-pressure and facilitate fluid flow through the cartridge. The embodiment in FIG. 11B shows an exploded view of the cartridge to better illustrate its structure. The bottom of the cartridge is a sheet of plastic with a circular cutout on the right-hand side to allow light from an energy source in the reader (not shown) to expose the heat generation layer 1103 during PCR. Heat generation layer 1103 is a sheet of thin plastic infused with absorptive black pigment. The next layer is a channel definition layer 1101, which comprises a sheet of plastic through which the channel structure is cut out (throughout the depth of the plastic). Sponge 1107 is inserted into the sample collection chamber 1105. When the sample is inserted into the sample collection chamber, it hydrates the lysis reagents pre-dried onto sponge 1107. Hydrophilic filter 1127 is inserted into the portion of the channel which connects the sample collection chamber 1105 and valve chamber 1109. Filter 1127 serves to filter excess protein and cellular debris from the lysed sample and allow nucleic acids to flow through. Hydrophobic plug 1113 blocks flow of fluid while allowing air to pass, thereby facilitating fluid flow through the cartridge. Sealing layer 1125 is a sheet of plastic with appropriate cutouts to expose the sample collection and valve chambers as well as the vent hole to allow airflow. The next sheet of plastic 1123 holds the two blister packs containing buffer solution and air, 1102 and 1104 respectively. Finally a capping block 1121 provides protection for the underlying layers and depth for fitting components to seal and/or valve the exposed sample collection and valve chambers. The two small holes in capping block 1121 are threaded to match the threads of the fittings 1131 and 1133 shown in the next figure. The embodiment in FIG. 11C shows a side-view of the assembled cartridge. Fitting 1131 is screwed into the capping block 1121 by the user after inserting the sample into the sample collection chamber. The sample may be inserted directly or mixed with buffer in a vial, outside of the cartridge, before insertion into the cartridge. If the sample is mixed with buffer, the user inserts a specified amount of sample into the sample collection chamber with a disposable plastic pipette. Fitting 1131 is a plastic plug-type fitting which seals the top surface of the sample collection layer. Fitting 1133 is also screwed into capping block 1121 after insertion of the sample into the sample collection chamber. Fitting 1133 is different from fitting 1131 in that it contains a solid plug which extends beyond the bottom of the fitting and into the interior of the valve chamber such that it prevents fluid flow beyond the valve chamber and into PCR chamber 1111. This is necessary to contain the sample in the sample collection chamber for a specified duration of time to allow cell lysis to take place. After cell lysis has occurred, fitting 1133 is loosened by the user, allowing fluid to flow through the subsequent chambers. The blister pack containing buffer is depressed to release buffer solution into the channels of the cartridge. The buffer solution flows through to the sample collection chamber and mixes with the sample. It may also flow into the hydrophilic filter and continue into the PCR chamber. However, to ensure smooth flow, the blister pack containing air is depressed, thereby pushing the mixed buffer and sample through the hydrophilic filter and into the PCR chamber 1111. As shown in the embodiment in FIG. 11D, after the sample and buffer have been moved to the PCR chamber, the user inserts the cartridge 1100 into slot 1135 in reader 1137. Reader 1137 contains an energy source to optically excite the heat generation layer in the cartridge as well as a thermal sensor to monitor the temperature during PCR thermal cycling. Reader 1137 may also contain optics for real-time or end-point fluorescence detection of amplified products; fluorescence detection is facilitated by imaging the solution in the PCR chamber of the cartridge from the topside because sealing layer 1125 is transparent. Reader 1137 also contains a power source, control and processing circuitry as well as a communications circuit to communicate control and status commands as well as results with a host such as a smart phone or computer.

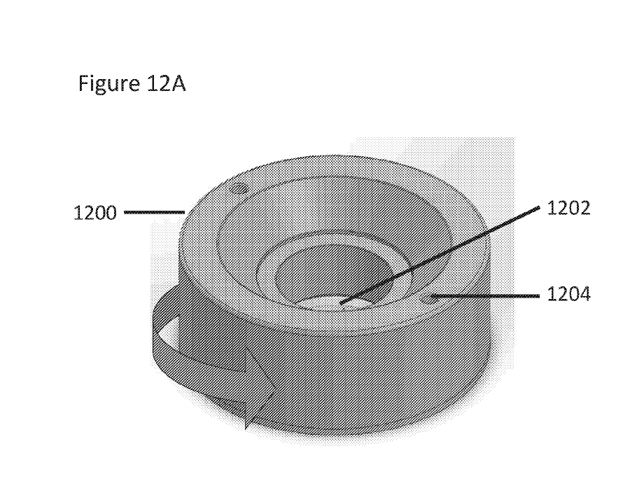

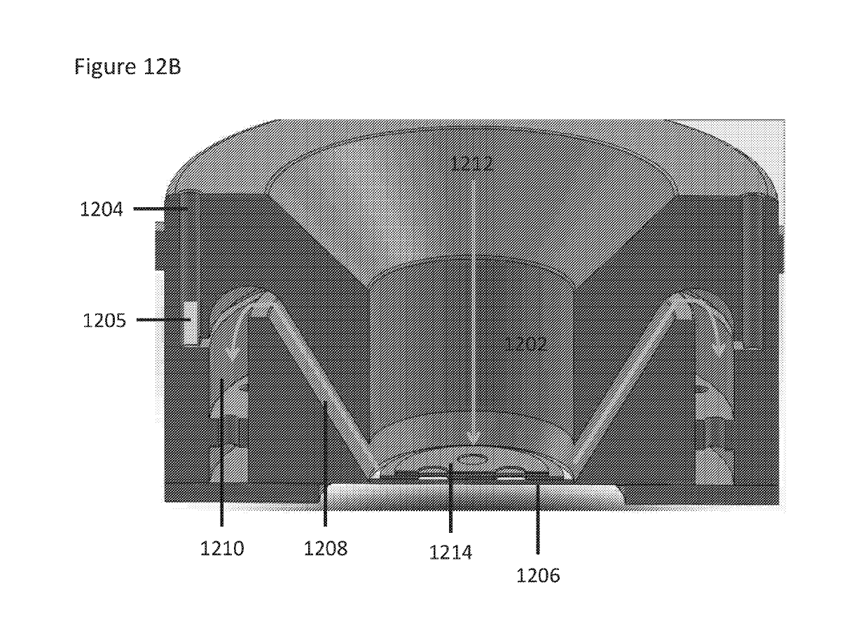

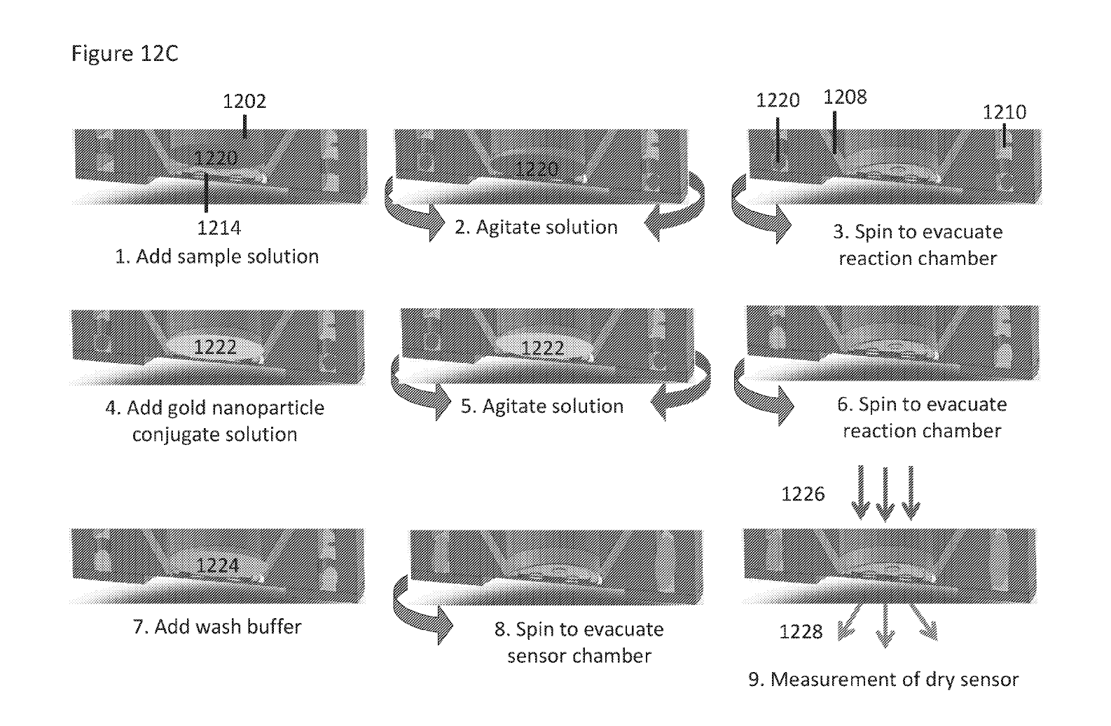

High sensitivity protein diagnostics is often performed by a multi-step and laborious process such as Enzyme Linked Immunoassay (ELISA). Complicated tests such as ELISA and other high performance assays are typically only performed in centralized labs due to the complexity of the assay and the equipment used to perform it. The embodiments in FIG. 12 shows a simple platform for automation of a multi-step diagnostic assay featuring automated sample processing, mixing and waste containment. The embodiment in FIG. 12A shows a side-view of the circular rotary cartridge 1200. The cartridge has a reaction chamber sunken 1202 down into the central volume of the cartridge. The top of the cartridge has two vent holes 1204. The blue arrow signified the circular motion of the cartridge during operation. The embodiment FIG. 12B shows a cross sectional view of the rotary cartridge 1200. Reaction chamber 1202 is a hollow cutout chamber in the central volume of the cartridge and holds sample and other fluids inserted into the chamber. The bottom surface of the reaction chamber is sealed with substrate 1206 which can comprise any assay components used in the protocol. In this embodiment, bottom substrate 1206 contains an assay chip 1214, which houses assay components and/or reagents needed for the assay. The chip can take a variety of formats, such as an electronic or plastic chip for different types of assays. The substrate and rotary cartridge can be adapted to allow for a variety of different types of assays. A hollow waste chamber 1210 surrounds the reaction chamber 1202 and serves as a repository for holding the different fluids used in the assay after they are used in the assay taking place on assay chip 1214 in the reaction chamber. Waste chamber 1210 and reaction chamber 1202 are separated by a hollow rising well 1208 having sloped side walls to allow fluid to flow from the reaction chamber and into the waste chamber. During the reaction phase, in which a solution in inserted into the reaction chamber during the assay, it remains in the reaction chamber due to the obstruction provided by the sloped walls of the hollow rising well 1208. Next, to remove reacted solution from the reaction chamber, the cartridge is rotated around its central axis by a motor in a reader or instrument such that the fluid is given energy to rise across the sloped side walls of the hollow rising well 1208 and fall into the waste chamber 1210, thereby clearing the previous solution from the reaction chamber, leaving it ready for the next step in the assay. Vent holes 1204 are blocked with hydrophobic plug 1205 such that air passes through the vent holes without leaking any solution. The waste is self-contained within the cartridge. In this manner, multiple solutions can be reacted with the surface of the assay chip 1214 in series without contamination from any other solutions used during the assay. The dimensions of the waste chamber 1210, reaction chamber 1202, substrate 1206 and assay chip 1214 can be configured based on the number of steps in an assay or total volume of solution used during an assay. The slope of the side walls of the hollow rising well 1208 can be designed to allow solution to flow above it and into the waste chamber only when the cartridge is rotated at a certain threshold speed. This allows for mixing or agitation of the solution in the reaction chamber during reaction steps at low or oscillating rotational speed.

The embodiment in FIG. 12C is a protocol to illustrate an example assay procedure using the rotary cartridge 1200 disclosed in the embodiments in FIGS. 12A and 12B. In the embodiment in FIG. 12C, only small cut-out cross sectional diagrams of the rotary cartridge are used for clarity and to highlight the bottom of the cartridge where the reactions take place in the reaction chamber. In step 1, a sample solution 1220, containing analytes of interest, is inserted into reaction chamber 1202 to allow it to react with assay chip 1214. In step 2, the rotary cartridge is rotated in an oscillating pattern to agitate and mix the sample solution 1220, allowing for more efficient reaction with the assay components on assay chip 1214. In step 3, the rotary cartridge is spun at a threshold speed which forces the sample solution 1220 to flow across the barrier of the hollow rising well 1208 and into the waste chamber 1210, thereby evacuating reaction chamber 1202. In step 4, a gold nanoparticle conjugate solution 1222 is inserted into the reaction chamber to react with assay components on the assay chip. In step 5, the rotary cartridge is rotated in an oscillating pattern to agitate and mix the gold nanoparticle conjugate solution 1222. In step 6, the rotary cartridge is spun at a threshold speed allowing the gold nanoparticle conjugate solution to evacuate the reaction chamber. In step 7, a wash buffer 1224 is inserted into the reaction chamber to remove unreacted sample, gold nanoparticle conjugate and other assay components from the assay chip before measurement. In step 8, the rotary cartridge is spun at a threshold speed allowing the waste buffer to evacuate the reaction chamber, leaving the assay chip ready for measurement. In the final step 9, an excitation source in the reader (not shown) excites the assay chip with energy 1226, causing emission of another energy 1228 from the assay chip which is then captured and analyzed by the reader (not shown). As illustrated in this example assay, a complex assay requiring multiple steps and solutions can be performed using the rotary cartridge in a straightforward manner without requiring additional equipment. The cartridge can be adapted to any assay protocol.

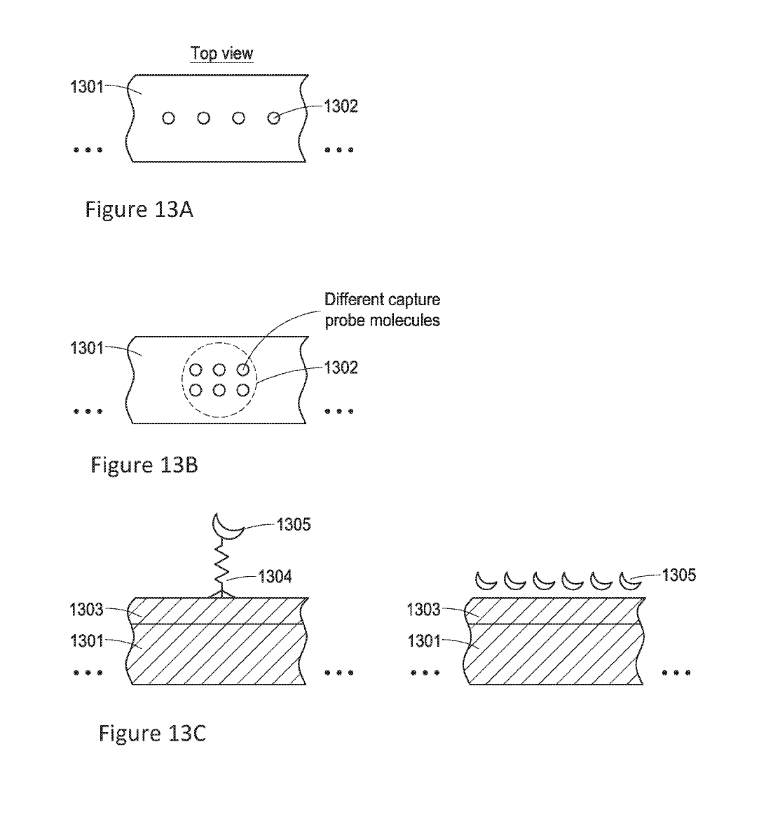

Some embodiments herein relate to devices and schemes for detection of biomarkers or analytes using nanoparticle tags for improving sensitivity, specificity, and performance. In some embodiments, as shown in FIG. 13A is disclosed a test region of an assay cartridge. The substrate 1301 contains one or more analyte capture regions 1302. The substrate 1301 can comprise a single material or a stack or layers of different materials. The substrate 1301 can comprise a material that does not absorb incident energy and therefore does not undergo self-heating, such as glass, reflective metal, transparent plastic or a semiconductor material. Another option is to coat the surface of a substrate 1301 with a reflective material, such as a reflective metal or dielectric mirror stack. The surface of capture regions 1302 comprise capture probe molecules for specific capture of protein, target DNA or RNA onto the surface. The capture regions 1302 are placed on substrate 1301 in discretely spaced regions, such that the incident energy source (for example, laser beam) is scanned across each capture region 1302 individually either by scanning the energy source or by physically moving the substrate 1301. The capture probe molecules can include a variety of capture probes such as a chemical molecule, antibody, single or double stranded DNA, and/or artificially created probes such as aptamer, DNAzyme, or synthetic capture molecules. In this manner, nanoparticle/analyte complexes from solution can be captured uniformly on the surface of the capture regions 1302 such that all or substantially all nanoparticles receive a uniform distribution of incident energy. Alternatively, as shown in the embodiment in FIG. 13B, multiple different capture probe molecules can be placed on a single capture region 1302, such that the incident energy source exposes all or substantially all of the capture probes in capture region 1302 at the same time. The spot size of the energy incident on the capture region or regions 1302 of the test region may be smaller than or larger than the total surface area that is functionalized with the capture probe molecules. As discussed in later embodiments, the density of capture probe molecules in the capture regions 1302 can be significantly increased by patterning the surface with 3D structures onto which capture probe molecules can be attached. Such 3D structures would increase the density of attached nanoparticles in a given area without causing non-uniform light exposure or shadowing, thereby increasing measured response or signal.

The capture probe molecules can be physically adsorbed to the surface of the capture regions in the test region or covalently attached via linker chemistry which binds the probes to the surface of the capture regions 1302. Covalent attachment of capture probe molecules is preferred as it would promote uniform and repeatable capture of analyte over the entire capture region 1302. Repeatability in surface density of captured analyte at any given concentration improves accuracy of results by narrowing distribution of thermal response between tests. FIG. 13C shows a cross sectional view illustrating a variety of probes covalently attached to the surface of the capture region. The substrate 1301 may be coated with an interfacial layer 1303. The interfacial layer 1303 can comprise more than one material or layer. For example, a highly reflective metal or dielectric stack can be coated on the substrate 1301, followed by a dielectric material or polymer for efficient chemical modification. This modification or interfacial layer would inhibit or prevent the substrate 1301 from self-heating upon exposure to incident energy. In fact, a reflective layer would reflect the incident energy which is not initially absorbed by nanoparticles back toward the nanoparticles for enhanced absorption and thermal response, thereby increasing sensitivity and improving signal-to-noise ratio at the same time. FIG. 13C, left side, shows embodiments in which the capture probe molecules are anchored to the surface of the capture region directly or to an interfacial layer 1303, by a linker molecule 1304 or by direct chemical modification of the surface to allow covalent attachment of capture probe molecules. Linker molecule 1304 may be attached to the surface of the capture region or, as illustrated in FIG. 13C, left side, to the interfacial layer 1303. The linker molecule 1304 is a bi-functional molecule, for example, silane or other chemical molecule, such that one terminal end of the molecule covalently attaches to the surface of the capture region or interfacial layer and another terminal end or ends contain a functional group for binding capture probe molecules. Alternatively or additionally, the probes may be physically adsorbed to the surface of the substrate 1301 as shown in FIG. 13C, right side. The capture probe molecules may be spot dried onto the capture regions of the test region by a micro-syringe or inkjet printing. Similar processes can be used to functionalize bare nanoparticles with linker molecules and/or capture probe molecules.

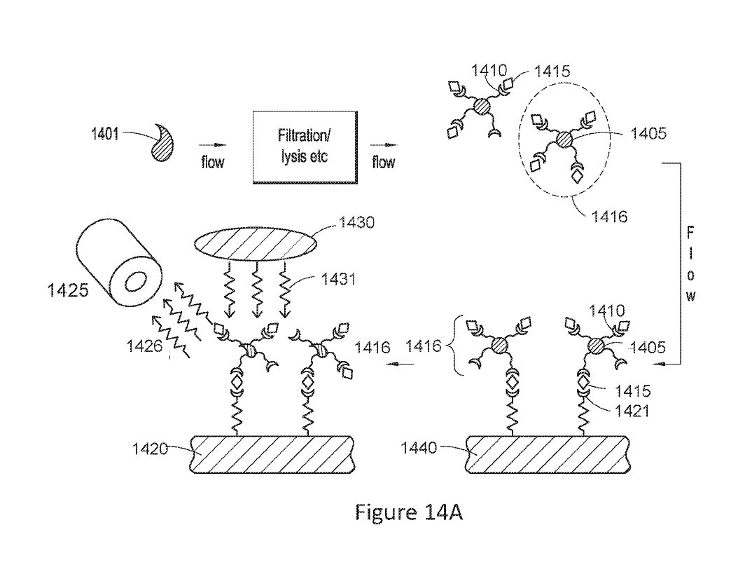

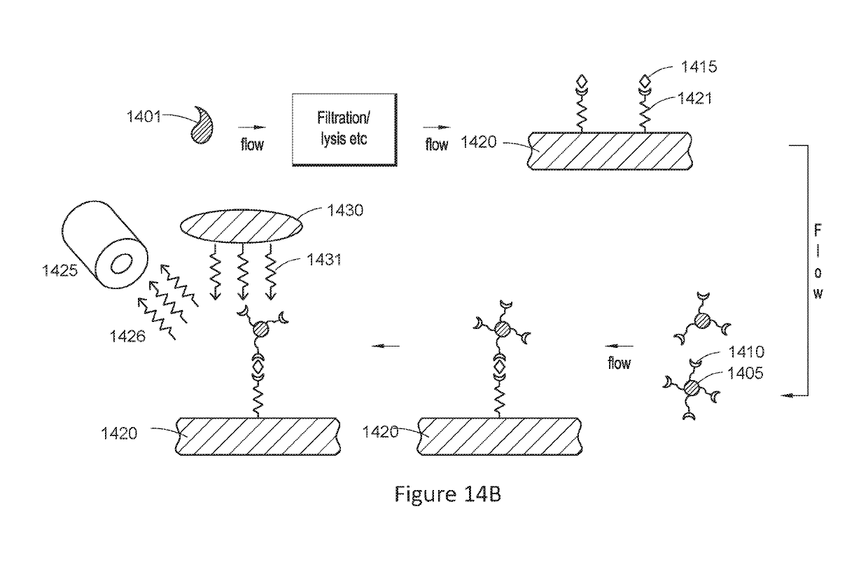

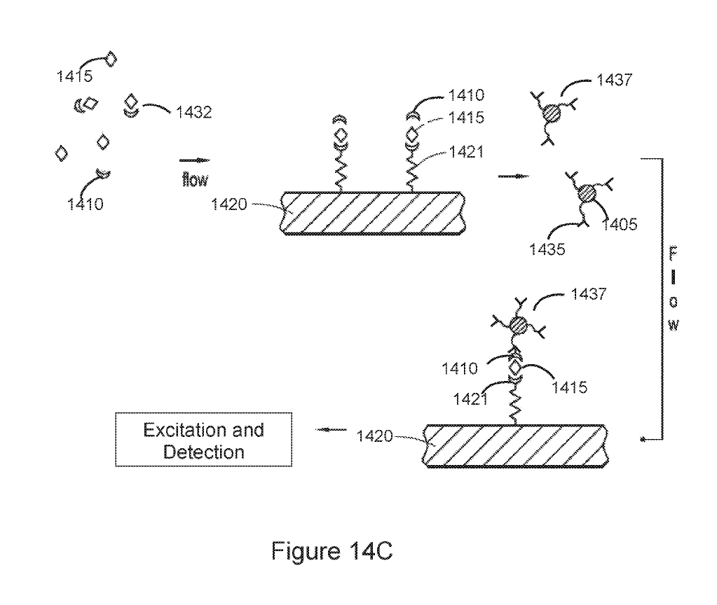

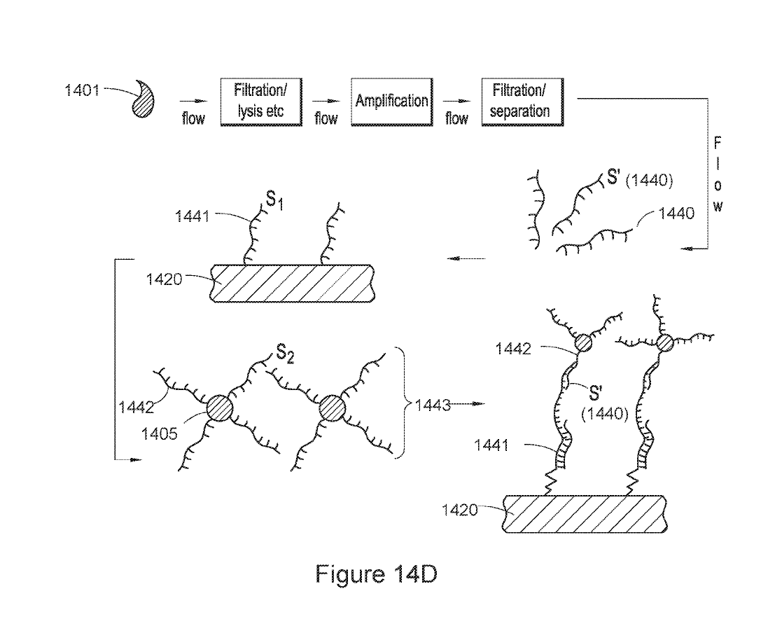

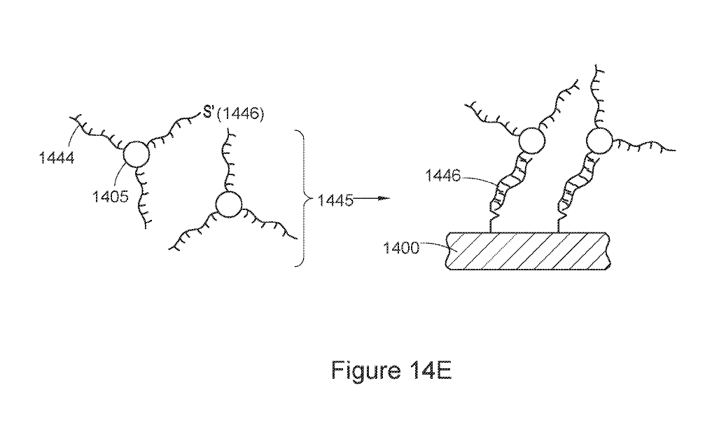

The embodiment in FIG. 14A illustrates one method to perform a nanoparticle assay with improved performance. Sample 1401 is flowed through a microfluidic assay cartridge or platform which is divided into separate areas for a variety of processes. Sample 1401 may undergo initial filtration, followed by cell lysis and additional filtration to remove cellular residue. Next, the sample is mixed with nanoparticles 1405 modified with capture probe molecules 1410 which capture the target analyte 1415, if it is present in the sample 1401. The nanoparticles 1405 may all be modified with the same capture probe molecule or different capture probe molecules for multiplexing. If multiplexing, the nanoparticles 1405 intended to capture different analytes may also be different in size and composition. The nanoparticles 405 may be stored on a solid surface of the assay cartridge or a membrane material deposited on top of the solid surface. The solution containing nanoparticle/analyte complexes 1416, comprising the nanoparticle 1405, the capture probe molecules and the captured analytes 1415, is then flowed toward the test region of the assay cartridge, where it binds to capture probes 1421 on the surface of the capture region 1420. The capture region 1420 can be a part of a microfluidic channel or region on the assay cartridge or it can be located on a separate substrate integrated into the assay cartridge. The substrate for the capture region 1420 can comprise a solid material such as plastic, metal, glass, or a semiconductor like silicon. Capture probes 1421 bind to and capture analyte 1415 on the nanoparticle/analyte complexes 1416 thereby anchoring the nanoparticles 1405 to the surface. One or more wash steps could be performed to help ensure all or substantially all of the non-specifically bound nanoparticles are washed away for increased specificity and accuracy. Next, an energy source 1430 (for example, laser beam) is incident onto the capture region(s) 1420 of the test region such that the captured nanoparticles/analyte complexes 1416 are exposed to the incident energy 1431. The nanoparticle/analyte complexes 1416 are likely immersed in fluid within the test region of the assay cartridge. The nanoparticles 1405 are selected such that they specifically absorb incident energy 1431. In response to incident light of resonant wavelength, selected nanoparticles undergo rapid heating and emit infrared energy 1426 which is then detected by a thermal detector 1425. Fluid could be removed from the test region prior to exposure to incident energy 1431 and/or measurement of thermal response. Removal of the fluid could be accomplished by flowing air over the test region to force the fluid out. Removal of the bulk of the fluid from the test region could enhance the measured thermal response, as less fluid would remain to absorb the infrared energy 1426 emitted by the captured nanoparticles 1405.

The embodiment shown in FIG. 14B illustrates another method for performing a nanoparticle assay. Sample 1401 undergoes the same optional filtration and lysis steps described in the previous embodiment. The sample 1401 is then immediately flowed to the test region where analyte 1415 in the sample attaches to the capture probe molecules 1421 on the surface of a capture region 1420 in the test region (FIG. 14B, upper right). One or more wash steps may help ensure non-specifically adsorbed analyte is washed away after specific capture of target analyte on the surface. Next, nanoparticles 1405 modified with capture probe molecules are flowed to the test region, where they bind to analyte 1415, which has already been captured on the surface of the capture region 1420 (FIG. 14B, bottom right and bottom middle). Again, one or more wash steps may help ensure non-specifically adsorbed nanoparticles are washed away after specific capture of nanoparticles 1405 on the surface. Next, energy source 1430 exposes the capture region 1420 and causes the captured nanoparticles 1405 to undergo heating, which is detected by thermal detector 1425. This method could improve performance by decreasing total test time. Due to a faster rate of diffusion of smaller molecules, the free analyte in solution is able to bind with the capture probes on the surface more efficiently and/or quickly than would a nanoparticle/analyte complex. Another benefit of this approach is that each nanoparticle 1405 binds only to a single analyte molecule captured on the surface, whereas in the previous embodiment of FIG. 13A, each nanoparticle 1405 could bind several analytes. A 1:1 ratio of nanoparticle-to-analyte is preferred, as multiple analytes bound to the same nanoparticle do not contribute to the measured signal and are therefore wasted. The ratio may be greater than 1:1 if non-specifically adsorbed nanoparticles are not removed. Conversely, the ratio may be less than 1:1 if a single nanoparticle has more than one analyte attached. The extent of this offset in the ratio depends on the surface modification process, assay conditions and procedure, as well as size and shape of nanoparticles used. The ratio may be, for example, between about 1:0.5 and about 1:1.5. Therefore, the method described in this embodiment could improve accuracy of results when correlating the measured thermal response to the estimated concentration of analyte in the sample 1401.

The embodiment shown in FIG. 14C, illustrates another method for performing a nanoparticle assay. After optional lysis and filtration steps, the sample, containing one or more analytes 1415, is mixed with capture probe molecules 1410, forming an analyte-probe complex 1432. The analyte-probe complexes 1432 are then flowed toward the test region where they are captured onto the surface of a capture region 1420 in the test region. The nanoparticles 1405 are modified with capture probe molecules 1435, which specifically bind to a certain site on the analyte 1410 (for example, protein A or protein A/G, which specifically binds to the Fc fragment of antibodies), are then flowed onto the capture region 1402. These nanoparticle/probe complexes 1437 then bind to the analyte-probe complexes 1432 on the surface of the capture region 1420, followed by excitation by energy source 1430 and detection with thermal detector 1425, for example, as described in the above embodiments. This method may be especially useful for simplified multiplexing. The same nanoparticle/probe complexes 1437 can be used for multiple different types of analytes, irrespective of the number of different target analytes in the sample, thereby reducing complexity and/or cost of materials. If detecting more than one analyte, multiple capture probe molecules, one type for each target analyte, are mixed with the sample prior to capture on the surface of the capture region(s).

One or more control regions can be added to the assay cartridge, each containing a known quantity of bound nanoparticles. These nanoparticles can be attached to the surface of the control region during manufacturing of the assay cartridge. A measurement of the thermal response of the control region(s) to incident energy would provide reference thermal readings for correlation of measured thermal signal(s) from the test region in estimating analyte concentration.

The embodiment in FIG. 14D describes a method for performing a nanoparticle assay, as it may be applied to detection of DNA. Sample 1401 undergoes cell lysis, filtration, DNA amplification (for example, polymerase chain reaction), and optional filtration and separation steps. Next, the DNA strands are separated such that only single stranded amplified target DNA 1440, with sequence S′, is allowed to flow toward the test region. The surface of the capture region 1420 is modified with single stranded DNA capture probe molecule 1441, with sequence S₁, which is at least partially complementary to amplified sequence S′ 1440. Nanoparticle/probe complexes 1443 comprise the nanoparticle 1405 and single stranded DNA capture probe 1442, with sequence S₂, which is also at least partially complementary to a separate portion of amplified sequence S′ 1440. After amplified sequence 1440 binds partially to the capture probe molecules 1441, the nanoparticle/probe complexes 1443 are flowed to the test region. The nanoparticle/probe complexes bind to the surface of the capture region via a portion of amplified sequence S′ 1440 as shown in FIG. 14D, bottom right. Subsequent excitation of the nanoparticles 1405 and measurement of thermal signal is similar or identical to the embodiments described above.

The embodiment described in FIG. 14E describes another method for performing a nanoparticle assay for DNA detection in which DNA amplification is performed on the nanoparticles 1405, with the resulting amplicons attached to the nanoparticles 1405. In this embodiment, the nanoparticle/probe complexes 1445 comprise the nanoparticle 1405 and capture probe strands 1444, which are the amplified strands with sequence S′. The complementary strand which would be attached to S′ on the nanoparticle after amplification is separated, leaving only S′ on the nanoparticle 1405. The surface of the capture region(s) 1420 in the test region is modified with a capture probe strand 1446, having sequence S, which is fully complementary to strand 1445, with sequence S′. Capture probe strand 1446 captures nanoparticle tag complexes 1445 followed by excitation and detection, for example, as described in the embodiments above.

In the above embodiments, the nanoparticles can be plasmonic or metallic nanoparticles such as gold or silver nanoparticles or nanoparticles made with materials which undergo joule heating such as graphene, polymer etc. The nanoparticles can comprise a single material or multiple different materials either mixed together or layered on top of one another in a core-shell structure. The layers can comprise plasmonic, metallic or dielectric materials (for example, gold nanoparticles with an oxide shell or vice versa). The nanoparticles can have a variety of shapes and sizes with sizes ranging from about 1 nanometer to about 1 millimeter. The shapes can be spheres, rods, cubes, cages, stars, urchins, sheets, tubes etc.

Furthermore, this thermal detection approach may also be used with paramagnetic, super-paramagnetic, or ferromagnetic nanoparticles (for example, magnetite etc.). In this case, the energy source is an alternating magnetic field. The detector can be a thermal detector or a magnetic field detector which would detect and calculate the difference in incident magnetic field energy and the additive or subtractive magnetic field energy imparted by the magnetic nanoparticles.

In one non-limiting example, the capture probe molecules are antibodies or aptamers towards one or more epitopes of the target protein, for example, cardiac troponin I protein (cTn I). The surface of the capture region is modified with anti-cTn I antibody 1, which captures one epitope of the target protein. The nanoparticles are 60 nm gold spheres modified with cTn I antibody 2, which captures another epitope of the target protein. The 60 nm gold nanoparticles are characterized to have a high absorption peak at a wavelength of about 532 nm. A green diode-pumped solid state laser (DPSS) with a center wavelength of 532 nm is used as the excitation source.

In another non-limiting example, the capture probe molecules are single stranded DNA, which is partially complementary to a sequence of DNA, or amplicon, amplified by a PCR reaction. In this example, the amplicon represents a sequence in the genome of the Ebola virus RNA, which is amplified in a reverse transcription PCR reaction. The surface of the capture region is modified with a sequence of single stranded DNA partially complementary to one terminal end of the amplicon. The nanoparticles are nanorods, with 10 nm diameter and 41 nm length, modified with another sequence of single stranded DNA that is complementary to the other terminal end of the amplicon. The 10 nm×41 nm nanorods are characterized to have a high absorption peak at a wavelength of about 808 nm. A near-infrared laser diode with center wavelength of 808 nm is used as the excitation source.

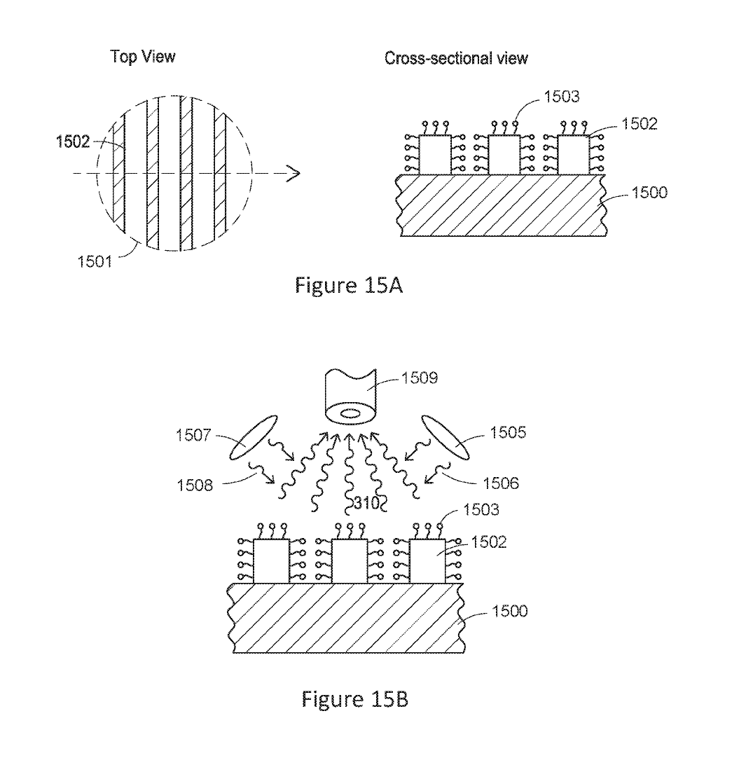

In order to increase the thermal signal for a given concentration of analyte, it may be advantageous to increase the density of nanoparticles within the spatial dimensions of the energy source (for example, laser beam) incident on the substrate. In the embodiment shown in FIG. 15A, a 3D patterned layer is added to the substrate as the top layer to which surface chemistry or linker molecules are functionalized. The top view shows a capture region 1501 in the test region with a three dimensional line grating patterned on its surface. The pattern comprises rectangular line structures 1502 protruding above the surface of the capture region 1501. The line grating pattern illustrated in FIG. 15A is one example. The 3D pattern can comprise pillars, pores or any other three dimensional structures which effectively increase the surface area available for specific capture of analyte and then nanoparticles. Alternatively, the 3D structures can be fabricated on a separate layer which is then added to the top of the substrate 1500 (cross-section view) or any additional layers which may already be on top of the substrate 1500. The 3D structures can also be fabricated directly from material of the substrate 1500. The dimensions of these 3D patterns can vary. The structures can be as small as one nanometer in any dimension, with nanoscale patterns generally being beneficial for highest surface area. The material which the 3D structures comprise preferably do not efficiently absorb the incident energy used to excite attached nanoparticles; suitable materials may include certain undyed polymers, photoresists, metals, metal oxides, semiconductors, or semiconductor oxides. The cross sectional view in FIG. 15A shows the 3D patterned lines 1502 on substrate 1500 of the capture region, with nanoparticles 1503 specifically attached to the surface. In this manner, a higher density of nanoparticles 1503 can be attached in a smaller area of the substrate 1500 which is exposed to the excitation energy source, effectively increasing the thermal response per unit area. This method also allows use of a smaller diameter laser beam as energy source, which increases the spatial power density of incident radiation energy, thereby allowing lower power laser sources to be utilized for excitation.

The embodiment in FIG. 15B shows one possible optimized implementation of the thermal detection method using a 3D patterned surface. Two energy sources 1505 and 1507, in this case laser diodes, are positioned at acute angles such that the incident laser beams 1506 and 1508 strike both sidewalls of the line grating 3D structures 1502, onto which nanoparticles 1503 are captured. A thermal detector 1509, which detects the infrared radiation 1510 generated by heated nanoparticles 1503, is positioned in the center such that its field of view encompasses the entirety of the capture region(s) 1502 and/or test region.

Infrared energy is readily absorbed by water, which is present in biological samples and is a constituent of many sample preparation procedures. However, absorption of the infrared energy emitted by captured nanoparticles 1503 can in variance in the measured thermal signal. The temperature of the water would increase as it absorbs infrared energy, albeit slowly given its high specific heat capacity. The loss of infrared energy to water would be especially problematic at low analyte concentrations, with fewer nanoparticles 1503 captured on the surface. Additionally, depending on the quantity of water within the test region, the time it would take for the temperature of the water to reach equilibrium within the test region would change the response time of the test. These issues would be exacerbated by any self-heating of the substrate in response to incident energy (for example, laser beam). These issues can be alleviated using a modified substrate and assay method described in the following embodiments.

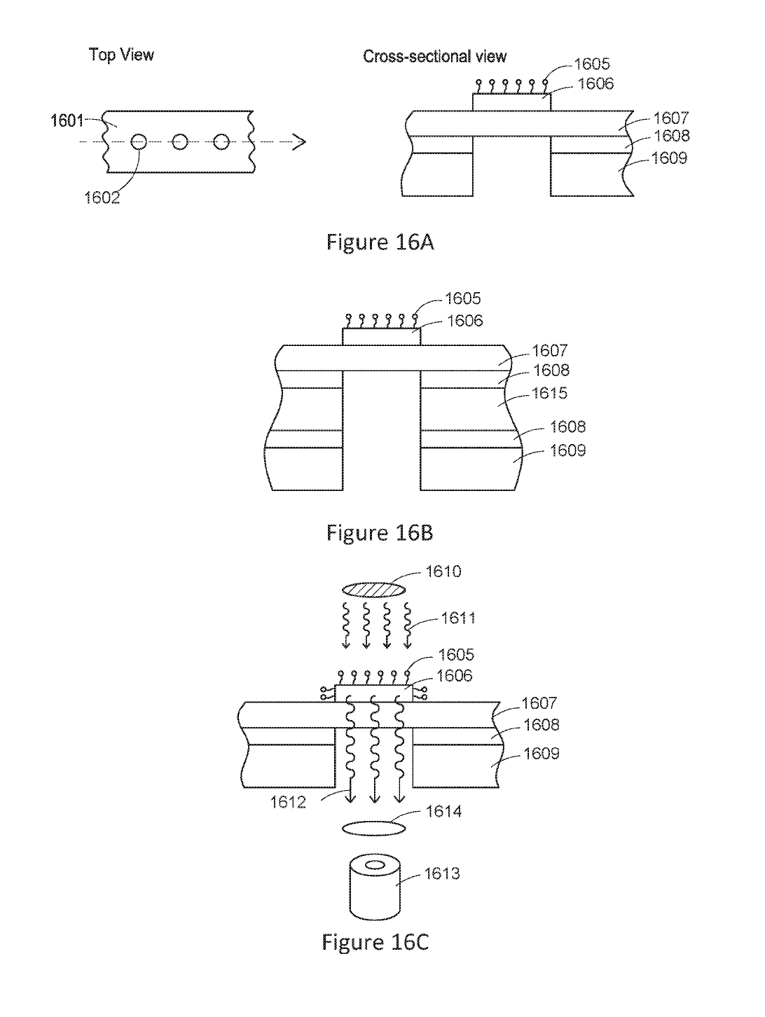

The embodiment shown in FIG. 16A describes a type of substrate 1601 designed to alleviate the aforementioned issues and/or improve performance. A microfluidic platform, including reaction chambers and enclosed channels, can be built around and on top the substrate 1601. The top view in FIG. 16A shows the test region on substrate 1601 comprising one or more capture regions 1602. The cross sectional view shows the construction of the substrate 1601, comprising of several layers of materials 1606, 1607, 1608, 1609. The base layer 1609 of the substrate 1601 comprises a layer of relatively thick and rigid material, such as polystyrene; this material can be plastic, metal, glass, fabric etc. The base layer 1609 provides sufficient rigidity to the substrate 1601, allowing for easier handling. The thickness of the material of the base layer 1609 can be between from about 1 micrometer to about several millimeters. Base layer 1609 is formed in such a manner as to have openings or holes in the location of the capture region(s) 1602. Layer 1608 comprises an adhesive material, such as liquid based adhesive or adhesive tape, which adheres surface layer 1607 to base layer 1609. Adhesive layer 1608 is also formed to have openings in the locations of the capture region(s) 1602. The openings in interfacial layer 1608 may be similar to or different in dimension compared to the openings in the base layer 1609. Layer 1608 may be between about several micrometers to several millimeters thick. Surface layer 1607 can comprise a low emissivity infrared transparent material that allows efficient transmission of infrared energy or a high emissivity material which allows efficient absorption of infrared energy. Surface layer 1607 preferably does not significantly absorb the incident energy (for example, laser beam) used to excite the nanoparticles. Surface layer 1607 may comprise a polymer, plastic, semiconductor, semiconductor oxide and/or metal oxide. In this embodiment, surface layer 1607 is low-density polyethylene (LDPE), a low emissivity plastic which transmits long wave infrared energy efficiently (>90%). It is preferred to use a material which is also thermally insulating and hydrophobic, such as the LDPE in this embodiment. A thermally insulting surface layer 1607 would inhibit or prevent thermal communication between individual capture regions within the test region, thereby increasing signal-to-noise ratio. A hydrophobic surface layer 1607 would allow sample or buffer to primarily or only be attracted to the comparatively hydrophilic capture regions. The thickness of surface layer 1607 can vary from about 1 micrometer to about several millimeters. It is preferred for the surface layer 1607 to be relatively thin, for example, relative to the base layer 1609, to inhibit or prevent significant attenuation of infrared energy. The surface of the LDPE surface layer 1607 is coated with an interfacial layer 1606, which may be patterned to only or primarily be present on the surface of individual capture regions 1602 for efficient surface functionalization. Interfacial layer 1606 may comprise a membrane material, polymer, semiconductor, semiconductor oxide, metal and/or metal oxide. In this embodiment, interfacial layer 1606 is patterned from SiO₂. This interfacial layer 1606 may also contain 3D structures to increase surface area, as described in previous embodiments. The thickness of interfacial layer 1606 can vary from about 1 nanometer to about several millimeters. However, it is preferred that the interfacial layer 1606 to be relatively thin, for example, relative to the base layer 1609, and transparent to infrared energy. Alternatively, surface layer 1607 may be used for surface chemistry and attachment of nanoparticles without interfacial layer 1606. Alternatively, surface layer 1607 may be used for some surface chemistry and attachment of nanoparticles and interfacial layer 1606 may be used for the same and/or other surface chemistry and attachment of nanoparticles.

The embodiment in FIG. 16B illustrates another implementation of the substrate 1601 described in the previous embodiment of FIG. 16A. In order to reduce or mitigate background signals due to accidental exposure of at least some of the adhesive layer 1608 and base layer 1609 to the incident energy (for example, laser beam), a reflecting layer 1615 may be added within the stack. This reflecting layer 1615 efficiently reflects the incident energy without causing the underlying materials of the lower adhesive layer 1608 and the base layer 1609 to heat up. Reflecting layer 1615 is sandwiched between adhesive layers 1608 such that its bottom surface is adhered to base layer 1609 and its top surface is adhered to surface layer 1607. There is also an opening in this reflective layer 1609 under the capture region(s) 1602 to facilitate infrared energy transmission. Reflecting layer 1615 may be a single material that is sufficiently reflective to incident energy, such as metal, semiconductor, or a dielectric mirror stack, or a combination of different materials, such as a plastic sheet coated with a reflective material.

The embodiment in FIG. 16C describes one possible configuration of the thermal detection system using the substrate(s) described in the previous embodiments. The substrate 1601, comprising layers 1606 through 1609 is shown with nanoparticles 1605 attached to the surface of the capture region. Energy source 1610, here a laser beam, exposes the capture region to incident energy 1611 to excite the attached nanoparticles 1605. The nanoparticles 1605 undergo rapid heating and emit omnidirectional infrared radiation 1612. While the nanoparticles 1605 may be surrounded by water, they are attached to within a few nanometers of the interfacial layer 1606. The absorption depth of infrared energy in water is at least a few dozen micrometers. As a result, the infrared energy 1612 is directly and efficiently transmitted through interfacial layer 1606 and surface layer 1607 without significant attenuation by the water surrounding the nanoparticles. A thermal detector 1613, placed behind the substrate and assay cartridge, measures this infrared energy 1612 emitted by the nanoparticles. A certain unabsorbed portion of incident energy 1611 could also be transmitted through to the thermal detector. To protect the detector 1613 from incident energy 1611 from energy source 1610, an automatic shutter 1614 may be installed in the reader device such that it blocks the incident energy 1611 while the nanoparticle tags are being excited. After a set period of time, coinciding with termination of the incident energy 1611, the shutter opens, exposing the detector to infrared energy 1612. The shutter 1614 can also be equipped with a semiconductor or thermocouple temperature measuring device to provide a measurement of the temperature of the shutter surface as a reference or calibration value for the thermal detector 1613. A frequency-specific measurement, taking advantage of improved response time, can be used to shorten total test time. During excitation the incident energy 1611 (for example, laser beam) can be toggled on and off at a certain frequency, again coinciding with closing and opening of the shutter 1614 such that the shutter is closed when the incident energy 1611 is exposing the capture region.

Using the substrate described in the previous embodiments of FIG. 16 along with the measurement of the infrared signal from the backside of the infrared-transparent substrate can enhance performance significantly by bypassing the water present on top of the nanoparticles. The infrared energy emitted by the nanoparticles is collected and measured without attenuation through the water in the sample or fluid solution surrounding the nanoparticles. Moreover, infrared energy emitted from the nanoparticles is directly and instantly measured, instead of the temperature of the water surrounding the nanoparticles. This method to perform the nanoparticle assay can therefore result in enhanced sensitivity, improved stability and/or improved response time.

Another type of substrate may be fabricated from a thin wafer of semiconductor material (for example, silicon) or a semi-rigid plastic (for example, Polyethylene Terephthalate or PET). A wafer compatible with well-established semiconductor manufacturing techniques can support uniform and consistent fabrication of high resolution 2D and/or 3D structures over large areas with nanoscale precision. Passive sensor strips, each containing one or more individually functionalized capture regions, can be fabricated to a high degree of precision. The passive strips can then be integrated into a microfluidic assay cartridge or any other detection platform.

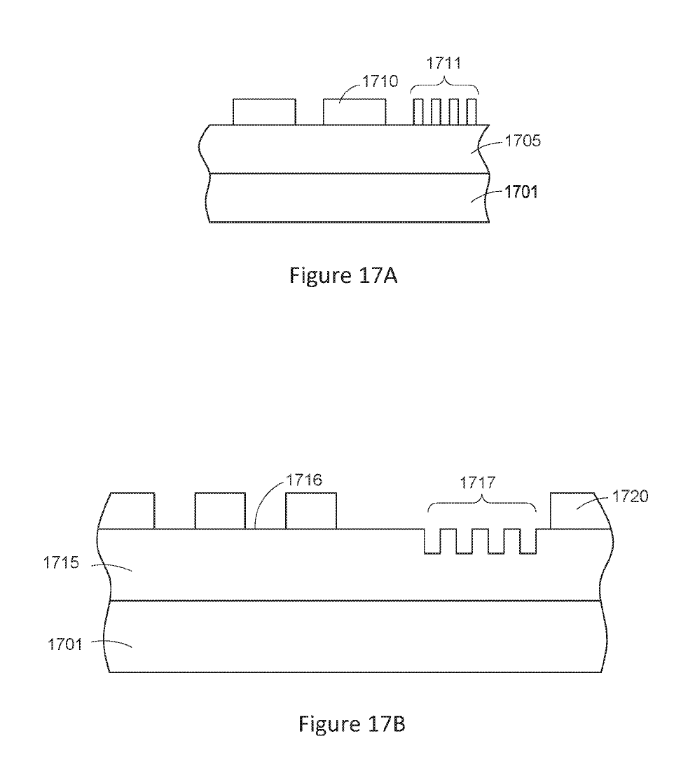

An embodiment describing such a substrate is illustrated in FIG. 17A. The substrate comprises a base layer 1701, which may be a semiconducting material (for example, silicon), glass, or a plastic wafer (for example, PET), that can be utilized as a wafer for subsequent processing and fabrication steps. Base layer 1701 may be between about several micrometers to several millimeters thick. Next, a thermal insulation layer 1705 comprising a material having low thermal conductivity is deposited on the base layer 1701 for thermal isolation from the substrate. This thermal insulation layer 1705 may not be necessary if the thermal conductivity of the base layer 1701 is sufficiently low (for example, if base layer comprises plastic). The thickness of thermal insulation layer 1705 may be between about 1 nanometer and about several millimeters. Next, capture regions, 1710 or 1711, are patterned on top of either the base layer 1701 and/or, as shown in FIG. 17A, thermal insulation layer 1705. The capture region 1710, 1711 may be fabricated from or coated with a suitable material which allows subsequent surface functionalization by silanes or other chemistry. In this embodiment, the capture regions 1710, 1711 are fabricated/patterned from a layer of SiO₂. The individual capture regions may be pattered and etched as 2D planar structures 1710 or 3D structures 1711. The capture regions 1710, 1711 are then functionalized with appropriate chemistry for bioconjugation of capture probe molecules.

The embodiment of FIG. 17B illustrates an alternate structure for the substrate. The substrate comprises a base layer 1701, which may be a semiconducting material (for example, silicon), glass, or a plastic wafer (for example, PET), that can be utilized as a wafer subsequent processing and fabrication steps. Next, layer 1715, which in this embodiment is SiO₂ either grown by oxidation of base layer 1701 made of silicon or deposited, is formed on or consuming part of the top of the base layer 1701. Layer 1715 can provide thermal isolation from the base layer 1701. Individual capture regions 1716 are defined by coating and patterning of a material 1720, which may be thermally insulating. In addition to providing thermal isolation, layer 1720 could also be hydrophobic and/or such that it cannot be functionalized by chemistry used to functionalize the capture regions, thereby further improving isolation between capture regions. Layer 1720 comprise (for example, may be patterned from) polymer or oxide. Layer 1720 can provide further thermal isolation from the base layer and/or thermal isolation between capture regions. As described in the previous embodiment of FIG. 17A, the individual capture regions can comprise 2D planar structures and/or patterned and etched to have a 3D structure. Layer 1720 may be patterned into 3D structures such as wells and channels that can define or at least partially define capture regions 1716, which may or may not include patterns in the layer 1715 such as capture regions 1717. Capture regions formed in the manner of the capture regions 1717 may be advantageous because they can be fabricated directly from (for example, etched into) the material of the layer 1715 (for example, comprising oxide) to have structures that can increase surface area and bound nanoparticle density (for example, 3D structures).

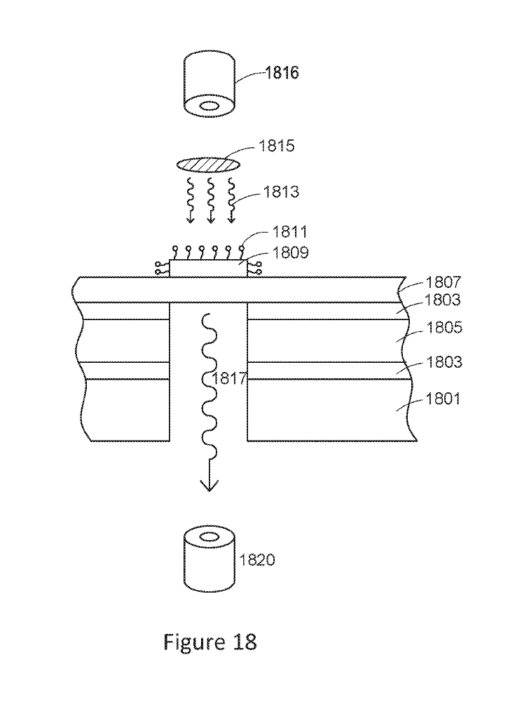

Plasmonic nanoparticles attached to a capture region on a substrate can also be detected by measuring light scattering if the substrate is transparent to scattered light. The substrates described in the embodiments of FIG. 16 can be used as such a transparent substrate. The embodiment of FIG. 18 illustrates the substrate and a method in which it can be utilized for this measurement. The substrate in FIG. 18 is constructed and used in the same manner described in the embodiment of FIG. 16. Scattered light 1817, rather than a thermal signal based on emitted infrared energy 1612, is used for measurement after capture of nanoparticles on the surface of the capture region. A light source 1816, such as a light emitting diode or laser diode, exposes the capture region 1809 with light of appropriate wavelength such that it causes the attached nanoparticles 1811 to scatter the incident light. As one non-limiting example, a 75 nm diameter spherical silver nanoparticles has a peak scattering at about 450 nm, so a light source having a center wavelength of about 450 nm is preferred. An optional filter 615 can further help ensure that the nanoparticles 1811 are only exposed with light of a particular wavelength or narrow range of wavelengths. Light (electromagnetic radiation) 1813 emitted from the source 1816 interacts with nanoparticles 1811 on the surface of capture region 1809. A certain amount of light 1813, in proportion to the concentration of attached nanoparticles 1811, is absorbed or scattered, causing a reduced intensity of light 1817 to be transmitted through the substrate. A light detector 1820, such as a photodiode, measures the intensity of transmitted light 1817. The difference between a reference reading, measured with light source 616 exposing capture region 1809 before attachment of nanoparticles 1811, and a test reading, measured after attachment of nanoparticles 1811, is calculated. The difference is proportional the density and number of nanoparticles attached and can therefore be correlated to the presence and concentration of analyte in the sample.

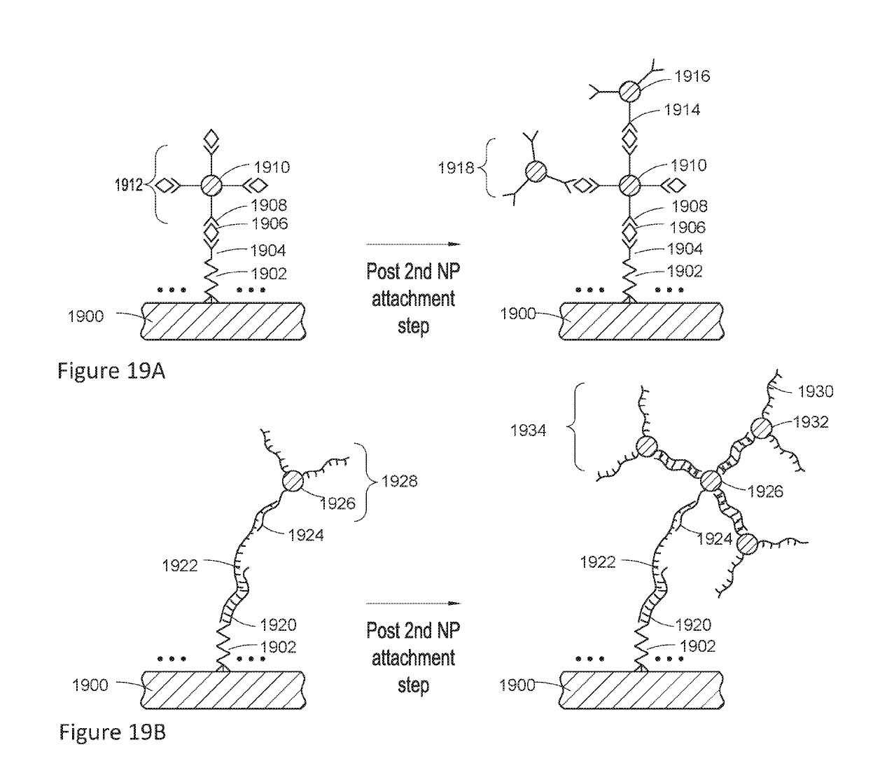

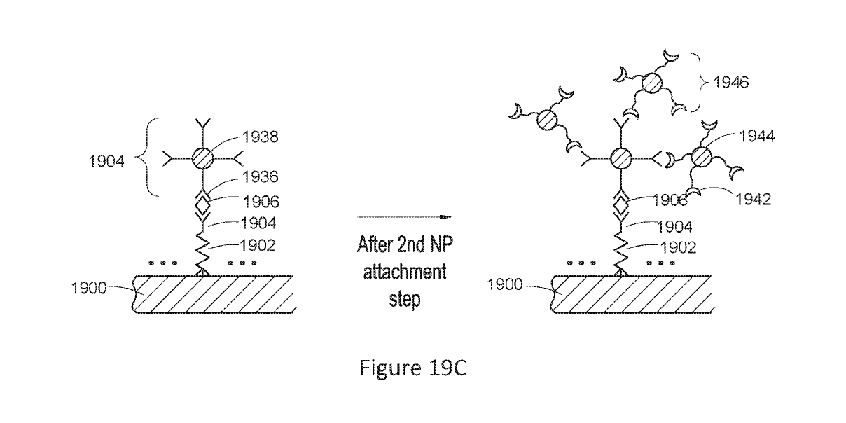

The use of a microfluidic cartridge for the nanoparticle assay allows methods to amplify of enhance the thermal signal. The nanoparticles which are specifically bound to the surface of the capture region via captured analyte generate thermal radiation in response to incident energy. By attaching additional nanoparticles to the first layer of nanoparticles, it is possible to amplify or enhance the thermal signal significantly. FIG. 19A illustrates one example with a molecular analyte, such as protein. The surface of the capture region 1900 is shown attached to capture probe molecules 1904 via linker molecules 1902. Capture probe molecules 1904, antibodies in this example, selectively bind nanoparticle-capture probe-analyte complex 1912, which includes the analyte 1906, capture probe molecule 1908 and the nanoparticle 1910. In this case, the sample containing analyte 1906 is reacted with the nanoparticle-capture probe complex prior to exposure to the test region containing the capture regions. After attachment, the nanoparticles 1910 captured on the surface will have at least some capture probe molecules 1908 which have analyte 706 bound to them and exposed; this exposed analyte can be used for additional capture steps. After selective attachment of the first set of nanoparticle-capture probe-analyte complex 1912 to the surface of the capture region 1900, a solution containing a second set of modified nanoparticles 1916 is dispensed to the test region. This solution contains nanoparticles 1916 that are modified with capture probe molecules 1914, which also selectively bind analyte 1906. The capture probe molecules 1914 modified on the surface of nanoparticles 1916 could be the same as capture probe molecules 1904 modified on the surface of the capture region 1900, could be different than capture probe molecules 1904 modified on the surface of the capture region 1900, or could be a combination that includes same and different capture probe molecules. This second set of nanoparticle-capture probe complexes 1918, which includes the capture probe molecule 1914 and the nanoparticle 1916, binds to exposed and unbound sites of analyte 1906, which is bound by one or a set of sites to nanoparticle-capture probe complex 1912. An optional wash step can be added after attachment of nanoparticle-capture probe complexes 1918, followed by exposure to incident energy and measurement of the thermal signal. This second nanoparticle attachment step yields additional nanoparticles attached to the surface of the capture region 1900, resulting in increased thermal signal upon exposure to incident energy. Additionally, additional analyte attached to the first set of nanoparticles is not wasted (for example, with a plurality of analyte binding to a single capture probe molecule emitting about as much thermal signal as if a single analyte had bound to that capture probe molecule) and contributes to the signal, which can increase the quantitative accuracy of the assay.

FIG. 19C illustrates a method of using secondary nanoparticle attachment in a system where the analyte is, for example, a protein. The surface of the capture region 1900 is shown attached to capture probe molecules 1904 via linker molecules 1902. Capture probe molecules 1904, antibodies in this example, selectively bind nanoparticle-capture probe-analyte complex 1940, which includes capture probe molecule 1936 and the nanoparticle 1938. In this case, the sample containing analyte 1906 is reacted with the capture region 1900 prior to exposure to the nanoparticle-capture probe complex 1940. After attachment, the nanoparticles 1938 captured on the surface will have at least some capture probe molecules 1936 which are exposed, the exposed capture probe molecules 1936 can be used for additional capture steps. After selective attachment of the first set of nanoparticle-capture probe-analyte complex 1940 to the surface of the capture region 1900, a solution containing a second set of modified nanoparticles 1946 is dispensed to the test region. This solution contains nanoparticles 1944 that are modified with capture probe molecules 1942, which, like the analyte 1906, selectively bind to capture probe molecules 1936 on the first set of nanoparticles 1938. The capture probe molecules 1942 can bind directly or indirectly (through a ligand or hapten) to capture probe molecules 1936. Capture probe molecules 1936 may be modified to comprise a ligand or hapten or molecule to which capture probe molecules 1942 can selectively bind but such that they do not interfere with the ability of capture probe molecule 1936 to bind to analyte 1906. An optional wash step can be added after attachment of nanoparticle-capture probe complexes 1946, followed by exposure to incident energy and measurement of the thermal signal.

FIG. 19B illustrates a method of secondary nanoparticle attachment used with an oligonucleotide analyte. The surface of the capture region 1900 is shown attached to capture probe molecules 1920 via linker molecules 1902. Capture probe molecules 1920, single stranded DNA in this example, selectively bind nanoparticle-capture probe-analyte complex 1928, which includes capture probe molecule 1924 and the nanoparticle 1926. Capture probe molecules 1920 and 1924 are at least partially complementary to either terminal of analyte strand 1922. In this example, a sample containing analyte strands 1922 have been reacted with the test region containing the capture region 1900 before dispensing the solution containing nanoparticle-capture probe complexes 1928. After attachment of the nanoparticle-capture probe complexes 1928, the nanoparticles 1926 captured on the surface will have at least some capture probe molecules 1924 which are not bound to analyte strands 1922. After selective attachment of the first set of nanoparticle-capture probe-analyte complexes 1928 to the surface of the capture region 1900, a solution containing a second set of modified nanoparticles is dispensed to the test region. This solution contains nanoparticles 1932 that are modified with capture probe molecules 1930 which are single stranded oligonucleotides that area at least partially complementary to capture probe molecules 1924. This second set of nanoparticle-capture probe complexes 1934 binds to exposed and unbound capture probe molecules 1924 on the first set of nanoparticle-capture probe complexes 1928. An optional wash step can be added after attachment of nanoparticle-capture probe complexes 1934, followed by exposure to incident energy and measurement of the thermal signal.

Utilizing a microelectronic or semiconductor device, which is sensitive to changes in temperature, to detect the heat generated by optically excited nanoparticles can be advantageous in the implementation of the nanoparticle assay, particularly for multiplexed tests. Multiple sensors can be arrayed on one chip for large scale multiplexing in a compact form factor, which can improve portability and/or reduce or minimize reagent usage. Performance may be improved by bringing the detector (sensing device) closer to the nanoparticles, which would be captured onto the surface of the semiconductor sensing device. Thermal energy generated by excited nanoparticles can be detected by conductive/convective heat transfer to the sensing element and/or by measurement of the infrared radiation emitted by excited nanoparticles.

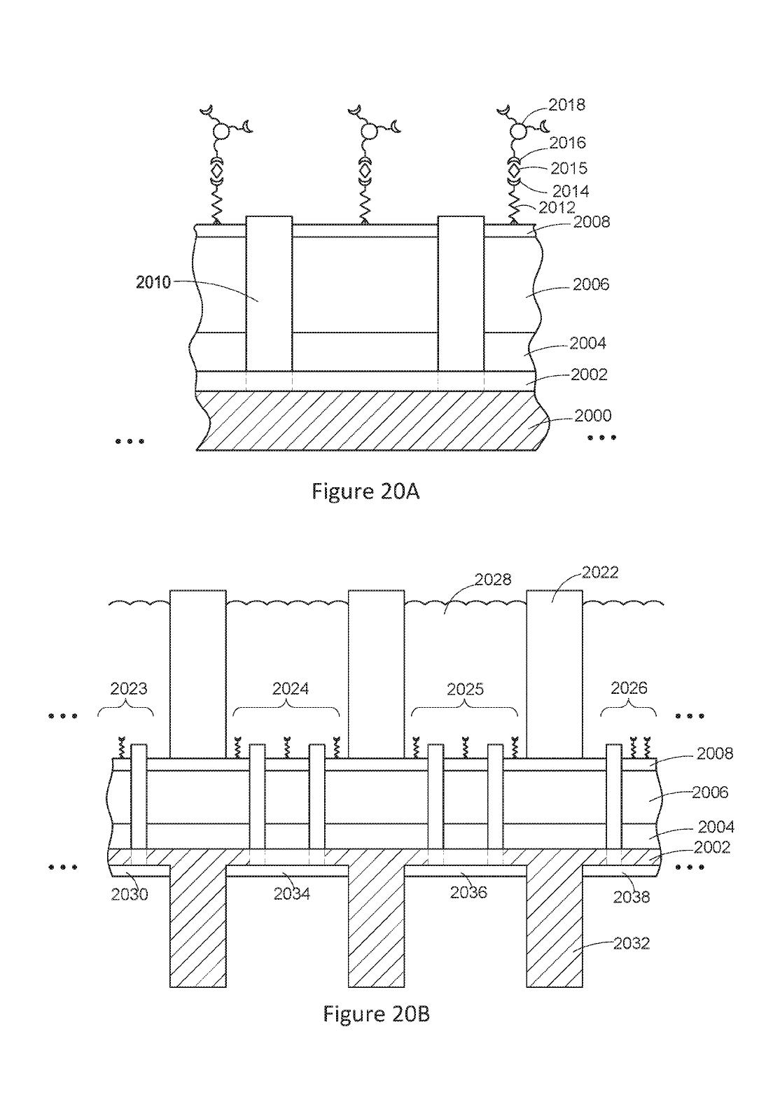

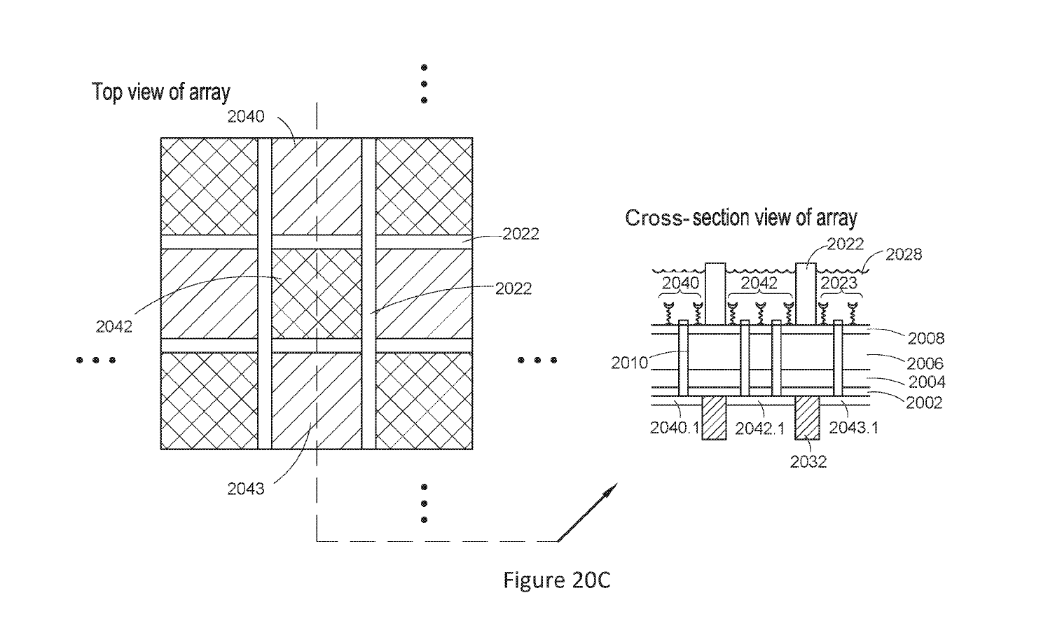

Heat generated by the nanoparticles may be detected by conductive/convective heat transfer to the temperature sensitive device via a thermally conductive contact. FIG. 20A shows an example of how a temperature sensitive device or element 2000 may be used in a nanoparticle assay. A temperature sensitive element 2000, which may comprise a p-n junction, p-i-n junction, diode, transistor, thermistor, resistance temperature detector (RTD), bolometer, etc., in which the current and/or voltage characteristics of the device undergo a predictable change with respect to temperature, may be utilized for transduction of heat emitted by excited nanoparticles 2018. Temperature sensitive device 2000 may be fabricated over a variety of substrates such as a silicon wafer, silicon-on-insulator wafer, glass, a plastic substrate, etc. Multiple such temperature sensitive devices 2000 may be fabricated in an array configuration and can comprise a test region of a sensor chip, forming multiple capture regions or pixels. Individual capture regions, which can be defined above each temperature sensitive device 2000 in the array (for example, the region shown in FIG. 20A), can be modified with different surface chemistry and/or biological capture probe molecules to capture multiple different analytes from the sample or from multiple samples. Layer 2002, which can comprise an electrical insulating material such as polymer or oxide, is an optional interfacial layer configured to electrically isolate the temperature sensitive device 2000. Interfacial layer 2002 may be as thin as several angstroms or nanometers. Layer 2004 comprises a material having low thermal conductivity, such as polymer, oxide, parylene, etc., configured to thermally isolate the substrate (not shown, under the temperature sensitive device 2000) as well as to provide thermal isolation between adjacent temperature sensitive devices 2000. Layer 2006 is an optional reflective layer configured to block incident energy which is used to excite the captured nanoparticles 2018. Layer 2006 may comprise a single or multiple layers of a reflective material (for example, metal), multiple layers of oxides forming a dielectric mirror, etc. The location of the thermally isolating layer 2004 and reflective layer 2006 may be interchanged (for example, layer 2004 may be over (for example, deposited over, deposited on top of) layer 2006). Layer 2008 is an interfacial layer configured to facilitate chemical and/or biological surface functionalization, for example, comprising a thin oxide layer for subsequent functionalization by silane. Layer 2008 may comprise a planar layer or may be patterned, for example, into a conformation such as a line gating or pillar pattern to increase the surface area of the capture region, as discussed herein. Material of the layer 2008 may be a capping material comprising the surface of a capture region. A capture region may be modified with linker molecules 2012 and capture probe molecules 2014 configured to capture a specific target analyte 2015 in a sample dispensed onto the test region. After capture of target analyte 2015, nanoparticles 2018, coupled to capture probe molecules 2016, are attached to the exposed captured analyte 2015, for example, via subsequent reaction and wash steps. After capture, nanoparticles 2018 are excited by incident energy, thereby producing heat. Reflective layer 2006 inhibits or prevents the incident excitation energy from reaching the temperature sensitive device 2000, which could be sensitive to light energy. Layer 2006 reflects the excitation energy back toward the nanoparticles 2018, thereby improving excitation efficiency. The heat generated by excited nanoparticles 2018 is conducted to the fluid surrounding the nanoparticles 2018 and is detected by temperature sensitive device 2000. As a result of the different properties of the multiple layers of materials between the surface of the capture region and the temperature sensitive device 2000, non-uniform conduction/convection of heat to the temperature sensitive device 2000 is possible. Vias 2010 comprising thermally conductive material may be patterned through the multiple layers 2004, 2006, 2008 and optionally 2002 to efficiently conduct the heat directly to the temperature sensitive device 2000. Small vias 2010 can result in a low thermal mass to enhance heat transfer efficiency. These vias 2010 may be fabricated in different shapes and configurations configured or optimized for heat transfer. One example is pillar shaped vias 2010 comprised of a thermally conductive material with low specific heat capacity, such as silver or copper. The upper surface of the vias 2010 can be exposed to the sample (as shown in FIG. 20A) or covered by the layer 2008. The fluid medium immediately surrounding the nanoparticles 2018 captured in the capture region heats up at a faster rate than the bulk of the fluid. Heat from this heated fluid is transferred to vias 2010 and subsequently to the temperature sensitive device 2000, which imparts a measurable change in the current or voltage characteristics of the temperature sensitive device 2000. Thermal isolation layer 2004 can reduce “thermal crosstalk” due to heat transfer between individual temperature sensitive devices 2000 in an array.

Thermal cross talk between adjacent devices through the fluid layer over the capture regions can be reduced or minimized by isolation between capture regions. Individual capture regions over an array of temperature sensitive devices 2000 may be separated and isolated from each other by patterned wells or channels comprising materials having low thermal conductivity such as oxides, polymer, parylene, etc. FIG. 20B illustrates an embodiment in which individual capture regions 2023, 2024, 2025, 2026 are isolated from each other by well or channel type features 2022. The wells/channels 2022 may separate the bulk of the fluid sample 2028 in each of the capture regions 2023, 2024, 2025, 2026 completely or partially. A possible advantage of this isolation of capture regions 2023, 2024, 2025, 2026 is that heat generated in the fluid 2028, and at the surface of the capture regions 2023, 2024, 2025, 2026, by the nanoparticles in each capture region 2023, 2024, 2025, 2026 is isolated from the fluid 2028 in adjacent capture regions. Thermal cross talk between adjacent temperature sensitive devices in the array (for example, 2030, 2034, 2036, 2038) may be reduced or minimized by fabricating trenches 2032 between individual temperature sensitive devices 2030, 2034, 2036, 2038. These trenches 2032 may be filled with material having low thermal conductivity (for example, polymer, oxide, parylene, etc.) or comprise air gaps.

The embodiment in FIG. 20C illustrates another method to reduce or minimize thermal cross talk between adjacent capture regions and/or temperature sensitive devices. Laterally adjacent capture regions, for example, 2040, 2042, 2043, are shown separated by well features 2022 (FIG. 20B). Laterally adjacent capture regions can be functionalized or used in a manner such that nanoparticles captured on either region are excited by different types of incident energy. For example, sections like 2040, 2043 (upward diagonal hatching) may be designed to capture 30 nm silver nanoparticles which are excitable by incident radiation with wavelength of 405 nm while sections like 2042 (outlined diamond hatching) may be designed to capture gold nanoparticles which are excitable by incident radiation with wavelength of 808 nm. Nanoparticles captured on capture regions directly and laterally adjacent to each other will not be excited at the same time, thereby reducing the possibility or at least the rate of thermal cross talk between adjacent capture regions.

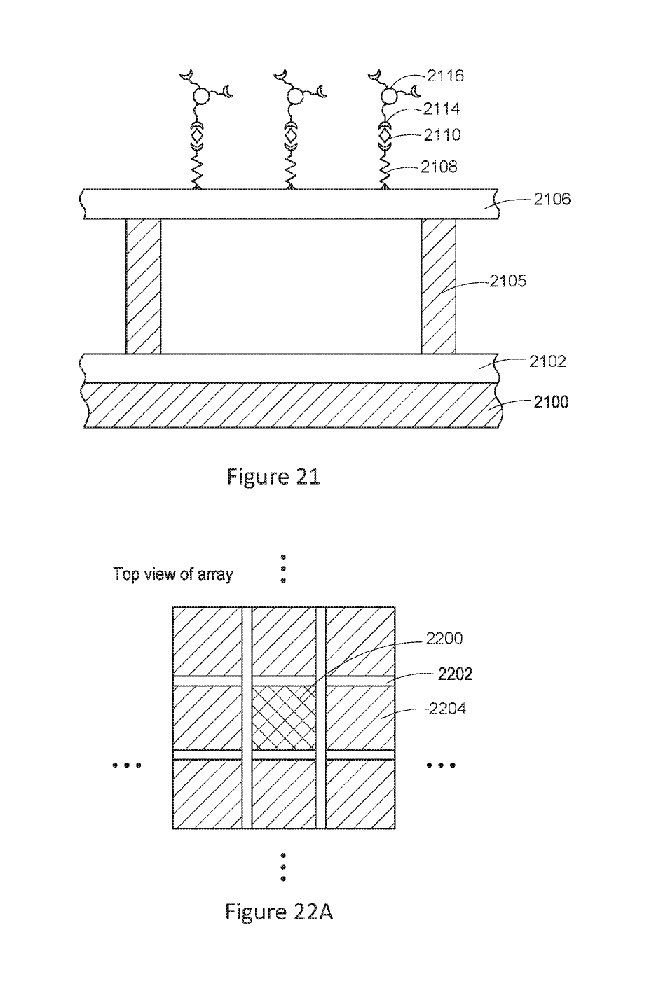

Another approach to detection of heat generated by nanoparticles is to utilize a microelectronic or semiconductor device which senses changes in temperature by non-contact means. The embodiment in FIG. 21 illustrates an example of a non-contact temperature and/or infrared sensitive element or device 2100 that can be used in a nanoparticle assay. Temperature/infrared sensitive element or device 2100, which is sensitive to the infrared radiation generated by heated (excited) nanoparticles, such as a bolometer or thermopile is used as the detector. Temperature/infrared sensitive device 2100 may be fabricated on a variety of substrates such as a silicon wafer, silicon-on-insulator wafer, glass, a plastic substrate, etc. Multiple such temperature/infrared sensitive devices 2100 may be fabricated in an array configuration and can comprise a test region of a sensor chip, forming multiple capture regions or pixels. Individual capture regions, which can be defined above each temperature/infrared sensitive device 2100 in the array (for example, the region shown in FIG. 21), can be modified with different surface chemistry and/or biological capture probe molecules to capture multiple different analytes from the sample or from multiple samples. Layer 2102 comprises a material having low thermal conductivity, such as polymer, oxide, parylene, etc. and is configured to thermally isolate the substrate (not shown, under the temperature/infrared sensitive device 2100) as well as to provide thermal isolation between adjacent temperature/infrared sensitive devices 2100. Layer 2104 is an optional reflective layer configured to block incident energy which is used to excite the captured nanoparticles 2116. Layer 2104 may comprise a single or multiple layers of a reflective material (for example, metal), multiple layers of oxides forming a dielectric mirror, etc. Layer 2104 is designed to reflect incident energy while allowing transmission of infrared radiation. The location of the thermally isolating layer 2102 and reflective layer 2104 may be interchanged (for example, layer 2102 may be over (for example, deposited over, deposited on top of) layer 2104). Layer 2106 is an interfacial layer configured to facilitate chemical and/or biological surface functionalization, for example, comprising a thin oxide layer for subsequent functionalization by silane. Layer 2106 may comprise a planar layer, for example, patterned into a conformation such as a line gating or pillar pattern to increase the surface area of the capture region, as discussed herein. A capture region may be modified with linker molecules 2108 and capture probe molecules 2110 configured to capture a specific target analyte 2112 in a sample dispensed onto the test region. After capture of target analyte 2112, nanoparticles 2116, coupled to capture probe molecules 2114, are attached to the exposed captured analyte 2112, for example, via subsequent reaction and wash steps. After capture, nanoparticles 2116 are excited by incident energy, thereby producing heat and infrared radiation. Reflective layer 2104 inhibits or prevents the incident excitation energy, but not infrared radiation, from reaching the temperature/infrared sensitive device 2100. Reflective layer 2104 may be configured to block a certain bandwidth with a center wavelength being substantially the same as the excitation laser's wavelength, and to allow other wavelengths such as infrared radiation pass there through (for example, by comprising a narrow-band dielectric mirror). Layer 2104 reflects the excitation energy back toward the nanoparticles 2116, thereby improving excitation efficiency. The infrared radiation generated by the captured nanoparticles 2116 heats up the temperature/infrared sensitive device 2100 and causes a measurable change in the current and/or voltage characteristics of the temperature/infrared sensitive device 2100. Using a temperature/infrared sensitive device 2100 as the detector could improve response time and performance because heat is indirectly measured before loss to the fluid and/or any conductive thermal mass. The thermal isolation layer 2102 can aid in isolating individual temperature/infrared sensitive devices 2100. Optional isolation features 2105 can be fabricated in between array elements to further isolate adjacent temperature/infrared sensitive devices in the array. Features 2105 can be configured as reflectors or absorbers, intended to reflect or absorb infrared energy. Features 2105 can comprise metal, polymer, oxide, etc., depending on their intended function. The device and capture region isolation methods disclosed herein (for example, with respect to FIGS. 20B and 20C) can also be applied to the temperature/infrared sensitive devices 2100.

The incident energy used to excite nanoparticles can comprise light energy. The light energy can have an optical wavelength which can be detected by silicon devices such as a diode. As discussed herein, the shape, intensity profile, spot size, and power of the incident energy beam can affect the signal measured by individual temperature/infrared sensitive devices in the test region. One exposure method exposes the entire test region with a beam of incident energy having a large spot size. Another exposure method uses a beam of incident energy having a small spot size, which is used to expose one capture region or plural capture regions at a time prior to measurement of the resulting signal. The incident energy beam may be differently calibrated based on the exposure method.

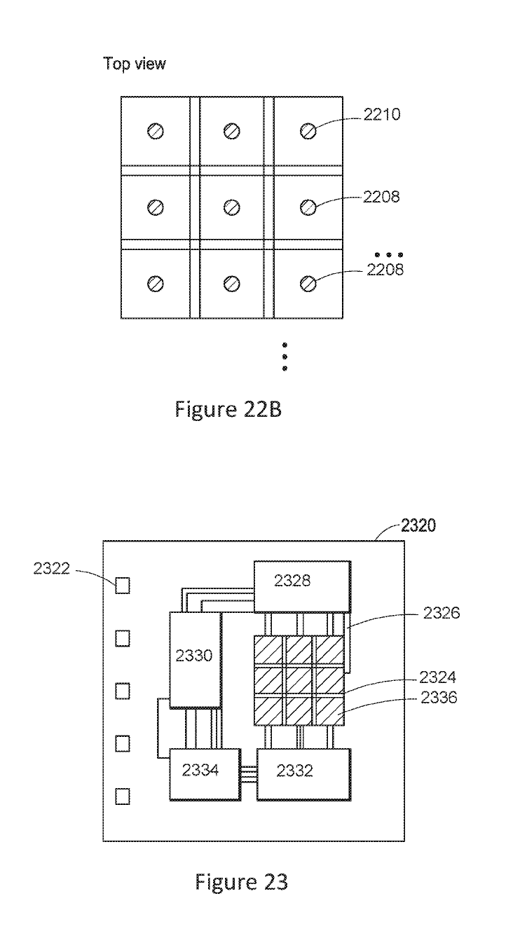

If a large spot size is used, the intensity profile can be calibrated with respect to position in the array. For example, given a laser beam with a Gaussian TEM00 intensity profile, the intensity at each point in the exposure area will vary in accordance with a Gaussian distribution. In order to align the laser beam and account for power fluctuations, light sensitive devices may be added to the array of temperature/infrared sensitive devices for calibration. FIG. 22A illustrates a test region of a sensor chip, showing individual capture regions in the array, with underlying temperature/infrared sensitive devices, as well as sparsely distributed light sensitive devices. Individual capture regions 2200 in the array can be encompassed with or laterally surrounded by adjacent light sensitive devices 2204. Wells 2202 can physically separate the light sensitive devices 2204 and capture regions 2200. Light sensitive devices 2204 such as photodiodes can be fabricated as pixels. The light sensitive devices 2208 can be built into each capture region 2210, as shown in FIG. 22B. Light sensitive devices 2208 can be fabricated in conjunction or independently of the temperature/infrared sensitive devices in capture regions 2210. The temperature/infrared sensitive devices may be utilized as light sensitive devices, for example, by patterning the reflective layer, 2006 (FIG. 20A), 2104 (FIG. 21), to have an opening at the location of 2208. The devices' current and/or voltage characteristics may change based on (for example, proportional to) the intensity of the incident energy. This measurement of the intensity of the incident energy beam can be used for closed-loop adjustment and calibration of the beam or adjustment of the measured thermal signal for a given intensity level. For example, if the array is illuminated with wide-area incident optical energy, with the beam having a large spot size, the non-uniform intensity profile (Gaussian or otherwise) can be estimated by measuring the intensity of the energy (with the light sensitive devices) at or around each temperature/infrared sensitive device in the array. The measured temperature (and rate of temperature change) of the nanoparticles captured on each device can be adjusted, by comparison to a calibration curve for correlation with analyte concentration, based on the calculated dose of incident excitation energy at or around each device in the array. This adjustment may provide proper correlation of measured results to concentration of analyte because the dose of excitation energy could vary significantly across the array, depending on the quality and intensity profile of the incident energy beam. This adjustment may also be applied to an example in which individual array elements are excited with an incident energy beam having a relatively smaller spot size, comparable to the size of each array element or several array elements. In this case, measurement of the intensity of incident energy can also be used to calculate dose in the manner described above. This estimated dose can then be applied to measured results for better correlation of results to estimating analyte concentration. This method can allow calibration of the beam due to variability in power output. For example, if intensity of the incident energy, as measured by the light sensitive devices in the array, does not match an expected or desired amount, the power of the device generating the incident energy (for example, laser diode) can be adjusted such that the output intensity is changed.

Another possible advantage of using microelectronic or semiconductor devices for on-chip capture and detection is the ability to integrate circuitry for signal processing. Circuitry for one, more, or all of device characterization, control, array addressing, amplification, signal processing, analog-to-digital conversion, input/output to a reader, etc. can be integrated onto a small sensor chip along with the test region containing the array of light sensitive devices and/or temperature/infrared sensitive devices. Additional circuitry can be fabricated along with the light sensitive devices and/or temperature/infrared sensitive devices in the test region in the same process using standard MOS manufacturing techniques, which can improve functionality and/or reduce cost. The embodiment in FIG. 23 shows one example layout of a tiny sensor chip with integrated processing circuitry. A sensor chip 2320 comprises integrated signal processing circuitry, the test region 2324, and contact pads 2322 configured to interface the chip 2320 with the reader. The test region 2324 is flanked by a variety of circuitry, for example, comprising control circuitry 2330 and a multiplexer 2328 configured to address individual sensor devices 2336 in the device array in test region 2324. The signal from sensor devices 2336 can be processed on-chip by circuitry 2332, which may include a de-multiplexer, amplification, and/or analog/digital conversion circuits. Input/output circuitry could facilitate communication with external circuitry and/or could contain wireless communication circuits, antennas, etc. Contact pads 2322 enable interface with external circuitry which may be in the electronic reader or adapter. Integrating circuitry into the tiny sensor chip 2320 can simplify the electronic reader/cartridge design and/or increase performance by reducing electrical noise.

Bolometer devices, typically used in infrared thermography applications due to their high sensitivity, are well suited as infrared sensitive devices 2100, for example, in the embodiment of FIG. 21. Microbolometers are made with MEMS fabrication techniques and typically have a floating pixel structure. In a typical MEMS microbolometer device, the active thermistor layer is suspended by bridging beam structures which provide a connection to the substrate for electrical characterization. The floating thermistor pixel exhibits high sensitivity to infrared radiation due to extreme isolation from the substrate (for example, vertically spaced from the substrate). The typical microbolometer chip is usually vacuum packaged and sealed for better isolation. A MEMS microbolometer device may be adapted for use as a detector of infrared radiation emitted by nanoparticles in a nanoparticle assay.

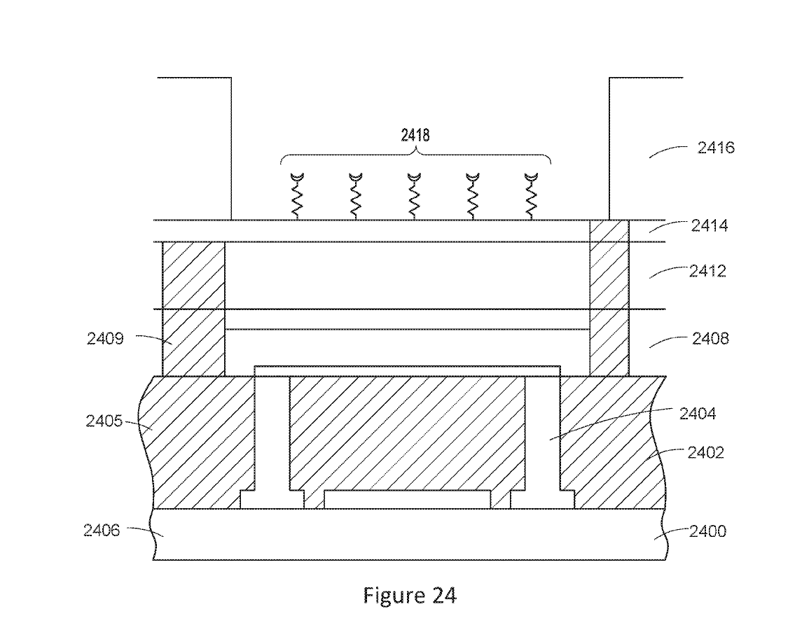

FIG. 24 illustrates several methods to fabricate a microbolometer sensor for compatibility with a nanoparticle assay. In FIG. 24, the region under the absorber/thermistor layer 2408 of the microbolometer is filled with a solid material (for example, material of the sealing layer 2402) for mechanical support and isolation from fluid. The substrate 2400 may comprise plastic, silicon, glass, etc., and the substrate 2400, or circuitry between the substrate 2400 and the bolometer, can contain addressing and readout circuitry for characterization of the bolometer device pixel. The studs or posts 2404, 2405 provide electrically conductive contacts connecting the thermistor layer 2408 to the substrate 2400 or circuitry over the substrate 2400. The studs 2404, 2405 maintain only a small area of contact to the thermistor layer 2408 to reduce or minimize heat dissipation to the substrate 2400 from the thermistor layer 2408 and/or heat transfer from the substrate 2400 to the thermistor layer 2408. Reflector 2406, illustrated as being proximate to the substrate 2400 and spaced from the thermistor layer 2408, is a reflective layer for any infrared radiation which may be transmitted through or emitted from the thermistor layer 2408. Additionally or alternatively, reflector layer 2406 may be proximate to the thermistor layer 2408 and spaced from the substrate 2400, for example, separated from the thermistor layer 2408 by a thin dielectric. In typical bolometer devices, the region under the floating thermistor layer is an air gap or vacuum. In the device shown in FIG. 24, the region under the thermistor layer 2408 comprises a mechanical support and sealing layer 2402, comprising thermally insulating material such as polymer, parylene, oxide, aerogel, etc., which provides sealed surface and/or support for subsequent fabrication steps. The thickness of layer 2402 may be calibrated such that the region between the thermistor layer 2408 and the reflective layer 2406 functions as a ¼ wavelength resonant cavity at a selected infrared wavelength. Absorber/thermistor layer 2408 is fabricated in the area under each capture region 2418 and comprises a temperature and/or infrared sensitive material, such as vanadium oxide, silicon, polysilicon, amorphous silicon, etc. An optional thermal insulation layer 2410, comprising thermally insulating material such as polymer, parylene, oxide, aerogel, etc. is formed over the thermistor layer 2408. Reflective layer 2412 is configured to reflect incident energy back toward the capture region 2418. Reflective layer 2412 may comprise a single material, a stack of different materials to make a dielectric mirror, etc. Reflective layer 2412 may be configured to block a certain bandwidth with a center wavelength being substantially the same as the excitation laser's wavelength, and to allow other wavelengths such as infrared energy pass there through (for example, by comprising a narrow-band dielectric mirror). Interface layer 2414 comprises an oxide or polymer to facilitate surface functionalization of the capture regions. Well or channel features 2416, comprising polymer, oxide, etc., may be fabricated to isolate individual capture regions and to inhibit or prevent cross talk between adjacent capture regions in the array. Optional isolation features 2409 may be patterned through thermal insulation layer 2410 and reflective layer 2412 to isolate the thermistor layers of adjacent microbolometer devices in the array from stray infrared radiation. Features 2409 can be configured as reflectors or absorbers, intended to reflect or absorb infrared energy. Features 2409 can comprise metal, polymer, oxide, etc., depending on their intended function. This structure allows for simultaneous multiplexed detection of nanoparticles attached to multiple capture regions in the array by the microbolometers fabricated under each array element. This structure could enhance detection sensitivity as the detector (microbolometer) is only a few hundred nanometers or few microns away from the nanoparticles. The nanoparticles attach to the capture region directly above the microbolometer device and efficiently emit infrared energy to the microbolometer upon excitation without significant absorption by the surrounding fluid.

While the inventions are susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims.

What is claimed is:

1. A cartridge for performing one or more nucleic acid amplification reactions comprising:
one or more reaction zones configured to receive reagents for performing said one or more nucleic acid amplification reactions involving a heating process; and
a heat generation layer in thermal communication with said one or more reaction zones, wherein said heat generation layer is configured to generate heat for at least one heating cycle via light provided by a light source.

2. The cartridge of claim 1, wherein the heat generation layer comprises pigments, dyes, pigmented or dyed plastic film or sheet, semiconductors, compound semiconductors, carbon nanotubes, fullerenes, graphene, oxides, graphene-oxide, metal-oxide, semiconductor-oxide, polymer, plastic, metal, metal-alloy, germanium, polyimide, glass, nanoparticles and/or microparticles, or a combination thereof.

3. The cartridge of any one of claims 1-2, wherein the heat generation layer comprises particles or beads.

4. The cartridge of any one of claims 1-3, wherein the one or more reaction zones are configured as one or more array of wells, holes, grooves, channels, or trench structures.

5. The cartridge of any one of claims 1-4, wherein the cartridge comprises a substrate comprising a material selected from the group consisting of semiconductor, metal, FR-4, polymer, plastic, epoxy, resin, glass, silicone, rubber, a track-etched membrane, and a combination thereof.

6. The cartridge of claim 5, wherein the substrate is transparent to light having a wavelength of about 400 nanometers to about 1 micrometer or any range therebetween.

7. The cartridge of claim 5, wherein the substrate is transparent to light having a wavelength of about 5 micrometers to about 13 micrometers or any range therebetween.

8. The cartridge of any one of claims 5-7, wherein the heat generation layer and the substrate combined have an emissivity of about 0.1 to about 1 in mid to far infrared range, or any range therebetween.

9. The cartridge of any one of claims 5-8, wherein the heat generation layer and the substrate combined have an emissivity of about 0.5 to about 1 in mid to far infrared range, or any range therebetween.

10. The cartridge of any one of claims 5-9, wherein the heat generation layer and the substrate combined have an emissivity of about 0.8 to about 1 in mid to far infrared range, or any range therebetween.

11. The cartridge of any one of claims 1-10, further comprising a thermal conduction layer in thermal communication with said heat generation layer.

12. A reader configured to receive the cartridge of any one of claims 1-11, said reader comprising:
a light source configured to provide light to said heat generation layer to generate heat for said heating process;
a detector configured to detect amplification products produced by said one or more nucleic acid amplification reactions; and
a thermal sensor which detects infrared light emitted from said heat generation layer or a circuit which communicates a signal indicative of temperature, said signal generated by a contact based temperature sensor in thermal communication with the heat generation layer in said cartridge.

13. The reader of claim 12, wherein the reader is configured to perform one or more heating cycles.

14. The reader of any one of claims 12-13, wherein the light source comprises a light emitting diode, an array of light emitting diodes, a laser diode, an array of laser diodes, a DPSS laser, an array of DPSS lasers, at least one focusing lens, at least one collimating lens, or a combination thereof.

15. The reader of any one of claims 12-14, wherein the detector is configured to detect fluorescence emitted by said amplification products.

16. The reader of any one of claims 12-15, wherein the thermal sensor comprises an infrared sensor.

17. The reader of claim 16, wherein the infrared sensor is a charge coupled device (CCD), complementary metal-oxide semiconductor device (CMOS), photovoltaic device, photodiode device, photoconductor device, thermopile device, bolometer device or a combination thereof.

18. The reader of any one of claims 12-17, wherein the infrared light is mid- to far-infrared.

19. The reader of any one of claims 12-18, wherein the infrared light has a wavelength that is from about 4 to about 16 micrometers, or any range there between.

20. The reader of any one of claims 12-19, wherein the infrared light has a wavelength that is from about 8 to about 14 micrometers, or any range there between.

21. The reader of any one of claims 12-15, wherein the thermal sensor comprises a contact temperature sensor.

22. The reader of claim 21, wherein the contact temperature sensor is a thermocouple, a resistance temperature detector, a thermistor, or a combination thereof.

23. The reader of any one of claims 21-22, wherein the contact temperature sensor is not in contact with a liquid in a sample in which an amplification reaction is being performed.

24. The reader of any one of claims 12-23, further comprising a cooling system configured to cool the one or more nucleic acid amplification reactions.

25. The reader of any one of claims 12-24, further comprising a detector configured to detect amplification products generated by said one or more nucleic acid amplification reactions.

26. The reader of claim 25 wherein said detector is configured to detect fluorescence emitted by said amplification products.

27. The reader of any one of claims 12-26, wherein said reader is a point of care reader.

28. A system comprising a cartridge of any one of claims 1-11 and a reader of any one of claims 12-27.

29. A method of performing one or more nucleic acid amplification reactions comprising at least one heating cycle, said method comprising receiving one or more samples at one or more reaction zones, generating heat at said one or more reaction zones by illuminating a heat generation layer in thermal communication with said one or more reaction zones, and performing nucleic acid amplification reactions on said one or more samples.

30. The method of claim 29, further comprising detecting a temperature of the heat generation layer.

31. The method of any one of claims 29-30, further comprising detecting amplification products.

32. The method of any one of claims 29-31, wherein the heat generation layer is illuminated by a light source comprising a light emitting diode, an array of light emitting diodes, a laser diode, an array of laser diodes, a DPSS laser, an array of DPSS lasers, at least one focusing lens, at least one collimating lens, or a combination thereof.

33. The method of claim 30, wherein detecting the temperature of the heat generation layer comprises detecting infrared light emitted from the heat generation layer using an infrared sensor comprising a charge coupled device (CCD), complementary metal-oxide semiconductor device (CMOS), photovoltaic device, photodiode device, photoconductor device, thermopile device, bolometer device or a combination thereof.

34. The method of any one of claim 30 or 33, wherein detecting the temperature of the heat generation layer comprises detecting the temperature using a contact temperature sensor comprising a thermocouple, a resistance temperature detector, a thermistor, or a combination thereof.

35. The method of any one of claims 29-34, wherein method comprises one or more heating cycles.

36. The method of any one of claims 29-35, wherein the method comprises one or more cooling cycles.

37. A point-of-care system for amplification and detection of nucleic acid molecules, comprising:
a test cartridge configured to perform nucleic acid amplification;
a reader device configured to detect nucleic acid amplification products; and
an energy source configured to heat a liquid sample in which a nucleic acid amplification process is performed.

38. The system of claim 37, wherein said energy source comprises a light source.

39. The system of any one of claims 37-38, wherein the test cartridge is configured to receive the liquid sample.

40. The system of any one of claims 37-39, wherein the test cartridge comprises one or more reaction zones, a substrate, or a heat generation layer.

41. The system of claim 40, wherein the one or more reaction zones is configured as one or more array of wells, holes, grooves, channels, or trench structures.

42. The system of claim 40, wherein the substrate is configured as a base for coatings, depositions, and/or fabrications of one or more 3D pattern layers, heat generation layers, thermal conduction layers, passivation layers, sample confinement layers, capping or encapsulation layers, or a combination thereof.

43. The system of any one of claims 40-42, wherein the substrate comprises a material selected from the group consisting of semiconductor, metal, FR-4, polymer, plastic, epoxy, resin, glass, silicone, rubber, a track-etched membrane, and a combination thereof.

44. The system of claim 43, wherein the substrate material is at least partially transparent in the wavelength range between 400 nanometers to 1 micrometer.

45. The system of claim 43, wherein the substrate material is at least partially transparent in the mid to long infrared spectrum wavelength in a range between 5 micrometers to 13 micrometers.

46. The system of claim 42, wherein the one or more 3D pattern layers are positioned under the heat generation layer and are configured to increase the surface area of the heat generation layer and/or increase the height of the heat generation layer so as to reduce the length that the reactants must diffuse to reach the heat generation layer.

47. The system of any one of claim 42 or 46, wherein the one or more 3D pattern layers comprises a material selected from the group consisting of polymer, plastic, silicone, rubber, glass, metal-oxide, semiconductor-oxide, and a combination thereof.

48. The system of any one of claim 42 or 46-47, wherein the one or more 3D pattern layers is a planar layer.

49. The system of any one of claim 42 or 46-48, wherein the one or more 3D pattern layers comprise patterned and/or deposited features and/or structures.

50. The system of claim 49, wherein the features and/or structures comprise one or more arrays of pillars, lines, line and space gratings, pyramids, triangles, trenches, spheres, or a combination thereof.

51. The system of any one of claims 49-50, wherein the features and/or structures are deposited and/or fabricated by photolithography, fused deposition modeling 3D printing, stereolithography 3D printing, selective laser sintering 3D printing, inkjet printing, molding, microarray printing/blotting/spotting, or a combination thereof.

52. The system of claim 47, wherein the material in the one or more 3D pattern layers is at least partially transparent in the wavelength range between 400 nanometers to 1 micrometer.

53. The system of claim 47, wherein the material in the one or more 3D pattern layers is at least partially transparent in the mid to long infrared spectrum wavelength in a range between 5 micrometers to 13 micrometers.

54. The system of claim 40, wherein the heat generation layer is positioned on top of a 3D pattern layer.

55. The system of any one of claim 40 or 54, wherein the heat generation layer is a light absorbing layer.

56. The system of claim 55, wherein the light absorbing layer is configured to absorb light energy input from the energy source and transform it into thermal energy.

57. The system of claim 56, wherein the thermal energy produced in the light absorbing layer is proportional to an amount of energy output from the energy source.

58. The system of any one of claims 55-57, wherein the light absorbing layer comprises a material selected from the group consisting of pigment, dye, semiconductor, compound semiconductor, carbon nanotubes, fullerenes, graphene, graphene-oxide, metal-oxide, semiconductor-oxide, polymer, plastic, metal, metal-alloy, and a combination thereof.

59. The system of any one of claims 55-58, wherein the light absorbing layer comprises germanium, polyimide, pigment or dye or a pigmented or dyed plastic film or sheet, nanoparticles and/or microparticles composed of metal, semiconductor, compound semiconductor, polymer, plastic, oxide, glass, or a combination thereof.

60. The system of claim 59, wherein the nanoparticles and/or microparticles are infused with and/or capped with light absorbing materials of claim 58.

61. The system of any one of claim 40 or 54-60, wherein the heat generation layer is a resistive heater layer.

62. The system of claim 61, wherein the resistive heater layer is configured as one or more traces and/or circuits to dissipate or absorb the voltage and/or current energy input from the energy source and transform it into thermal energy.

63. The system of claim 62, wherein thermal energy is produced in the one or more trace and/or circuit(s) of the resistive heater layer, which is proportional to the resistance of the trace(s) and/or circuit(s) of the resistive heater layer and the current flowing from the energy source and into the trace(s) and/or circuit(s) of the resistive heater layer.

64. The system of claim 63, wherein the trace(s) and/or circuit(s) of the resistive heater layer comprises semiconductor, compound semiconductor, carbon nanotubes, fullerenes, graphene, graphene-oxide, metal-oxide, semiconductor-oxide, metal, metal-alloy or any combination thereof.

65. The system of any one of claims 37-64, wherein the reader device is configured to receive the test cartridge.

66. The system of any one of claims 37-65, wherein the energy source comprises a light emitting diode, an array of light emitting diodes, a laser diode, an array of laser diodes, a DPSS laser, an array of DPSS lasers, at least one focusing lens, at least one collimating lens, or a combination thereof.

67. The system of any one of claims 37-66, further comprising one or more thermal sensors.

68. The system of claim 67, wherein the one or more thermal sensors comprise one or more non-contact infrared detectors.

69. The system of claim 68, wherein the infrared detector is a charge coupled device (CCD), complementary metal-oxide semiconductor device (CMOS), photovoltaic device, photodiode device, photoconductor device, thermopile device, bolometer device or any combination thereof.

70. The system of any one of claims 66-69, wherein the one or more thermal sensors are positioned to be under or above the liquid sample.

71. The system of any one of claims 66-70, wherein the one or more thermal sensors comprise one or more contact temperature sensors.

72. The system of claim 71, wherein the contact temperature sensor is a thermocouple, a resistance temperature detector, a thermistor, or a combination thereof.

73. The system of any one of claims 66-72, wherein the one or more thermal sensors are positioned to be in contact with the liquid sample.

74. The system of any one of claims 66-73, wherein the one or more thermal sensors are placed inside a sample confinement layer and in contact with the liquid sample.

75. The system of any one of claims 66-74, wherein the one or more thermal sensors are configured as a resistance temperature device or thermistor patterned and/or fabricated in close proximity to a heat generation layer.

76. The system of any one of claims 37-75, further comprising a thermal conduction layer.

77. The system of claim 76, wherein the thermal conduction layer is configured to facilitate heat transfer to the liquid sample and/or heat transfer from the liquid sample.

78. The system of any one of claims 76-77, wherein the thermal conduction layer comprises a material selected from the group consisting of metal, metal-alloy, semiconductor, compound semiconductor, graphene, carbon nanotubes, fullerenes, nanoparticles, microparticles, metal-oxide, semiconductor-oxide, and a combination thereof.

79. The system of any one of claims 37-78, further comprising a passivation layer.

80. The system of claim 79, wherein the passivation layer is configured to form an interface between the liquid sample and the energy source.

81. The system of any one of claims 79-80, wherein the passivation layer comprises a material selected from the group consisting of metal-oxide, semiconductor-oxide, glass, photoresist, plastic, polymer, semiconductor, metal, metal-alloy, and a combination thereof.

82. The system of any one of claims 79-81, wherein the passivation layer comprises a surface, wherein the surface of the passivation layer is coated or modified with chemical molecules, silane, protein, nucleic acids, or a combination thereof.

83. The system of any one of claims 37-82, further comprising a liquid sample comprising DNA, polymerase, DNase inhibitor, forward primer sequence strands, reverse primer sequence strands, free unlabeled nucleotides, free nucleotides labeled with one or more molecules, water, buffer salts, metal ions, or a combination thereof.

84. The system of any one of claims 37-82, further comprising a liquid sample comprising RNA or mRNA, reverse transcriptase, polymerase, RNase inhibitor, forward primer sequence strands, reverse primer sequence strands, free unlabeled nucleotides, free nucleotides labeled with one or more molecules, water, buffer salts, metal ions, or a combination thereof.

85. The system of any one of claims 37-84, further comprising a sample confinement layer, wherein the sample confinement layer comprises a well, hole, groove, or trench structure.

86. The system of claim 85, wherein the well, hole, groove or trench structure is fabricated from metal-oxide, semiconductor-oxide, metal, metal-alloy, glass, plastic, polymer, photoresist, silicone, rubber, or a combination thereof.

87. The system of any one of claims 85-86, wherein the sample confinement layer is coated with thermally conductive material selected from the group consisting of metal, metal-alloy, semiconductor, compound semiconductor, graphene, fullerenes, carbon nanotubes, nanoparticles, microparticles, and a combination thereof.

88. The system of claim 87, wherein the thermally conductive material is coated with a passivating material selected from the group consisting of metal-oxide, semiconductor-oxide, glass, photoresist, plastic, polymer, semiconductor, metal, metal-alloy, and a combination thereof.

89. The system of any one of claims 37-88, further comprising a capping or encapsulation layer, wherein the capping or encapsulation layer is configured to prevent evaporation of the liquid sample.

90. The system of claim 89, wherein the capping or encapsulation layer comprises a film of oil, plastic, or glass.

91. The system of any one of claims 37-90, further comprising a supplementary heating device, wherein the supplementary heating device is a thermoelectric device, a heat block, a resistive heater, a printed circuit board heater, a flexible circuit heater, or a combination thereof.

92. The system of any one of claims 37-91, further comprising a supplementary cooling device, wherein the supplementary cooling device is a heatsink, a fan, a thermoelectric device, a Peltier cooler, or a combination thereof.

93. The system of any one of claims 37-92, wherein said system is configured to conduct a PCR reaction at or in close proximity of the surface of the reaction zone.

94. The system of any one of claims 37-93, further comprising a reaction zone, wherein the reaction zone is configured into at least two separate regions comprising one region configured to perform liquid-phase PCR and another region configured to detect an amplification product captured on a capture surface.

95. The system of claim 94, wherein the capture surface is modified with a linker layer.

96. The system of claim 95, wherein the linker layer is configured to bind to a double or single stranded DNA or RNA strand via the 3′ or the 5′ end.

97. The system of any one of claims 95-96, wherein the linker layer comprises silane or small chemical molecules with one or more reactive functional chemical terminal groups.

98. The system of any one of claims 95-97, wherein the linker layer comprises single-stranded DNA or RNA bound to the silane or small chemical molecules.

99. The system of any one of claims 95-98, wherein the linker layer comprises one or more polymers.

100. The system of claim 99, wherein the polymer is a form of dextran, carboxymethyl dextran, chitosan, polyaniline, PEG, PLL-PEG, PLL-g-PEG, PLA-PEG-PLL, or a combination thereof.

101. The system of any one of claims 95-100, wherein the linker layer comprises single-stranded DNA or RNA bound to the polymer.

102. The system of any one of claims 95-101, wherein the linker layer comprises microparticles and/or nanoparticles.

103. The system of claim 102, wherein the microparticles and/or nanoparticles are composed of metal, semiconductor, compound semiconductor, polymer, plastic, oxide, glass, or a combination thereof.

104. The system of any one of claims 102-103, wherein the microparticles and/or nanoparticles are configured to bind to silane and/or small chemical molecules of the linker layer.

105. The system of any one of claims 102-104, wherein the microparticles and/or nanoparticles comprises a surface, and wherein the surface of the microparticles and/or nanoparticles is at least partially modified with silane and/or small chemical molecules having reactive functional chemical terminal groups to bind with the silane and/or chemical molecules of the linker layer.

106. The system of any one of claims 102-105, wherein the microparticles and/or nanoparticles are configured to bind to the DNA or RNA strands of the linker layer.

107. The system of any one of claims 102-106, wherein one or more primer strands required for the PCR reaction to amplify a particular DNA/RNA target is chemically or physically bound to the linker layer of any one of claims 94-106.

108. The system of claim 107, wherein the one or more primer strands comprise forward primer strands or reverse primer strands for a particular DNA/RNA target.

109. The system of any one of claims 107-108, wherein the one or more primer strands comprise one set of primer strands for a particular DNA/RNA target, and wherein the one set of primer strands, either the forward or reverse strand, is bound to the linker layer.

110. The system of claim 109, wherein a complementary set of primer strands to the one set of primer strands, is present in the liquid sample.

111. The system of any one of claims 108-110, wherein both the forward and reverse primer strands for a particular DNA/RNA target are bound to the linker layer.

112. The system of any one of claims 37-111, wherein the reader device is a desktop or portable device configured to receive said test cartridge, to perform liquid-phase PCR, and to detect an amplification product.

113. The system of claim 112, wherein the reader device is further configured to provide energy, from the energy source, for heating and/or cooling one or more reaction zones on the test cartridge.

114. The system of any one of claims 112-113, wherein the reader device is further configured to monitor the temperature, with the thermal sensor, of one or more reaction zones on the test cartridge.

115. The system of any one of claims 112-114, wherein the reader device is further configured to adjust the energy output of the energy source to one or more reaction zones on the test cartridge, based on the readings of the thermal sensor, to maintain a selected temperature.

116. The system of any one of claims 112-115, wherein the reader device is further configured to activate and/or deactivate a supplementary heating and cooling devices to adjust the temperature of one or more reaction zones on the test cartridge.

117. The system of any one of claims 112-116, wherein the reader device is further configured to excite one or more reaction zones on the test cartridge with light of one or more excitation wavelengths using the excitation source.

118. The system of any one of claims 112-117, wherein the reader device is further configured to detect and measure the light emitted from one or more reaction zones on the test cartridge, with a light sensor, and convert the readings into one or more output signals.

119. The system of any one of claims 112-118, wherein the reader device is further configured to display the one or more output signals on the reader.

120. The system of any one of claims 112-119, wherein the reader device is further configured to display or transmit the one or more output signals on another device via a wired or wireless connection.

121. A method for amplifying and detecting nucleic acids on a point-of-care system, the method comprising:
providing the point-of-care system of any one of claims 37-120;
receiving a liquid sample containing PCR components at the test cartridge;
modulating an energy output of the energy source to alternatively heat and cool the liquid sample to amplify nucleic acids;
amplifying sample nucleic acids in the liquid sample; and
measuring or detecting an amplification product using the reader device.

122. The method of claim 121, further comprising dispensing a liquid sample containing PCR components, including at least one target-specific primer, and target DNA or RNA into a reaction zone.

123. The method of any one of claims 121-122, further comprising measuring a baseline temperature of the liquid sample with a thermal sensor.

124. The method of any one of claims 121-123, further comprising monitoring the temperature of the heat generation layer and/or liquid sample with the thermal sensor.

125. The method of any one of claims 121-124, further comprising adjusting the energy output of the energy source, based on measurements from the thermal sensor, to reach and maintain the temperature of the liquid sample at the optimal denaturing temperature of the target DNA for the duration of an initial denaturing period.

126. The method of claim 125, further comprising allowing the initial denaturing step to continue for a preset duration such that the target DNA in the sample is fully denatured.

127. The method of any one of claims 121-126, further comprising reducing the energy output of the energy source until the temperature of the liquid sample reaches an optimal primer annealing temperature, as measured by the thermal sensor.

128. The method of claim 127, further comprising adjusting the energy output from the energy source, based on measurements from the thermal sensor, to reach and maintain the temperature of the liquid sample at the optimal primer annealing temperature.

129. The method of any one of claims 127-128, further comprising allowing primer annealing to continue for a preset duration such that both the forward and reverse primers fully hybridize to the denatured target DNA strands.

130. The method of any one of claims 127-129, further comprising increasing the energy output from the energy source until the temperature of the liquid sample reaches the optimal primer extension temperature, as measured by the thermal sensor.

131. The method of claim 130, further comprising adjusting the energy output from the energy source, based on measurements from the thermal sensor, to reach and maintain the temperature of the liquid sample at the optimal primer extension temperature.

132. The method of any one of claims 130-131, further comprising allowing primer extension to continue for a preset duration such that the target DNA strand is extended with free nucleotides or free nucleotides labeled with one or more molecules.

133. The method of any one of claims 121-132, further comprising repeating primer annealing and primer extensions for a desired number of cycles by adjusting energy output of the energy source and monitoring the temperature of the liquid sample with the thermal sensor.

134. The method of any one of claims 121-133, further comprising deactivating the energy source to let the temperature of the liquid sample to return to a preset lower temperature while monitoring the temperature of the liquid sample with the thermal sensor.

135. The method of any one of claims 121-133, further comprising measuring fluorescence output from the liquid sample by exciting the sample with an excitation source and measuring the resulting emission with a light sensor having the appropriate filter lens or lenses.

136. The method of any one of claims 121-135, further comprising performing a final denaturing step prior to measuring fluorescence output.

137. The method of claim 136, wherein prior to measuring the fluorescence output, the method further comprises:
dispensing primers tagged with a fluorescent and a quencher molecule into the liquid sample;
increasing the temperature of the liquid sample to the denaturing temperature of the target DNA for a preset duration;
decreasing the temperature of the liquid sample to a primer annealing temperature of the tagged primers for a preset duration to allow the tagged primers to bind with the amplified target DNA in the liquid sample; and
decreasing the temperature of the liquid sample to allow for optimal fluorescence detection.

138. The method of any one of claims 121-137, wherein the primers contain a fluorescent dye molecule attached to the 5′ or 3′ prime terminal end but not both.

139. The method of any one of claims 121-138, wherein the primers contain a quencher molecule attached to 3′ or 5′ prime terminal end but not both.

140. The method of any one of claims 121-139, wherein the primers form a hairpin loop structure when not bound to target amplified DNA, such that fluorescence from the fluorescent molecule is quenched by the quencher molecule.

141. The method of any one of claims 121-140, wherein the primers comprise a nucleotide sequence which is complementary to at least a portion of one of the denatured strands of amplified target DNA.

142. The method of any one of claims 121-141, wherein the primers elongate and hybridize to the denatured target amplified DNA during the primer anneal step, such that fluorescence from the fluorescent molecule is not quenched by the quencher molecule.

143. A method for performing isothermal PCR reactions, using the system of claim 37-120, the method comprising:
dispensing a liquid sample containing components for isothermal PCR, comprising recombinase polymerase reaction, loop-mediated isothermal PCR, strand displacement amplification, helicase-dependent amplification, or nicking enzyme amplification, into the sample confinement layer and/or reaction zone(s);
performing isothermal amplification for a preset duration;
capturing amplified target DNA of the PCR reaction product; and
detecting the captured amplified target DNA of the PCR reaction product.

144. The method of claim 143, further comprising measuring a baseline temperature of the liquid sample with the thermal sensor.

145. The method of any one of claims 143-144, further comprising monitoring the temperature of the heat generation layer and/or liquid sample with the thermal sensor.

146. The method of any one of claims 143-145, further comprising adjusting the energy output of the energy source, based on measurements from the thermal sensor, to reach and maintain the temperature of the liquid sample at the optimal denaturing temperature of the target DNA for the duration of the initial denaturing period.

147. The method of any one of claims 143-146, further comprising allowing the initial denaturing step to continue for a preset duration such that the target double-stranded DNA in the sample is fully denatured.

148. The method of any one of claims 143-147, further comprising reducing the energy output of the energy source until the temperature of the liquid sample reaches the optimal temperature for primal annealing and isothermal amplification, as measured by the thermal sensor.

149. The method of any one of claims 143-148, further comprising adjusting the energy output from the energy source, based on measurements from the thermal sensor, to reach and maintain the temperature of the liquid sample at the optimal temperature for isothermal amplification for the duration of the amplification step.

150. The method of any one of claims 143-149, further comprising deactivating the energy source to let the temperature of the liquid sample to return to a preset lower temperature while monitoring the temperature of the liquid sample with the thermal sensor.
